(19) United States
(12) Patent Application Publication
(10) Pub. No.: US 2024/0270731 A1
(43) Pub. Date: Aug. 15, 2024

(54) DEUTERATED COMPOUNDS

(71) Applicant: ALEBUND PHARMACEUTICALS (HONG KONG) LIMITED, Hong Kong (CN)

(72) Inventors: Chutian SHU, Shanghai (CN); Jin TIAN, Shanghai (CN)

(21) Appl. No.: 18/293,337

(22) PCT Filed: Aug. 9, 2022
(86) PCT No.: PCT/CN2022/111089

§ 371 Date: Jan. 29, 2024

(30) Foreign Application Priority Data
Aug. 13, 2021 (WO) PCT/CN2021/112521
Jul. 20, 2022 (WO) PCT/CN2022/106648

(51) Int. Cl. C07D 413/12; A61K 31/422; A61P 3/06; A61P 3/10; A61P 13/12

(52) U.S. Cl. CPC C07D 413/12; A61K 31/422; A61P 3/06; A61P 3/10; A61P 13/12

(57) ABSTRACT

Disclosed herein are compounds according to formula (I), the pharmaceutical composition comprising the same, and uses thereof.

### TECHNICAL FIELD

The present disclosure generally relates to the field of pharmaceuticals and to methods of treating disorders. More particularly, provided herein are novel compounds which are dual agonists of PPARα and PPARγ and are useful in the treatment and/or prophylaxis of diseases such as diabetes, dyslipidemia, or diabetic nephropathy.

### BACKGROUND

Diabetes is a disease in which a patient's ability to control glucose levels in blood is impaired, because he has partially lost the ability to respond properly to the action of insulin. In type II diabetes (T2D), often referred to as non-insulin dependent diabetes mellitus (NIDDM), which afflicts 80-90% of all diabetic patients in developed countries, the Isles of Langerhans in the pancreas still produce insulin. However, the target organs, mainly muscle, liver and adipose tissue, exhibit a profound resistance to insulin stimulation, and the body compensates by producing unphysiologically high levels of insulin. In later stage of disease, however, insulin secretion decreases due to exhaustion of the pancreas. Moreover, T2D is a metabolic-cardiovascular disease syndrome. The comorbidities associated with T2D include insulin resistance, dyslipidemia, hypertension, endothelial dysfunction and inflammatory atherosclerosis.

Peroxisome Proliferator Activated Receptors (PPAR's) are members of the nuclear hormone receptor super family, which are ligand-activated transcription factors regulating gene expression. Various subtypes thereof have been identified and cloned. These include PPARα, PPARβ (also known as PPARδ), and PPARγ. At least two major isoforms of PPARγ exist. While PPARγ1 is ubiquitously expressed in most tissues, the longer isoform PPARγ2 is almost exclusively found in adipocytes. In contrast, PPARα is predominantly expressed in the liver, kidney and heart. PPAR's modulate a variety of body responses including glucose- and lipid-homeostasis, cell differentiation, inflammatory responses and cardiovascular events. It is of great significance for clinical drug development to combine the lipid metabolism regulation activity of PPARα and the insulin sensitivity regulation activity of PPARγ to develop specific PPAR-α/γ dual agonists to control blood sugar and improve cardiovascular symptoms.

As a PPAR-α/γ dual agonist, aleglitazar has a relatively balanced PPAR-α/γ activity. Aleglitazar can effectively improve fasting and postprandial blood sugar levels, insulin sensitivity and blood lipid parameters. However, the results of phase 3 clinical trials of aleglitazar showed that although it can effectively reduce blood sugar and blood lipid levels, it also brings a certain degree of heart failure risk, so it does not produce corresponding cardiovascular benefits. At the same time, adverse reactions such as fracture risk have also been found in clinical trial, and it's known that these adverse reactions are caused by PPARγ activation (Lincoff A M, et al. JAMA. 2014; 311(15):1515-1525). Therefore, it is necessary to develop suitable selective PPAR-α/γ dual agonists to improve safety and benefit patients.

The present disclosure develops a series of novel dual agonist of PPARα and PPARγ by deuterating aleglitazar. These compounds have PPAR-α/γ selectivity different from aleglitazar. Some of these compounds may have a stronger PPARα activity, and thus can display better PPARα activity in in vitro transcription and in vivo lipid reduction, while maintaining a certain degree of PPARγ activity. Therefore, they can still have good therapeutic effects in regulating the levels of blood lipid and blood sugar, and meanwhile can reduce the side effects caused by PPARγ activity, such as weight gain and the risk of heart failure. Compared with aleglitazar, these compounds may have a more reasonable risk-benefit ratio for patients suffering from metabolic syndrome, and have good clinical application prospects.

### BRIEF SUMMARY OF THE INVENTION

The present disclosure provides deuterated aleglitazar, the pharmaceutical composition comprising the same, and uses thereof.

In one aspect, the present disclosure provides a compound of formula (I), or its pharmaceutical acceptable salt,

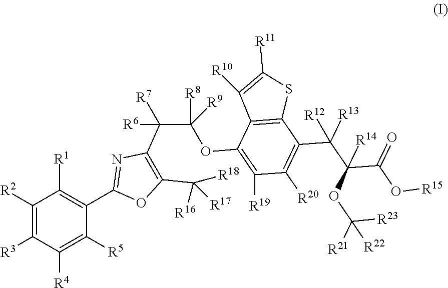

wherein, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ independently from each other are H or D, and at least one, two, three, four, five, six, seven, eight, nine, ten, eleven, twelve, thirteen, fourteen, fifteen, sixteen, seventeen, eighteen, nineteen, twenty, twenty one, twenty two or twenty three of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ is D.

In certain embodiments, no more than one, two, three, four, five, six, seven, eight, nine, ten, eleven, twelve, thirteen, fourteen, fifteen, sixteen, seventeen, eighteen, nineteen, twenty, twenty one, twenty two or twenty three of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ is D.

In certain embodiments, at least one, two, three or four of R⁶, R⁷, R⁸ and R⁹ is D.

In certain embodiments, no more than one, two, three or four of R⁶, R⁷, R⁸ and R⁹ is D.

In certain embodiments, one or two of R⁶ or R⁷ is D.

In certain embodiments, one or two of R⁸ or R⁹ is D.

In certain embodiments, all of R¹, R², R³, R⁴, R⁵, R¹⁰, R¹¹, R¹², R¹³, R¹⁴, R¹⁵, R¹⁶, R¹⁷, R¹⁸, R¹⁹, R²⁰, R²¹, R²² and R²³ are H.

In certain embodiments, all of R¹, R², R³, R⁴, R⁵, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ are H.

In certain embodiments, all of R¹, R², R³, R⁴, R⁵, R⁶, R⁷, R¹⁰, R¹¹, R¹², R¹³, R¹⁴, R¹⁵, R¹⁶, R¹⁷, R¹⁸, R¹⁹, R²⁰, R²¹, R²² and R²³ are H.

In certain embodiments, at least one, two, three, four or five of R¹, R², R³, R⁴ and R⁵ is D.

In certain embodiments, no more than one, two, three, four or five of R¹, R², R³, R⁴ and R⁵ is D.

In certain embodiments, all of R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ are H.

In certain embodiments, all of R¹, R², R³, R⁴ and R⁵ are D, and all of R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ are H.

In certain embodiments, at least one, two, three, four, five, six, seven, eight or nine of R¹, R², R³, R⁴, R⁵, R⁶, R⁷, R⁸ and R⁹ is D.

In certain embodiments, no more than one, two, three, four, five, six, seven, eight or nine of R¹, R², R³, R⁴, R⁵, R⁶, R⁷, R⁸ and R⁹ is D.

In certain embodiments, at least one, two, three, four or five of R¹, R², R³, R⁴ and R⁵ is D and at least one, two, three, or four of R⁶, R⁷, R⁸ and R⁹ is D.

In certain embodiments, at least one, two, three, four or five of R¹, R², R³, R⁴ and R⁵ is D and at least one or two of R⁸ and R⁹ is D.

In certain embodiments, at least one or two of R¹ and R⁵ is D and at least one or two of R⁸ and R⁹ is D.

In certain embodiments, all of R¹⁰, R¹¹, R¹², R¹³, R¹⁴, R¹⁵, R¹⁶, R¹⁷, R¹⁸, R¹⁹, R²⁰, R²¹, R²² and R²³ are H.

In certain embodiments, at least one, two or three of R¹², R¹³ and R¹⁴ is D.

In certain embodiments, no more than one, two or three of R¹², R¹³ and R¹⁴ is D.

In certain embodiments, at least one or two of R¹² and R¹³ is D and R¹⁴ is D.

In certain embodiments, all of R¹, R², R³, R⁴, R⁵, R⁶, R⁷, R⁸, R⁹, R¹⁰, R¹¹, R¹⁵, R¹⁶, R¹⁷, R¹⁸, R¹⁹, R²⁰, R²¹, R²² and R²³ are H.

In certain embodiments, at least one, two or three of R¹⁶, R¹⁷ and R¹⁸ is D.

In certain embodiments, no more than one, two or three of R¹⁶, R¹⁷ and R¹⁸ is D.

In certain embodiments, all of R¹, R², R³, R⁴, R⁵, R⁶, R⁷, R⁸, R⁹, R¹⁰, R¹¹, R¹², R¹³, R¹⁴, R¹⁵, R¹⁹, R²⁰, R²¹, R²² and R²³ are H.

In certain embodiments, at least one, two, three, four, five, six, seven, eight, nine, ten, eleven or twelve of R¹, R², R³, R⁴, R⁵, R⁶, R⁷, R⁸, R⁹, R¹⁶, R¹⁷ and R¹⁸ is D.

In certain embodiments, no more than one, two, three, four, five, six, seven, eight, nine, ten, eleven or twelve of R¹, R², R³, R⁴, R⁵, R⁶, R⁷, R⁸, R⁹, R¹⁶, R¹⁷ and R¹⁸ is D.

In certain embodiments, at least one or two of R⁸ and R⁹ is D and at least one, two or three of R¹⁶, R¹⁷ and R¹⁸ is D.

In certain embodiments, at least one, two, three, four or five of R¹, R², R³, R⁴, and R⁵ is D and at least one, two or three of R¹⁶, R¹⁷ and R¹⁸ is D.

In certain embodiments, all of, R¹⁰, R¹¹, R¹², R¹³, R¹⁴, R¹⁵, R¹⁹, R²⁰, R²¹, R²² and R²³ are H.

In another aspect, the present disclosure provides a compound of formula (Ia), or its pharmaceutical acceptable salt,

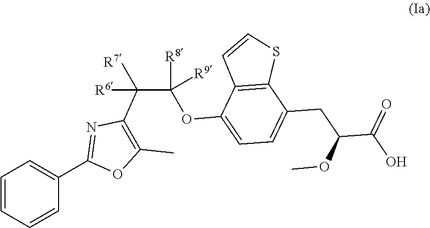

wherein, R^{6′}, R^{7′}, R^{8′} and R^{9′} independently from each other are H or D, wherein at least one, two, three or four of R^{6′}, R^{7′}, R^{8′} and R^{9′} is D.

In certain embodiments, no more than one, two, three or four of R^{6′}, R^{7′}, R^{8′} and R^{9′} is D.

In certain embodiments, at least one or two of R^{8′} and R^{9′} is D.

In certain embodiments, at least one or two of R^{6′} and R^{7′} is D.

In certain embodiments, both of R^{6′} and R^{7′} are H when at least one or two of R^{8′} and R^{9′} is D.

In certain embodiments, both of R^{6′} and R^{7′} are H when both of R^{8′} and R^{9′} is D.

In certain embodiments, both of R^{8′} and R^{9′} are H when at least one or two R^{6′} and R^{7′} of is D.

In another aspect, the present disclosure provides a compound selected from

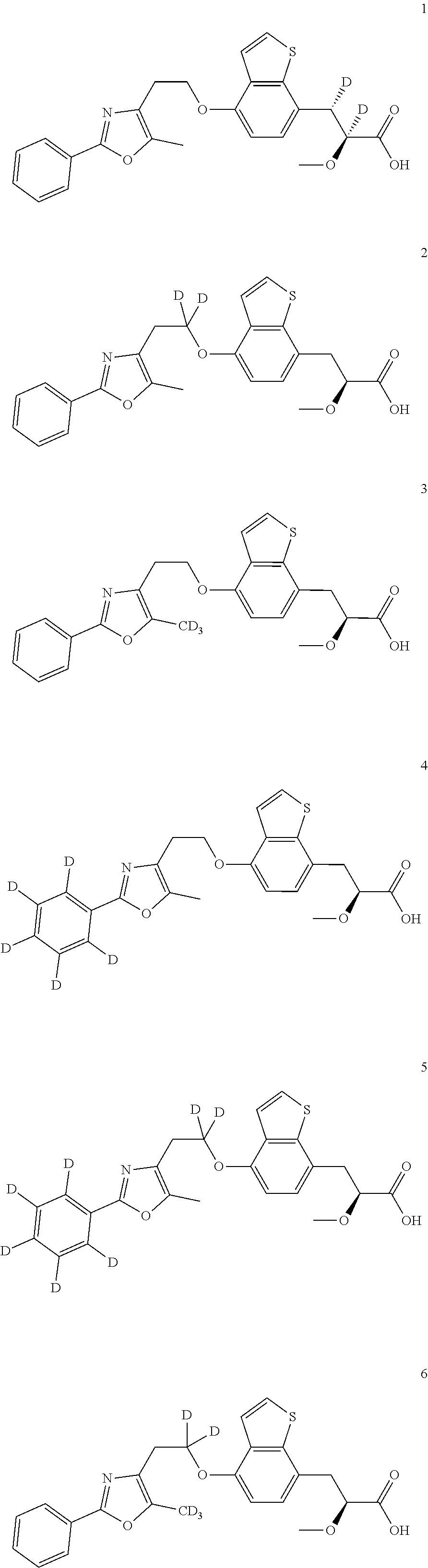

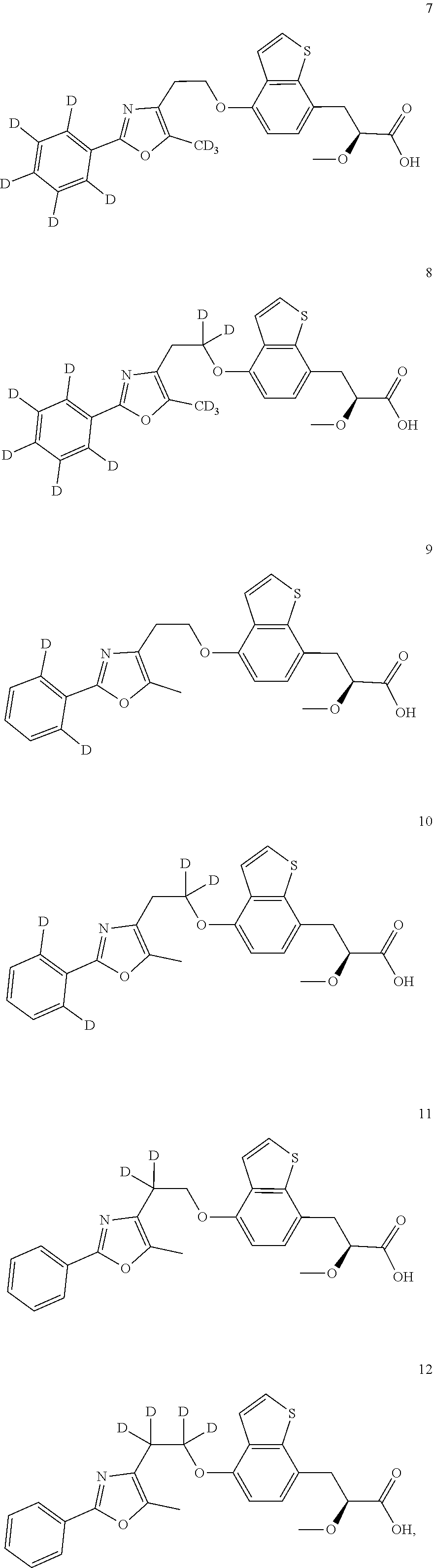

or its pharmaceutical acceptable salt.

In certain embodiments, deuterium enrichment is no less than 50%, 60%, 70%, 80%, 90%, 95%, 96%, 97%, 98% or 99%.

In certain embodiments, deuterium enrichment is no more than 99.9%, 99%, 98%, 97%, 96%, 95%, or 90%.

In another aspect, the present disclosure provides a pharmaceutical composition comprising a compound or its pharmaceutical acceptable salt provided herein and a pharmaceutically acceptable carrier and/or adjuvant.

In another aspect, the present disclosure provides a dual agonist of PPARα and PPARγ for use in a method for the treatment and/or prophylaxis of diseases which are modulated by PPARα and/or PPARγ agonists, wherein the dual agonist of PPARα and PPARγ is deuterated.

In another aspect, the present disclosure provides a method for the treatment and/or prophylaxis of diseases which are modulated by PPARα and/or PPARγ agonists in a subject, comprising administering a dual agonist of PPARα and PPARγ to the subject, wherein the dual agonist of PPARα and PPARγ is deuterated.

In another aspect, the present disclosure provides the use of a dual agonist of PPARα and PPARγ in the manufacture of a medicament for the treatment and/or prophylaxis of diseases which are modulated by PPARα and/or PPARγ agonists, wherein the dual agonist of PPARα and PPARγ is deuterated.

In certain embodiments, the disease is diabetes, non-insulin dependent diabetes mellitus, elevated blood pressure, dyslipidemia, atherosclerotic diseases, metabolic syndrome, or diabetic nephropathy.

In certain embodiments, the disease is renal injury.

In certain embodiments, the renal injury is caused by ureteral obstruction.

In certain embodiments, the renal injury is caused by unilateral ureteral obstruction.

In certain embodiments, the dual agonist of PPARα and PPARγ is deuterated Aleglitazar or its pharmaceutical acceptable salt.

In certain embodiments, the dual agonist of PPARα and PPARγ is the compound or its pharmaceutical acceptable salt provided herein.

In another aspect, the present disclosure provides a method for modulating the specific agonistic activity of PPARα or PPARγ of a dual agonist of PPARα and PPARγ, comprising deuterating the agonist.

In another aspect, the present disclosure provides a method for improving the specific agonistic activity of PPARα or PPARγ of a dual agonist of PPARα and PPARγ, comprising deuterating the agonist.

In certain embodiments, the specific agonistic activity of PPARα of the agonist is improved.

In certain embodiments, the specific agonistic activity of PPARγ of the agonist is improved.

In certain embodiments, at least one, two, three, four, five, six, seven, eight, nine, ten of H of the agonist is deuterated.

In certain embodiments, no more than one, two, three, four, five, six, seven, eight, nine, ten of H of the agonist is deuterated.

In certain embodiments, the dual agonist of PPARα and PPARγ is Aleglitazar or its pharmaceutical acceptable salt.

### BRIEF DESCRIPTION OF FIGURES

The following drawings form part of the present specification and are included to further demonstrate certain aspects of the present invention. The invention may be better understood by reference to one or more of these drawings in combination with the detailed description of specific embodiments presented herein.

FIG. 1A shows changes of serum TG in each group. Data were presented as Mean±SD; One way ANOVA by Prism GraphPad; n=6. **** P<0.0001, *** P<0.001, ** P<0.001, *P<0.05 vs Vehicle. FIG. 1B shows changes of serum NEFA in each group. Data were presented as Mean±SD; One way ANOVA by Prism GraphPad; n-6. **** P<0.0001 vs Vehicle.

FIG. 2A shows changes in body weight gain of each group of animals compared with Day 0. FIG. 2B shows changes in the difference in weight gain between each group of animals and the vehicle group. (*: The weight gain of the treatment group minus the average weight gain of the vehicle group.)

FIG. 3 shows the effect of the compound on the changes of db/db animal's body weight. Data were presented as Mean±SEM; Two way ANOVA followed by Dunnett test by Prism GraphPad; n=6-9.

FIG. 4A shows the effect of the compounds on serum TG levels in db/db animals on Day 6. Data were presented as Mean±SEM; One way ANOVA followed by Dunnett test by Prism GraphPad; n=6-9. ** P<0.01, *** P<0.001, **** P<0.0001 vs Model. FIG. 4B shows the effect of the compounds on serum TG levels in db/db animals on Day 12. Data were presented as Mean±SEM; One way ANOVA followed by Dunnett test by Prism GraphPad; n=6-9. **** P<0.0001 vs Model.

FIG. 5 shows the effect of compounds on random blood glucose of db/db animals. Data were presented as Mean±SEM, n=6-9.

FIG. 6A shows the effect of compounds on oral glucose tolerance of db/db animals. Data were presented as Mean±SEM, n=6-9. FIG. 6B shows the effect of compounds on oral glucose tolerance of db/db animals. Data were presented as Mean±SEM; One way ANOVA followed by Dunnett test by Prism GraphPad; n=6-9. ** P<0.01, *** P<0.001, **** P<0.0001 vs model group.

FIG. 7 shows the effect of Compound 2 on urinary albumin excretion.

FIG. 8A-8D shows the effect of Compound 2 on Glomerular and Tubular damage.

FIG. 9 shows the effect of Compound 2 on improvement of renal injury in rat model of unilateral ureteral obstruction.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

As used herein, the singular forms “a”, “an” and “the” may refer to plural articles unless specifically stated otherwise.

The term “about” or “approximately” as used herein should be considered as disclosing the range defined by the absolute values of the two endpoints. The term “about” or “approximately” also means an acceptable error for a particular value, which depends in part on how the value is measured or determined. In certain embodiments, “about” can mean 1 or more standard deviations. For example, the expression “from about 2 to about 4” also discloses the range “from 2 to 4”. When used to modify a single number, the term “about” may refer to plus or minus 10% of the indicated number and includes the indicated number. For example, “about 10%” may indicate a range of 9% to 11%, and “about 1” may indicate a range of 0.9-1.1.

### Therapeutic Compounds

The present invention provides a compound of formula (I), or its pharmaceutical acceptable salt,

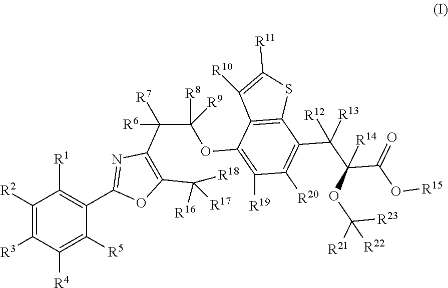

wherein, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ independently from each other are H or D, and at least one, two, three, four, five, six, seven, eight, nine, ten, eleven, twelve, thirteen, fourteen, fifteen, sixteen, seventeen, eighteen, nineteen, twenty, twenty one, twenty two or twenty three of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ is D.

In certain embodiments, a range of between one and twenty three, between one and twenty two, between one and twenty one, between one and twenty, between one and nineteen, between one and eighteen, between one and seventeen, between one and sixteen, between one and fifteen, between one and fourteen, between one and thirteen, between one and twelve, between one and eleven, between one and ten, between one and nine, between one and eight, between one and seven, between one and six, between one and five, between one and four, between one and three, or between one and two of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ are D.

In certain embodiments, a range of between one and six of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ are D.

In certain embodiments, a range of between one and four of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ are D.

In certain embodiments, one or two of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ are D.

In certain embodiments, at least one, two, three or four of R⁶, R⁷, R⁸ and R⁹ is D.

In certain embodiments, a range of between one and four, between one and three, or between one and two of R⁶, R⁷, R⁸ and R⁹ are D.

In certain embodiments, four of R⁶, R⁷, R⁸ and R⁹ are D.

In certain embodiments, two of R⁶, R⁷, R⁸ and R⁹ are D.

In certain embodiments, one or two of R⁶ or R⁷ is D.

In certain embodiments, one of R⁶ or R⁷ is D.

In certain embodiments, R⁶ or R⁷ are both D.

In certain embodiments, one or two of R⁸ or R⁹ is D.

In certain embodiments, one of R⁸ or R⁹ is D.

In certain embodiments, R⁸ or R⁹ are both D.

In certain embodiments, all of R¹, R², R³, R⁴, R⁵, R¹⁰, R¹¹, R¹², R¹³, R¹⁴, R¹⁵, R¹⁶, R¹⁷, R¹⁸, R¹⁹, R²⁰, R²¹, R²² and R²³ are H.

In certain embodiments, all of R¹, R², R³, R⁴, R⁵, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ are H.

In certain embodiments, all of R¹, R², R³, R⁴, R⁵, R⁶, R⁷, R¹⁰, R¹¹, R¹², R¹³, R¹⁴, R¹⁵, R¹⁶, R¹⁷, R¹⁸, R¹⁹, R²⁰, R²¹, R²² and R²³ are H.

In certain embodiments, at least one, two, three, four or five of R¹, R², R³, R⁴ and R⁵ is D.

In certain embodiments, a range of between one and five, between one and four, between one and three, or between one and two of R¹, R², R³, R⁴ and R⁵ are D.

In certain embodiments, R¹, R², R³, R⁴ and R⁵ are all D.

In certain embodiments, all of R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ are H.

In certain embodiments, all of R¹, R², R³, R⁴ and R⁵ are D, and all of R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ are H.

In certain embodiments, at least one, two, three, four, five, six, seven, eight or nine of R¹, R², R³, R⁴, R⁵, R⁶, R⁷, R⁸ and R⁹ is D.

In certain embodiments, a range of between one and nine, between one and eight, between one and seven, between one and six, between one and five, between one and four, between one and three, or between one and two of R¹, R², R³, R⁴, R⁵, R⁶, R⁷, R⁸ and R⁹ is D.

In certain embodiments, at least one, two, three, four or five of R¹, R², R³, R⁴ and R⁵ is D and at least one, two, three, or four of R⁶, R⁷, R⁸ and R⁹ is D.

In certain embodiments, at least one, two, three, four or five of R¹, R², R³, R⁴ and R⁵ is D and at least one or two of R⁸ and R⁹ is D.

In certain embodiments, at least one or two of R¹ and R⁵ is D and at least one or two of R⁸ and R⁹ is D.

In certain embodiments, R¹, R², R³, R⁴, R⁵, R⁶, R⁷, R⁸ and R⁹ are all D.

In certain embodiments, all of R¹⁰, R¹¹, R¹², R¹³, R¹⁴, R¹⁵, R¹⁶, R¹⁷, R¹⁸, R¹⁹, R²⁰, R²¹, R²² and R²³ are H.

In certain embodiments, at least one, two or three of R¹², R¹³ and R¹⁴ is D.

In certain embodiments, a range of between one and three, or between one and two of R¹², R¹³ and R¹⁴ are D.

In certain embodiments, one of R¹², R¹³ and R¹⁴ is D.

In certain embodiments, at least one or two of R¹² and R¹³ is D and R¹⁴ is D.

In certain embodiments, R¹² and R¹⁴ are both D. In certain embodiments, R¹³ and R¹⁴ are both D. In certain embodiments, R¹², R¹³ and R¹⁴ are all D.

In certain embodiments, all of R¹, R², R³, R⁴, R⁵, R⁶, R⁷, R⁸, R⁹, R¹⁰, R¹¹, R¹⁵, R¹⁶, R¹⁷, R¹⁸, R¹⁹, R²⁰, R²¹, R²² and R²³ are H.

In certain embodiments, at least one, two or three of R¹⁶, R¹⁷ and R¹⁸ is D.

In certain embodiments, a range of between one and three, or between one and two of R¹⁶, R¹⁷ and R¹⁸ are D.

In certain embodiments, one of R¹⁶, R¹⁷ and R¹⁸ is D.

In certain embodiments, all of R¹, R², R³, R⁴, R⁵, R⁶, R⁷, R⁸, R⁹, R¹⁰, R¹¹, R¹², R¹³, R¹⁴, R¹⁵, R¹⁹, R²⁰, R²¹, R²² and R²³ are H.

In certain embodiments, at least one, two, three, four, five, six, seven, eight, nine, ten, eleven or twelve of R¹, R², R³, R⁴, R⁵, R⁶, R⁷, R⁸, R⁹, R¹⁶, R¹⁷ and R¹⁸ is D.

In certain embodiments, a range of between one and twelve, between one and eleven, between one and ten, between one and nine, between one and eight, between one and seven, between one and six, between one and five, between one and four, between one and three, or between one and two of R¹, R², R³, R⁴, R⁵, R⁶, R⁷, R⁸, R⁹, R¹⁶, R¹⁷ and R¹⁸ are D.

In certain embodiments, at least one or two of R⁸ and R⁹ is D and at least one, two or three of R¹⁶, R¹⁷ and R¹⁸ is D.

In certain embodiments, at least one, two, three, four or five of R¹, R², R³, R⁴, and R⁵ is D and at least one, two or three of R¹⁶, R¹⁷ and R¹⁸ is D.

In certain embodiments, R¹, R², R³, R⁴, R⁵, R⁶, R⁷, R⁸, R⁹, R¹⁶, R¹⁷ and R¹⁸ are all D.

In certain embodiments, all of R¹⁰, R¹¹, R¹², R¹³, R¹⁴, R¹⁵, R¹⁹, R²⁰, R²¹, R²² and R²³ are H.

In another aspect, the present invention provides a compound of formula (Ia), or its pharmaceutical acceptable salt,

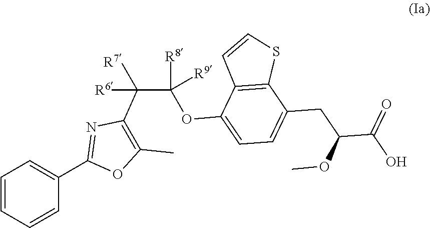

wherein, R^{6′}, R^{7′}, R^{8′} and R^{9′} independently from each other are H or D, wherein at least one, two, three or four of R^{6′}, R^{7′}, R^{8′} and R^{9′} is D.

In certain embodiments, no more than one, two, three or four of R^{6′}, R^{7′}, R^{8′} and R^{9′} is D.

In certain embodiments, a range of between one and four, between one and three, or between one and two of R^{6′}, R^{7′}, R^{8′} and R^{9′} are D.

In certain embodiments, at least one or two of R^{8′} and R^{9′} is D.

In certain embodiments, R^{8′} and R^{9′} are both D.

In certain embodiments, at least one or two of R^{6′} and R^{7′} is D.

In certain embodiments, both of R^{6′} and R^{7′} are H when at least one or two of R^{8′} and R^{7′} is D.

In certain embodiments, both of R^{6′} and R^{7′} are H when both of R^{8′} and R^{9′} is D.

In certain embodiments, both of R^{6′} and R^{7′} are H and both of R^{8′} and R^{9′} are D.

In certain embodiments, both of R^{8′} and R^{9′} are H when at least one or two R^{6′} and R⁷ of is D.

In certain embodiments, both of R^{8′} and R^{9′} are H and both R^{6′} and R^{7′} of are D.

In another aspect, the present invention provides a compound selected from

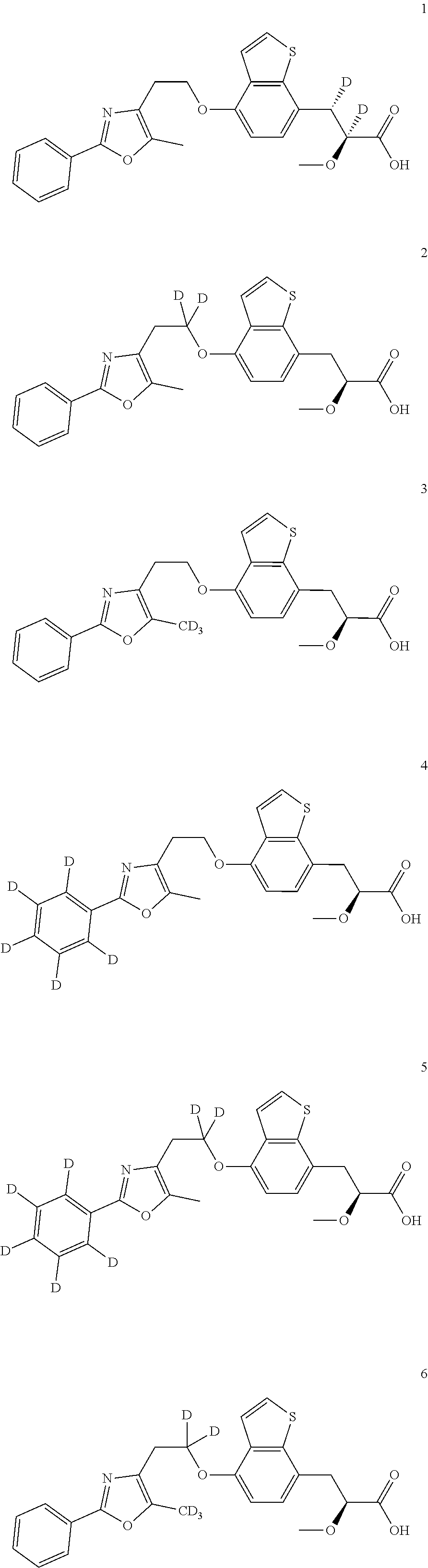

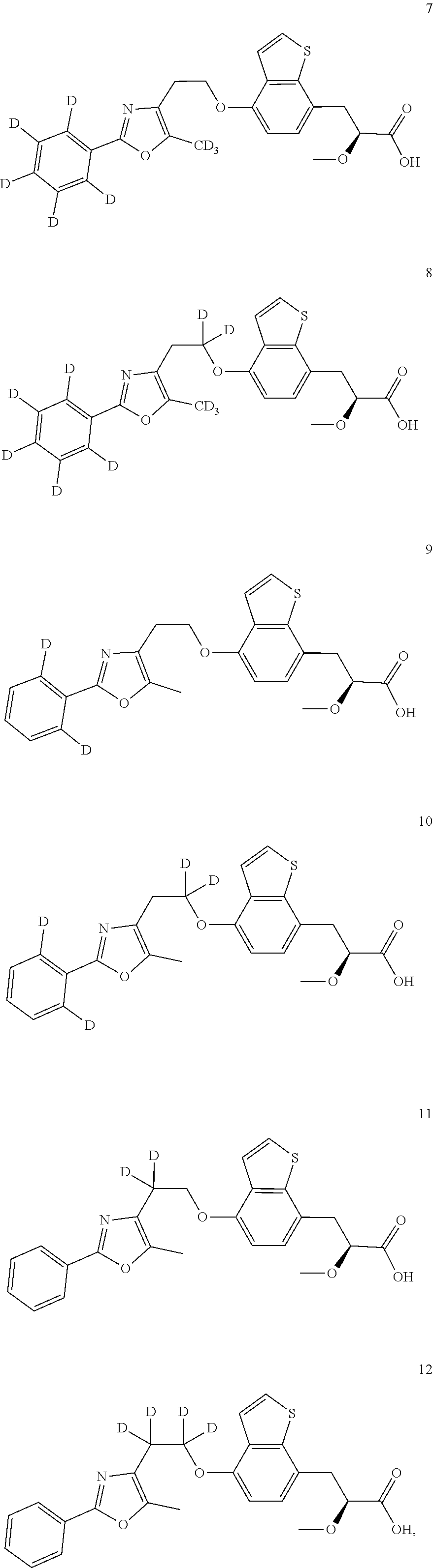

or its pharmaceutical acceptable salt.

In certain embodiments, the compound is

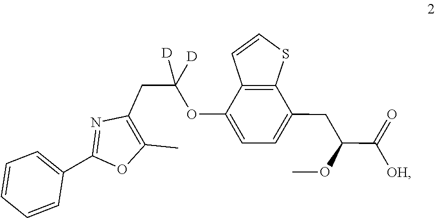 [SMILES: [2H]C([2H])(Cc1nc(-c2ccccc2)oc1C)Oc1ccc(C[C@H](OC)C(=O)O)c2sccc12]

or its pharmaceutical acceptable salt.

The term “compound”, when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound.

The term “is/are deuterium/D”, when used to describe a given position in a molecule or a drawing of a molecular structure, means that the specified position is deuterium or that the specified position is enriched with deuterium above the naturally occurring distribution of deuterium.

Deuterium (2H or D) is a stable and non-radioactive isotope of hydrogen which has approximately twice the mass of protium (H), the most common isotope of hydrogen.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also, unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

In certain embodiments, deuterium enrichment of the compound provided herein is no less than 50%, 60%, 70%, 80%, 90%, 95%, 96%, 97%, 98% or 99%.

In certain embodiments, deuterium enrichment of the compound provided herein is no more than 99.9%, 99%, 98%, 97%, 96%, 95%, or 90%.

In certain embodiments, deuterium enrichment of the compound provided herein is in a range, such as between 50% and 99.9%; between 50% and 99%; between 50% and 98%; between 50% and 97%; between 50% and 96%; between 50% and 95%; between 50% and 90%; between 60% and 99.9%; between 60% and 99%; between 60% and 98%; between 60% and 97%; between 60% and 96%; between 60% and 95%; between 60% and 90%; between 70% and 99.9%; between 70% and 99%; between 70% and 98%; between 70% and 97%; between 70% and 96%; between 70% and 95%; between 70% and 90%; between 80% and 99.9%; between 80% and 99%; between 80% and 98%; between 80% and 97%; between 80% and 96%; between 80% and 95%; between 80% and 90%; between 90% and 99.9%; between 90% and 99%; between 90% and 98%; between 90% and 97%; between 90% and 96%; between 90% and 95%; between 95% and 99.9%; between 95% and 99%; between 95% and 98%; between 95% and 97%; between 95% and 96%; between 96% and 99.9%; between 96% and 99%; between 96% and 98%; between 96% and 97%; between 97% and 99.9%; between 97% and 99%; between 97% and 98%; between 98% and 99.9%; between 98% and 99%; or between 99% and 99.9%.

In certain embodiments, deuterium enrichment of the compound provided herein is between 90% and 99.9%, preferably between 95% and 99.9%, preferably between 97% and 99%, preferably between 98% and 99%, particularly 98.5%. Overall deuterium enrichment of the compounds of the disclosure can be determined using mass spectroscopy, according to methods known in the art.

The term “deuterium enrichment” as used herein refers to the percentage of incorporation of deuterium at a given position in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods, such as mass spectrometry and nuclear magnetic resonance spectroscopy.

The invention also provides pharmaceutically acceptable salts of the compounds of the invention.

A salt of a compound of this invention is formed between an acid and a basic group of the compound, such as an amino functional group, or a base and an acidic group of the compound, such as a carboxyl functional group.

As used herein, the term “pharmaceutically acceptable salt”, unless otherwise indicated, includes salts that retain the biological effectiveness of the free acids and bases of the specified compound and that are not biologically or otherwise undesirable. Contemplated pharmaceutically acceptable salt forms include, but are not limited to, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, PA, Vol. 2, p. 1457, 1995; “Handbook of Pharmaceutical Salts: Properties, Selection, and Use” by Stahl and Wermuth, Wiley-VCH, Weinheim, Germany, 2002. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. Thus, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as L-glycine, L-lysine, and L-arginine, ammonia, primary, secondary, and tertiary amines, and cyclic amines, such as hydroxyethylpyrrolidine, piperidine, morpholine or piperazine, and inorganic salts derived from sodium, calcium, potassium, magnesium, manganese, iron, copper, zinc, aluminum and lithium.

It is also to be understood that the compounds of present disclosure can exist in unsolvated forms, solvated forms (e.g., hydrated forms), and solid forms (e.g., crystal or polymorphic forms), and the present disclosure is intended to encompass all such forms.

As used herein, the term “solvate” or “solvated form” refers to solvent addition forms that contain either stoichiometric or non-stoichiometric amounts of solvent. Some compounds have a tendency to trap a fixed molar ratio of solvent molecules in the crystalline solid state, thus forming a solvate. If the solvent is water, the solvate formed is a hydrate; and if the solvent is alcohol, the solvate formed is an alcoholate. Hydrates are formed by the combination of one or more molecules of water with one molecule of the substance in which the water retains its molecular state as H2O. Examples of solvents that form solvates include, but are not limited to, water, isopropanol, ethanol, methanol, DMSO, ethyl acetate, acetic acid, and ethanolamine.

As used herein, the terms “crystal form”, “crystalline form”, “polymorphic forms” and “polymorphs” can be used interchangeably, and mean crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

Those of skill in the art will appreciate that compounds of the present disclosure may exist in different tautomeric forms, and all such forms are embraced within the scope of the present disclosure. The term “tautomer” or “tautomeric form” refers to structural isomers of different energies which are interconvertible via a low energy barrier. The presence and concentrations of the isomeric forms will depend on the environment the compound is found in and may be different depending upon, for example, whether the compound is a solid or is in an organic or aqueous solution. By way of examples, proton tautomers (also known as prototropic tautomers) include interconversions via migration of a proton, such as keto-enol, amide-imidic acid, lactam-lactim, imine-enamine isomerizations and annular forms where a proton can occupy two or more positions of a heterocyclic system. Valence tautomers include interconversions by reorganization of some of the bonding electrons. Tautomers can be in equilibrium or sterically locked into one form by appropriate substitution. Compounds of the present disclosure identified by name or structure as one particular tautomeric form are intended to include other tautomeric forms unless otherwise specified.

Synthesis of the compounds provided herein, including pharmaceutically acceptable salts thereof, are illustrated in the synthetic schemes in the examples. The compounds provided herein can be prepared using any known organic synthesis techniques and can be synthesized according to any of numerous possible synthetic routes, and thus these schemes are illustrative only and are not meant to limit other possible methods that can be used to prepare the compounds provided herein. Additionally, the steps in the Schemes are for better illustration and can be changed as appropriate. The embodiments of the compounds in examples were synthesized for the purposes of research and potentially submission to regulatory agencies.

The reactions for preparing compounds of the present disclosure can be carried out in suitable solvents, which can be readily selected by one skilled in the art of organic synthesis. Suitable solvents can be substantially non-reactive with the starting materials (reactants), the intermediates, or products at the temperatures at which the reactions are carried out, e.g. temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by one skilled in the art.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999), in P. Kocienski, Protecting Groups, Georg Thieme Verlag, 2003, and in Peter G. M. Wuts, Greene's Protective Groups in Organic Synthesis, 5th Edition, Wiley, 2014, all of which are incorporated herein by reference in its entirety.

Reactions can be monitored according to any suitable method known in the art. For example, product formation can be monitored by spectroscopic means, such as nuclear magnetic resonance spectroscopy (e.g. ¹H or ¹³C), infrared spectroscopy, spectrophotometry (e.g. UV-visible), mass spectrometry, or by chromatographic methods such as high performance liquid chromatography (HPLC), liquid chromatography-mass spectroscopy (LCMS), or thin layer chromatography (TLC). Compounds can be purified by one skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6(6), 874-883, which is incorporated herein by reference in its entirety), and normal phase silica chromatography.

The structures of the compounds in the examples were characterized by nuclear magnetic resonance (NMR). NMR spectra were acquired on Bruker AVANCE III HD 400 and Bruker AVANCE NEO 300 nuclear magnetic resonance spectrometer, running at 400 MHz and 300 MHz respectively for ¹H. ¹H NMR spectra were recorded at 400 MHz &300 MHz in CHCl₃-d and (CH₃)₂SO-d₆ using residual CHCl₃ (7.26 ppm) and DMSO (2.50 ppm) as the internal standard.

LCMS was performed on an Agilent Technology 1260-6125 (ESI).

HPLC spectra was performed on Agilent Technology 1260 equipment with DAD detector and 1290 equipment with DAD detector.

The known starting materials of the present disclosure can be synthesized by using or according to the known methods in the art, or can be purchased from commercial suppliers. Unless otherwise noted, analytical grade solvents and commercially available reagents were used without further purification.

Unless otherwise specified, the reactions of the present disclosure were all done under a positive pressure of nitrogen or argon or with a drying tube in anhydrous solvents, and the reaction flasks were typically fitted with rubber septa for the introduction of substrates and reagents via syringe. Glassware was oven dried and/or heat dried.

For illustrative purposes, the Examples section below shows synthetic route for preparing the compounds of the present disclosure as well as key intermediates. Those skilled in the art will appreciate that other synthetic routes may be used to synthesize the inventive compounds. Although specific starting materials and reagents are depicted, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### Compositions

The invention also provides pharmaceutical compositions comprising an effective amount of a compound of Formula I and/or Formula Ia (e.g., including any of the formulae herein), or a pharmaceutically acceptable salt of said compound; and a pharmaceutically acceptable carrier and/or adjuvant.

As used herein, the term “pharmaceutical composition” refers to a composition containing the molecules or compounds of the present disclosure in a form suitable for administration to a subject.

As used herein, the term “pharmaceutically acceptable” indicates that the substance or composition is compatible chemically and/or toxicologically, with the other ingredients comprising a formulation, and/or the subjects being treated therewith.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions provided herein can be in any form that allows for the composition to be administered to a subject, including, but not limited to a human, and formulated to be compatible with an intended route of administration.

A variety of routes are contemplated for the pharmaceutical compositions provided herein, and accordingly the pharmaceutical composition provided herein may be supplied in bulk or in unit dosage form depending on the intended administration route. For example, for oral, buccal, and sublingual administration, powders, suspensions, granules, tablets, pills, capsules, gelcaps, and caplets may be acceptable as solid dosage forms, and emulsions, syrups, elixirs, suspensions, and solutions may be acceptable as liquid dosage forms. For injection administration, emulsions and suspensions may be acceptable as liquid dosage forms, and a powder suitable for reconstitution with an appropriate solution as solid dosage forms. For inhalation administration, solutions, sprays, dry powders, and aerosols may be acceptable dosage form. For topical (including buccal and sublingual) or transdermal administration, powders, sprays, ointments, pastes, creams, lotions, gels, solutions, and patches may be acceptable dosage form. For vaginal administration, pessaries, tampons, creams, gels, pastes, foams and spray may be acceptable dosage form.

In some embodiments, the pharmaceutical compositions of the present disclosure may be in a form of formulation for oral administration.

In certain embodiments, the pharmaceutical compositions of the present disclosure may be in the form of tablet formulations. Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case using conventional coating agents and procedures well known in the art.

In certain embodiments, the pharmaceutical compositions of the present disclosure may be in a form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

In certain embodiments, the pharmaceutical compositions of the present disclosure may be in the form of aqueous suspensions, which generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

In certain embodiments, the pharmaceutical compositions of the present disclosure may be in the form of oily suspensions, which generally contain suspended active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

In certain embodiments, the pharmaceutical compositions of the present disclosure may be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

In certain embodiments, the pharmaceutical compositions provided herein may be in the form of syrups and elixirs, which may contain sweetening agents such as glycerol, propylene glycol, sorbitol, aspartame or sucrose, a demulcent, a preservative, a flavoring and/or coloring agent.

In some embodiments, the pharmaceutical compositions of the present disclosure may be in a form of formulation for injection administration.

In certain embodiments, the pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables.

In some embodiments, the pharmaceutical compositions of the present disclosure may be in a form of formulation for inhalation administration.

In certain embodiments, the pharmaceutical compositions of the present disclosure may be in the form of aqueous and nonaqueous (e.g., in a fluorocarbon propellant) aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols.

In some embodiments, the pharmaceutical compositions of the present disclosure may be in a form of formulation for topical or transdermal administration.

In certain embodiments, the pharmaceutical compositions provided herein may be in the form of creams, ointments, gels and aqueous or oily solutions or suspensions, which may generally be obtained by formulating an active ingredient with a conventional, topically acceptable excipients such as animal and vegetable fats, oils, waxes, paraffins, starch, tragacanth, cellulose derivatives, polyethylene glycols, silicones, bentonites, silicic acid, talc and zinc oxide, or mixtures thereof.

In certain embodiments, the pharmaceutical compositions provided herein may be formulated in the form of transdermal skin patches that are well known to those of ordinary skill in the art.

Besides those representative dosage forms described above, pharmaceutically acceptable excipients and carriers are generally known to those skilled in the art and are thus included in the present disclosure. Such excipients and carriers are described, for example, in “Remingtons Pharmaceutical Sciences” Mack Pub. Co., New Jersey (1991), in “Remington: The Science and Practice of Pharmacy”, Ed. University of the Sciences in Philadelphia, 21^{st} Edition, LWW (2005), which are incorporated herein by reference.

In some embodiments, the pharmaceutical compositions of the present disclosure can be formulated as a single dosage form. The amount of the compounds provided herein in the single dosage form will vary depending on the subject treated and particular mode of administration.

In some embodiments, the pharmaceutical compositions of the present disclosure can be formulated as short-acting, fast-releasing, long-acting, and sustained-releasing. Accordingly, the pharmaceutical formulations of the present disclosure may also be formulated for controlled release or for slow release.

In another embodiment, a composition of this invention further comprises a second therapeutic agent. The second therapeutic agent may be selected from any compound or therapeutic agent known to have or that demonstrates advantageous properties when administered with a compound having the same mechanism of action as the compound of this invention.

In another embodiment, the invention provides separate dosage forms of a compound of this invention and one or more of any of the second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In some embodiments, the second therapeutic agent can include: (1) a cholesterol absorption inhibitor, (2) an HMG-COA reductase inhibitor, (3) a bile acid sequestrant, (4) nicotinyl alcohol, nicotinic acid or a salt thereof, (5) a phenolic anti-oxidant, (6) an ACAT inhibitor, and (7) a CTEP inhibitor.

In the pharmaceutical compositions of the invention, the compound of the present invention is present in an effective amount. As used herein, the term “effective amount” refers to an amount which, when administered in a proper dosing regimen, is sufficient to treat the target disorder.

The interrelationship of dosages for animals and humans (based on milligrams per meter squared of body surface) is described in Freireich et al., Cancer Chemother. Rep, 1966, 50: 219. Body surface area may be approximately determined from height and weight of the subject. See, e.g., Scientific Tables, Geigy Pharmaceuticals, Ardsley, N.Y., 1970, 537.

In some embodiments, an effective amount of a compound of this invention can range from about 0.5 μg per day to about 90 mg per day, 1 μg per day to about 50 mg per day, 2 μg per day to about 10 mg per day, 3 μg per day to about 1 mg per day, 5 μg per day to about 800 μg per day, 5 μg per day to about 600 μg per day, 5 μg per day to about 500 μg per day, 10 μg per day to about 500 μg per day, 12 μg per day to about 500 μg per day, 15 μg per day to about 500 μg per day, 20 μg per day to about 500 μg per day, 25 μg per day to about 500 μg per day. In some embodiments, an effective amount of a compound of this invention can range from about 25 μg per day to about 300 μg per day. In some embodiments, an effective amount of a compound of this invention can range from about 50 μg per day to about 150 μg per day.

Effective doses will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician. For example, guidance for selecting an effective dose can be determined by reference to the prescribing information for aleglitazar.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another aspect, the present disclosure provides a dual agonist of PPARα and PPARγ for use in a method for the treatment and/or prophylaxis of diseases which are modulated by PPARα and/or PPARγ agonists, wherein the dual agonist of PPARα and PPARγ is deuterated.

In another aspect, the present disclosure provides a method for the treatment and/or prophylaxis of diseases which are modulated by PPARα and/or PPARγ agonists in a subject, comprising administering a dual agonist of PPARα and PPARγ to the subject, wherein the dual agonist of PPARα and PPARγ is deuterated.

In another aspect, the present disclosure provides the use of a dual agonist of PPARα and PPARγ in the manufacture of a medicament for the treatment and/or prophylaxis of diseases which are modulated by PPARα and/or PPARγ agonists, wherein the dual agonist of PPARα and PPARγ is deuterated.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, etc. The terms “subject” and “patient” are used interchangeably herein in reference, for example, to a mammalian subject, such as a human patient.

The terms “treat”, “treating” and “treatment” are meant to include improving, preventing, alleviating or abrogating a disorder; or alleviating, preventing or abrogating one or more of the symptoms associated with the disorder; and/or preventing, alleviating or eradicating the cause(s) of the disorder itself, i.e., causing a clinical symptom to not significantly develop in a mammal that may be predisposed to the disease but does not yet experience or display symptoms of the disease. This may include improving the subject's ability to perform activities of daily living, perform domestic chores, manage finances, and/or perform an occupation or reduce the level of care needed by the subject. Treat, treating or treatment may include improvement of the symptom by at least 20%, 30%, 50%, 80%, 90%, or by 100%. Symptoms associated with a specific disorder depend on the specific disorder at hand.

The term “administering” means either directly administering a compound or composition of the present invention, or administering a prodrug, derivative or analog which will form an equivalent amount of the active compound or substance within the body.

The term “disease” means any condition or disorder that damages or interferes with the normal function of a cell, tissue, or organ.

In certain embodiments, the disease is diabetes, non-insulin dependent diabetes mellitus, elevated blood pressure, dyslipidemia, atherosclerotic diseases, metabolic syndrome, or diabetic nephropathy.

In certain embodiments, the disease is non-insulin dependent diabetes mellitus, or diabetic nephropathy.

In certain embodiments, the disease is diabetic nephropathy.

The term “diabetes” as used herein means a disease in which a patient's ability to control glucose levels in blood is impaired, because of the partially lost the ability to respond properly to the action of insulin.

The term “non-insulin dependent diabetes mellitus” as used herein also referred to type II diabetes (T2D), which afflicts 80-90% of all diabetic patients in developed countries, the Isles of Langerhans in the pancreas still produce insulin. However, the target organs, mainly muscle, liver and adipose tissue, exhibit a profound resistance to insulin stimulation, and the body compensates by producing unphysiologically high levels of insulin. In later stage of disease, however, insulin secretion decreases due to exhaustion of the pancreas.

The term “atherosclerotic disease” as used herein, also known as arteriosclerotic vascular disease or ASVD, is a specific form of arteriosclerosis in which an artery wall thickens as a result of invasion and accumulation of white blood cells (foam cells) and proliferation of intimal-smooth-muscle cell creating an atheromatous (fibrofatty) plaque.

The term “metabolic syndrome” as used herein means a cluster of conditions that occur together, increasing the risk of heart disease, stroke and type 2 diabetes. These conditions include increased blood pressure, high blood sugar, excess body fat around the waist, and abnormal cholesterol or triglyceride levels.

The term “diabetic nephropathy” as used herein means a kidney disease that results from diabetes, which is the number one cause of kidney failure. Almost a third of people with diabetes develop diabetic nephropathy. There are often no symptoms with early diabetic nephropathy. As the kidney function worsens, symptoms may include: swelling of the hands, feet, and face; trouble sleeping or concentrating; poor appetite; nausea; weakness; itching (end-stage kidney disease) and extremely dry skin; drowsiness (end-stage kidney disease); abnormalities in the hearts' regular rhythm, because of increased potassium in the blood; and muscle twitching.

In certain embodiments, the disease is renal injury. In certain embodiments, the renal injury is caused by ureteral obstruction. In certain embodiments, the renal injury is caused by unilateral ureteral obstruction.

In certain embodiments, the dual agonist of PPARα and PPARγ is deuterated Aleglitazar or its pharmaceutical acceptable salt.

The term “aleglitazar” as used herein, also known as RG-1439 or RO-0728804, is a dual agonist of peroxisome proliferator-activated receptors α/γ (PPARα/γ) with insulin-sensitizing and glucose-lowering actions and favorable effects on lipid profiles. It is investigated for use in patients with type II diabetes to reduce their risks of cardiovascular mortality and morbidity. “Aleglitazar” has the structure as follows:

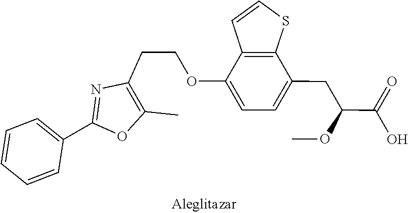 [SMILES: CO[C@@H](Cc1ccc(OCCc2nc(-c3ccccc3)oc2C)c2ccsc12)C(=O)O]

The term “PPARα/γ dual agonist” as used herein, refers to compounds that exhibit both significant PPARα and PPARγ agonism. In some embodiments, PPARα/γ dual agonist exhibits significant PPARα and/or PPARγ agonism, wherein the half-maximal concentration potencies (EC₅₀) for activation of hPPARγ and EC₅₀ for activation of hPPARα differ by less than 30-fold, 25-fold, 20-fold, 15-fold, 10-fold, 5-fold, or 3-fold. In some embodiments, PPARα/γ dual agonist exhibits significant PPARα and/or PPARγ agonism, wherein the half-maximal concentration potencies (EC₅₀) for activation of hPPARγ and EC₅₀ for activation of hPPARα differ by more than 30-fold, 25-fold, 20-fold, 15-fold, 10-fold, 5-fold, or 3-fold.

In certain embodiments, the dual agonist of PPARα and PPARγ is the compound or its pharmaceutical acceptable salt provided herein.

In another aspect, the present disclosure provides a method for modulating the specific agonistic activity of PPARα or PPARγ of a dual agonist of PPARα and PPARγ, comprising deuterating the agonist.

In another aspect, the present disclosure provides a method for improving the specific agonistic activity of PPARα or PPARγ of a dual agonist of PPARα and PPARγ, comprising deuterating the agonist.

In certain embodiments, the specific agonistic activity of PPARα of the agonist is improved.

In certain embodiments, the specific agonistic activity of PPARγ of the agonist is improved.

In certain embodiments, at least one, two, three, four, five, six, seven, eight, nine, ten of H of the agonist is deuterated.

In certain embodiments, no more than one, two, three, four, five, six, seven, eight, nine, ten of H of the agonist is deuterated.

In certain embodiments, the dual agonist of PPARα and PPARγ is Aleglitazar or its pharmaceutical acceptable salt.

It is to be understood that both the foregoing general description and the following detailed description are exemplary and explanatory only and are not restrictive of the invention as claimed. In this application, the use of “or” means “and/or” unless stated otherwise. Furthermore, the use of the term “including”, as well as other forms, such as “includes” and “included”, is not limiting. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1: Synthesis of Compound 1

### Reaction Scheme

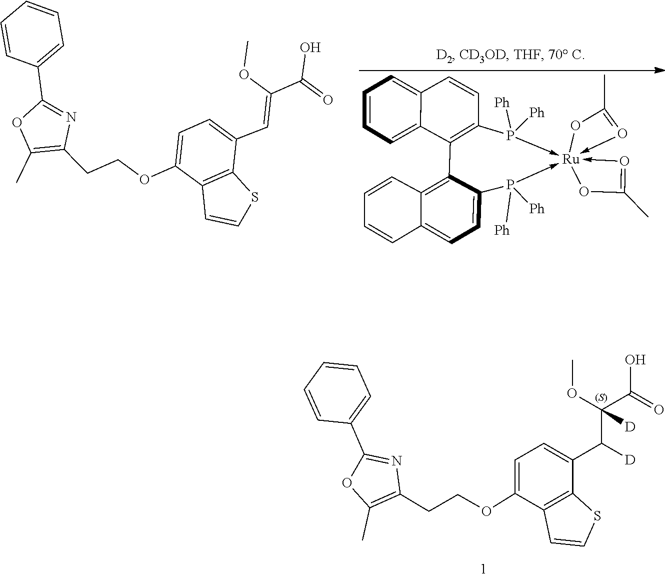

### Process Description

A 300 mL hydrogenation reactor was charged (Z)-2-methoxy-3-(4-(2-(5-methyl-2-phenyloxazol-4-yl)ethoxy)benzo[b]thiophen-7-yl)acrylic acid (2.3 g, 5.3 mmol), (S)-phenylethylamine (230 mg, 1.9 mmol), CD₃OD (24 mL), THF (16 mL) and Ru-cat (CAS: 261948-85-0, 46 mg). The reaction mixture was stirred at 70° C. under 30 bar of D₂ for 1 day. After the autoclave was opened and the yellowish solution was rotary evaporated to dryness (45° C.). The crude product was dissolved in EtOAc (200 mL) and washed with 1 N HCl (60 mL×2). The organic layer was dried over Na₂SO₄, filtered and evaporated to dryness. The crude product was dissolved in isopropyl acetate at reflux and allowed to cool to 0° C. whereby crystallization started. The formed crystals were filtered off, washed with isopropyl acetate (50 mL) and dried to afford light yellow solid (940 mg, ˜ 60% ee), which was separated by chiral HPLC (OJ-H(OJHOCD-WB010)), eluting with 0.1% HCOOH in MeOH) and further purified by prep-HPLC (0.1% FA in CH₃CN and water) to give compound 1 (610 mg, yield 26.2%) as a white solid.

¹H NMR (400 MHZ, CDCl₃) δ: 7.97 (dd, J=6.4, 2.4 Hz, 2H), 7.48 (d, J=5.5 Hz, 1H), 7.43-7.41 (m, 3H), 7.32 (d, J=5.5 Hz, 1H), 7.15 (d, J=8.0 Hz, 1H), 6.74 (d, J=8.0 Hz, 1H), 4.35 (t, J=6.5 Hz, 2H), 3.34 (s, 3H), 3.19 (s, 1H), 3.06 (t, J=6.5 Hz, 2H), 2.40 (s, 3H).

LC-MS (ESI⁺): m/z=440.2 ([M+H]+).

Chiral HPLC (Chiralpak AD-3 4.6 mm*250 mm 3 μm, 90% hexanes/9.99% EtOH/0.01% TFA, 210 nm): 99.99% ee.

### Example 2: Synthesis of Compound 2

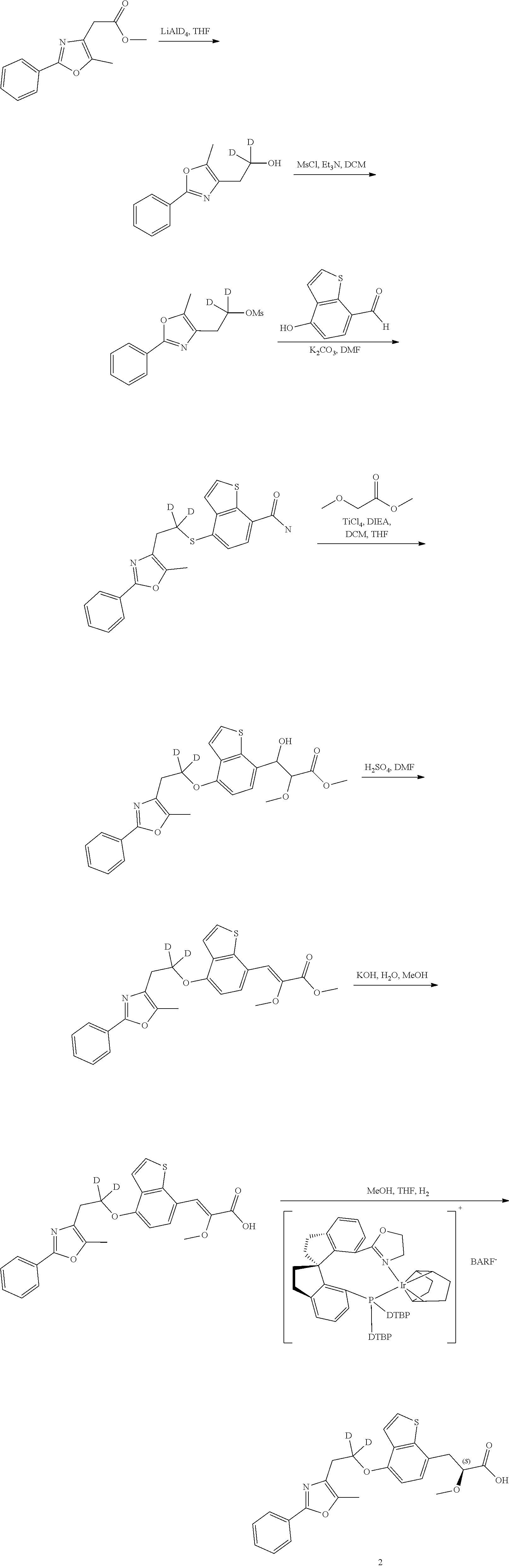

To a suspension of LiAID₄ (1.9 g, 45.4 mmol) in THF (40 mL) was added methyl 2-(5-methyl-2-phenyloxazol-4-yl)acetate (7.0 g, 30.3 mmol) in THF (60 mL) at 0° C. under N₂. The reaction was stirred at 0° C. for 2 hours, then quenched with water (3 mL). The resulting solid was filtered off. The filter cake was washed with EtOAc (500 mL), DCM/MeOH (10/1, 500 mL). The filtrate was concentrated under vacuum to afford 2-(5-methyl-2-phenyloxazol-4-yl)ethan-1,1-d₂-1-ol (5.0 g, yield 80.6%) as a yellow solid.

¹H NMR (400 MHZ, CDCl₃) δ: 8.05-7.87 (m, 1H), 7.50-7.33 (m, 2H), 2.71 (s, 2H), 2.34 (s, 3H).

LC-MS (ESI⁺): m/z=206.2 ([M+H]⁺).

To a solution of 2-(5-methyl-2-phenyloxazol-4-yl)ethan-1,1-d₂-1-ol (5.0 g, 24.4 mmol) in DCM (100 mL) was added Et₃N (5.4 g, 53.7 mmol). The reaction mixture was cooled to 0° C., MsCl (5.6 g, 48.8 mmol) was added under N₂. The reaction was stirred at 0° C. for 2 hours, then poured into water. 1 N HCl (40 mL) was added and the mixture was extracted with DCM (100 mL×2). The combined organic layer was dried over Na₂SO₄, filtered and concentrated under vacuum. The residue was purified by column chromatography (Petroleum ether: EtOAc=20:1 to 10:1) to give 2-(5-methyl-2-phenyloxazol-4-yl)ethyl-1,1-d₂ methanesulfonate (5.0 g, yield 72.5%) as a white solid.

¹H NMR (400 MHZ, CDCl₃) δ: 7.97 (dd, J=7.4, 2.2 Hz, 2H), 7.53-7.34 (m, 3H), 3.04-2.90 (m, 5H), 2.36 (s, 3H).

LC-MS (ESI⁺): m/z=284.0 ([M+H]⁺).

To a solution of 4-hydroxybenzo[b]thiophene-7-carbaldehyde (3.2 g, 17.7 mmol) in DMF (30 mL) was added K₂CO₃ (2.9 g, 21.2 mmol). The reaction mixture was heated to 85° C. under N₂. 2-(5-methyl-2-phenyloxazol-4-yl)ethyl-1,1-d₂ methanesulfonate (5.0 g, 17.7 mmol) in DMF (15 mL) was added dropwise at this temperature. The reaction was stirred at 85° C. for 5 hours, then it was cooled to room temperature and poured into water, extracted with EtOAc (300 mL×2). The organic layer was washed with brine, dried over Na₂SO₄, filtered and concentrated. The residue was triturated with Petroleum ether/EtOAc=5/1 to give 4-(2-(5-methyl-2-phenyloxazol-4-yl)ethoxy-1,1-d₂)benzo[b]thiophene-7-carbaldehyde (5.7 g, yield 88.2%) as a brown solid.

¹H NMR (300 MHZ, DMSO-d₆) δ: 10.05 (s, 1H), 8.07 (d, J=8.2 Hz, 1H), 8.00-7.86 (m, 2H), 7.82 (d, J=5.5 Hz, 1H), 7.62-7.38 (m, 4H), 7.23 (d, J=8.1 Hz, 1H), 3.06 (s, 2H), 2.40 (s, 3H).

LC-MS (ESI⁺): m/z=366.0 ([M+H]⁺).

To a solution of methyl 2-methoxyacetate (5.9 g, 57.2 mmol) in THF (40 mL) was added TiCl₄ (10.8 g, 57.2 mmol) at 0° C. under argon. The yellow solution was stirred at 0° C. for 15 min and DIEA (7.9 g, 61.6 mmol) was added. The black solution was stirred for another 15 min and 4-(2-(5-methyl-2-phenyloxazol-4-yl)ethoxy-1,1-d₂)benzo[b]thiophene-7-carbaldehyde (4.0 g, 11.0 mmol) in DCM (60 mL) was added dropwise. The reaction was stirred at 0° C. for 1 hour and warmed to room temperature overnight. Then it was cooled to 0° C. and quenched with water, extracted with DCM (200 mL×2). The organic layer was washed with brine, dried over Na₂SO₄, filtered and concentrated to give the crude methyl 3-hydroxy-2-methoxy-3-(4-(2-(5-methyl-2-phenyloxazol-4-yl)ethoxy-1,1-d₂)benzo[b]thiophen-7-yl)propanoate (7.7 g), which was directly used to the next step without further purification.

LC-MS (ESI⁺): m/z=470.2 ([M+H]⁺).

To a solution of methyl 3-hydroxy-2-methoxy-3-(4-(2-(5-methyl-2-phenyloxazol-4-yl)ethoxy-1,1-d₂)benzo[b]thiophen-7-yl)propanoate (7.7 g, crude) in DMF (40 mL) was added con. H₂SO₄ (10 mL) dropwise at ambient temperature. The reaction was stirred at 100° C. overnight, then it was diluted with EtOH (40 mL) and stirred at 0° C. for 1 hour. The solid was filtered, washed with EtOH (10 mL) and water (50 mL). The wet cake was dried to give methyl (Z)-2-methoxy-3-(4-(2-(5-methyl-2-phenyloxazol-4-yl)ethoxy-1,1-d₂)benzo[b]thiophen-7-yl)acrylate (2.3 g, 46.4% yield of 2 steps) as a yellow solid.

¹H NMR (400 MHZ, CDCl₃) δ: 8.09 (d, J=8.4 Hz, 1H), 7.99 (dd, J=7.6, 1.8 Hz, 2H), 7.53-7.37 (m, 4H), 7.34 (d, J=5.5 Hz, 1H), 7.21 (s, 1H), 6.85 (d, J=8.4 Hz, 1H), 3.88 (s, 3H), 3.77 (s, 3H), 3.08 (s, 2H), 2.40 (s, 3H).

LC-MS (ESI⁺): m/z=452.2 ([M+H]⁺).

To a solution of methyl (Z)-2-methoxy-3-(4-(2-(5-methyl-2-phenyloxazol-4-yl)ethoxy-1,1-d₂)benzo[b]thiophen-7-yl)acrylate (2.3 g, 5.1 mmol) in MeOH (50 mL) was added KOH (1.7 g, 30.6 mmol) in H₂O (5 mL) at room temperature. The reaction was stirred at 80° C. for 2 hours. The reaction mixture was cooled to room temperature, diluted with H₂O (50 mL) and adjusted pH=3 with 6 N HCl. The mixture was cooled to 0° C. and the solid was filtered. The filter cake was suspended in EtOH (40 mL) at 80° C. for 1 hour, cooled to 0° C. and stirred for 1 hour. The solid was filtered and dried to afford (Z)-2-methoxy-3-(4-(2-(5-methyl-2-phenyloxazol-4-yl)ethoxy-1,1-d₂)benzo[b]thiophen-7-yl)acrylic acid (1.5 g, yield 68.2%) as a brown solid.

¹H NMR (400 MHZ, CDCl₃) δ: 8.09 (d, J=8.4 Hz, 1H), 8.00 (dd, J=7.6, 1.9 Hz, 2H), 7.49 (d, J=5.5 Hz, 1H), 7.48-7.39 (m, 3H), 7.35 (t, J=2.7 Hz, 2H), 6.87 (d, J=8.4 Hz, 1H), 3.78 (s, 3H), 3.10 (s, 2H), 2.41 (s, 3H).

LC-MS (ESI⁺): m/z=438.2 ([M+H]⁺).

A 300 mL stainless steel autoclave was charged with (Z)-2-methoxy-3-(4-(2-(5-methyl-2-phenyloxazol-4-yl)ethoxy-1,1-d₂)benzo[b]thiophen-7-yl)acrylic acid (1.5 g, 3.4 mmol), (S)-phenylethylamine (82 mg, 0.68 mmol), MeOH (18 mL), THF (12 mL) and Ir-cat ([(S)-DTBSIPHOX)Ir(COD)]BArF, 9.3 mg, 0.001 eq). The autoclave was sealed and the hydrogenation was stirred at 70° C. under 30 bar of hydrogen for 16 hours. LCMS showed about half of starting material was remained, Ir-cat (10.3 mg) was added and the reaction was stirred for another 1 day. After the autoclave was opened and the yellowish solution was rotary evaporated to dryness (45° C.). The crude product was dissolved in EtOAc (150 mL) and was washed with 1 N HCl (40 mL×2). The organic layer was dried over Na₂SO₄, filtered and evaporated to dryness. The crude product was dissolved in isopropyl acetate at reflux and allowed to cool to 0° C. whereby crystallization started. The formed crystals were filtered off, washed with isopropyl acetate (50 mL) and dried to afford a yellow solid (920 mg), which was further purified by prep-HPLC (0.1% FA in CH₃CN and water) to give compound 2 (518 mg, yield 34.5%) as a white solid.

¹H NMR (400 MHZ, CDCl₃) δ: 7.99 (dd, J=6.4, 2.4 Hz, 2H), 7.48 (d, J=5.6 Hz, 1H), 7.43-7.41 (m, 3H), 7.32 (d, J=5.6 Hz, 1H), 7.15 (d, J=8.0 Hz, 1H), 6.73 (d, J=8.0 Hz, 1H), 4.20 (dd, J=7.9, 4.7 Hz, 1H), 3.39-3.28 (m, 4H), 3.23-3.18 (m, 1H), 3.05 (s, 2H), 2.40 (s, 3H).

LC-MS (ESI⁺): m/z=440.2 ([M+H]⁺).

Chiral HPLC (Chiralpak AD-3 4.6 mm*250 mm 3 μm, 90% hexanes/9.99% EtOH/0.01% TFA, 210 nm): 99.57% ee.

### Example 3: Synthesis of Compound 3

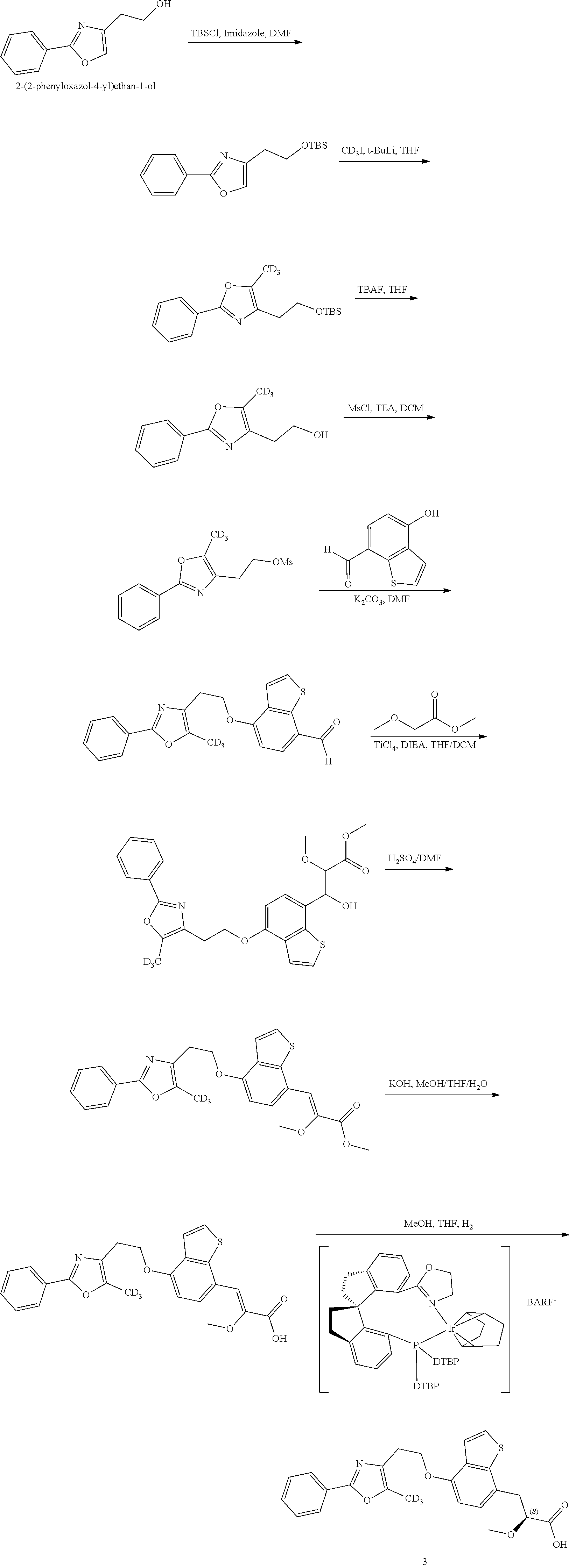

### Process Description

To a solution of 2-(2-phenyloxazol-4-yl)ethan-1-ol (22.7 g, 120.0 mmol, 1.0 eq) in DMF (230 mL) was added imidazole (24.5 g, 360.0 mmol, 3.0 eq) and t-butyldimethylsilyl chloride (27.1 g, 180.0 mmol, 1.5 eq) portion wise. The mixture was stirred at room temperature for 1 hour. After the reaction completed, the reaction mixture was diluted with EtOAc (100 mL), washed with H₂O (100 mL×2) and brine (100 mL×2). The organic phase was dried over Na₂SO₄, filtered and concentrated to give a crude product, which was purified by silica gel column (eluted with petroleum ether/EtOAc=30:1) to afford 4-(2-((tert-butyldimethylsilyl)oxy)ethyl)-2-phenyloxazole (30.0 g, yield 82.3%) as a colorless oil.

¹HNMR (400 MHz, CDCl₃) δ: 8.03-8.01 (m, 2H), 7.50 (s, 1H), 7.47-7.42 (m, 3H), 3.92 (t, J=6.8 Hz, 2H), 2.83-2.80 (m, 2H), 0.88 (s, 9H), 0.03 (s, 6H).

LC-MS (ESI⁺):304.1 ([M+H]⁺).

Under argon, a solution of 4-(2-((tert-butyldimethylsilyl)oxy)ethyl)-2-phenyloxazole (30.0 g, 99.0 mmol, 1.0 eq) in THF (300 mL) was cooled to −78° C. and then t-BuLi (1 M, 114 mL, 148.0 mmol, 1.5 eq) was added dropwise. The mixture was warmed to −40° C. and stirred for 1 hour. After that the mixture was cooled to −78° C. again, CD₃I (28.7 g, 198.0 mmol, 2.0 eq) was added dropwise. The reaction mixture was stirred at this temperature for 1 hour, then warmed to −40° C. and stirred for another 1 hour. The reaction was quenched with saturated aq. NH₄Cl (300 mL), extracted with EtOAc (300 mL×2). The organic phase was dried over Na₂SO₄, filtered and concentrated to afford a crude residue, which was purified by silica gel column (eluted with ether/EtOAc=50:1) to afford 4-(2-((tert-butyldimethylsilyl)oxy)ethyl)-5-(methyl-d₃)-2-phenyloxazole (11.0 g, yield 35.6%) as a yellow oil.

¹HNMR (400 MHZ, CDCl₃) δ: 7.98 (dd, J=7.6, 1.6 Hz, 2H), 7.44-7.39 (m, 3H), 3.89 (t, J=6.8 Hz, 2H), 2.71 (t, J=6.8 Hz, 2H), 0.87 (s, 9H), 0.00 (s, 6H).

LC-MS (ESI⁺): 321.2 ([M+H]⁺).

Under argon, to a solution of 4-(2-((tert-butyldimethylsilyl)oxy)ethyl)-5-(methyl-d₃)-2-phenyloxazole (10.0 g, 31.1 mmol, 1.0 eq) in THF (100 mL) was added TBAF (1 M in THF, 62.2 mL, 62.2 mmol, 2.0 eq) dropwise at 0° C., then the resulting mixture was stirred for 1 hour at room temperature. The reaction mixture was diluted with EtOAc (100 mL), washed with saturated NH₄Cl (100 mL×2) and brine (100 mL). The organic phase was dried over Na₂SO₄, filtered and concentrated to afford the crude product, which was purified by silica gel column (eluted with petroleum ether/EtOAc=2:1) to afford 2-(5-(methyl-d₃)-2-phenyloxazol-4-yl)ethan-1-ol (5.7 g, yield 88.7%) as a colorless oil.

¹HNMR (400 MHZ, CDCl₃) δ: 8.00-7.97 (m, 2H), 7.46-7.41 (m, 3H), 3.93 (t, J=6.0 Hz, 2H), 2.90 (brs, 1H), 2.73 (t, J=6.0 Hz, 2H).

LC-MS (ESI⁺): 207.1 ([M+H]⁺).

To a solution of 2-(5-(methyl-d₃)-2-phenyloxazol-4-yl)ethan-1-ol (3.5 g, 16.9 mmol, 1.0 eq) in dichloromethane (32.5 mL) was added triethylamine (3.8 g, 37.2 mmol, 2.2 eq) at room temperature. The mixture was cooled to 0° C. and methanesulfonyl chloride (3.9 g, 33.8 mmol, 2.0 eq) was added dropwise over 10 min. The reaction was stirred at 5° C. for 2 hours. The reaction mixture was quenched with 1 N HCl (10 mL), extracted with dichloromethane (20 mL×2). The combined organic layer was washed with aqueous NaHCO₃ (20 mL×2) and brine (20 mL×2), dried with Na₂SO₄, filtered and concentrated under vacuum to afford 2-(5-(methyl-d₃)-2-phenyloxazol-4-yl)ethyl methanesulfonate (4.5 g, crude) as a yellow solid, which was directly used to the next step without further purification.

¹HNMR (400 MHZ, CDCl₃) δ: 7.99 (dd, J=7.6, 2.7 Hz, 2H), 7.46-7.44 (m, 3H), 4.54 (t, J=6.8 Hz, 2H), 2.99-2.96 (m, 5H).

LC-MS (ESI⁺): 285.2 ([M+H]⁺).

Under argon, to a solution of 4-hydroxybenzo[b]thiophene-7-carbaldehyde (2.8 g, 15.7 mmol, 1.0 eq) in N,N-dimethylformamide (35 mL) was added K₂CO₃ (2.6 g, 18.8 mmol, 1.2 eq) at room temperature. The reaction mixture was heated to 85° C. and then a solution of 2-(5-(methyl-d₃)-2-phenyloxazol-4-yl)ethyl methanesulfonate (4.5 g, 15.7 mmol, 1.0 eq) in DMF (20 mL) was added. The reaction mixture was stirred for 5 hours. Then the reaction mixture was poured into water (150 mL), extracted with EtOAc (150 mL×2). The organic layer was washed with water (100 mL×2), brine (100 mL×2). The organic layer was dried over Na₂SO₄, filtered and concentrated to give a crude product, which was washed with EtOAc to afford 4-(2-(5-(methyl-d₃)-2-phenyloxazol-4-yl)ethoxy)benzo[b]thiophene-7-carbaldehyde (4.1 g, yield 71.1%) as a white solid. The crude product was used for next step without further purification.

¹HNMR (400 MHZ, CDCl₃) δ: 10.06 (s, 1H), 8.00-7.97 (m, 2H), 7.81 (d, J=8.0 Hz, 1H), 7.56 (s, 2H), 7.46-7.40 (m, 3H), 6.95 (d, J=8.0 Hz, 1H), 4.53 (t, J=6.4 Hz, 2H), 3.13 (t, J=6.4 Hz, 2H).

LC-MS (ESI⁺): 367.0 ([M+H]⁺).

Under argon, to a solution of methyl 2-methoxyacetate (5.9 g, 57.0 mmol, 5.2 eq) in THF (65 mL) was added TiCl₄ (10.7 g, 57.0 mmol, 5.2 eq) dropwise at 0° C. After the yellow solution was stirred for 15 min, DIEA (7.86 g, 61 mmol, 5.6 eq) was added. The solution was stirred for 15 min, a solution of 4-(2-(5-(methyl-d₃)-2-phenyloxazol-4-yl)ethoxy)benzo[b]thiophene-7-carbaldehyde (4.1 g, 11 mmol, 1.0 eq) in dichloromethane (65 mL) was added dropwise. After stirred for 60 min the reaction mixture was allowed to warm up to 20° C. and stirred overnight. The reaction mixture was cooled to 0° C. and quenched with ice water (150 mL). The organic layer was separated, the aqueous layer was extracted with DCM (50 mL×2). The combined organic layer was washed with water (50 mL×2), dried over Na₂SO₄, filtered and evaporated to dryness to give methyl 3-hydroxy-2-methoxy-3-(4-(2-(5-(methyl-d₃)-2-phenyloxazol-4-yl)ethoxy)benzo[b]thiophen-7-yl)propanoate (crude, 6.2 g) as an orange oil, which was directly used to the next step without further purification.

LC-MS (ESI⁺): 471.2 ([M+H]⁺).

To a solution of methyl 3-hydroxy-2-methoxy-3-(4-(2-(5-(methyl-d₃)-2-phenyloxazol-4-yl)ethoxy)benzo[b]thiophen-7-yl)propanoate (crude, 6.2 g) in DMF (100 mL) was added con. H₂SO₄ (25 mL). The resulting dark brown solution was stirred at 100° C. overnight. The reaction solution was cooled to room temperature, poured into ice water (100 mL) and extracted with EtOAc (100 ml×2). The combined organic layer was washed with water (100 mL×2) and brine (100 mL×2). The organic layer was concentrated to afford a crude product, which was purified by silica gel column (eluted with petroleum ether/EtOAc=3:1) to afford methyl (Z)-2-methoxy-3-(4-(2-(5-(methyl-d₃)-2-phenyloxazol-4-yl)ethoxy)benzo[b]thiophen-7-yl)acrylate (1.5 g, yield 30.1% for two steps) as an oil.

¹HNMR (400 MHZ, DMSO-d₆) δ: 8.03 (d, J=8.4 Hz, 1H), 7.92-7.89 (m, 2H), 7.70 (d, J=5.6 Hz, 1H), 7.52-7.46 (m, 3H), 7.43 (d, J=5.6 Hz, 1H), 7.06 (d, J=8.4 Hz, 1H), 7.00 (s, 1H), 4.43 (t, J=6.4 Hz, 2H), 3.81 (s, 3H), 3.72 (s, 3H), 3.04 (t, J=6.4 Hz, 2H).

LC-MS (ESI⁺): 453.2 ([M+H]⁺).

To a solution of methyl (Z)-2-methoxy-3-(4-(2-(5-(methyl-d₃)-2-phenyloxazol-4-yl)ethoxy)benzo[b]thiophen-7-yl)acrylate (1.5 g, 3.4 mmol, 1.0 eq) in MeOH (30 mL) was added a solution of KOH (1.14 g, 20.2 mmol, 6.0 eq) in water (3 mL). The suspension was stirred for 1.5 hours at 60° C. The formed yellowish reaction solution was cooled to room temperature, adjust pH to 3˜4 with 1N HCl and extracted with EtOAc (30 mL×2). The organic phase was dried over Na₂SO₄, filtered and concentrated to afford the crude product which was triturated with EtOAc and filtered to give (Z)-2-methoxy-3-(4-(2-(5-(methyl-d₃)-2-phenyloxazol-4-yl)ethoxy)benzo[b]thiophen-7-yl)acrylic acid (1.1 g, yield 73.8%) as a white solid.

¹HNMR (400 MHZ, CDCl₃) δ: 8.10 (d, J=8.4 Hz, 1H), 8.00-7.98 (m, 2H), 7.47 (d, J=5.6 Hz, 1H), 7.46-7.41 (m, 3H), 7.36-7.34 (m, 2H), 6.87 (d, J=8.4 Hz, 1H), 4.47 (t, J=6.4 Hz, 2H), 3.79 (s, 3H), 3.11 (t, J=6.4 Hz, 2H).

LC-MS (ESI⁺): 439.0 ([M+H]⁺).

A 30 mL stainless steel autoclave was charged with (Z)-2-methoxy-3-(4-(2-(5-(methyl-d₃)-2-phenyloxazol-4-yl)ethoxy)benzo[b]thiophen-7-yl)acrylic acid (300.0 mg, 0.68 mmol, 1.0 eq), (S)-phenylethylamine (16.6 mg, 0.14 mmol, 0.2 eq), MeOH (3.6 mL), THF (2.4 mL) and Ir-cat ([((S)-DTBSIPHOX)Ir(COD)]BArF, 2.4 mg, 0.002 eq). The autoclave was sealed, and the hydrogenation was stirred at 70° C. under 30 bar of hydrogen for 36 hours. The reaction solution was evaporated to dryness. The crude product was dissolved in DCM (20 mL) and washed with 1N HCl (10 mL). The organic layer was dried over Na₂SO₄, filtered and concentrated to afford a crude product, which was purified by Prep-HPLC to give compound 3 (200 mg, yield 66.1%) as a white solid.

¹HNMR (400 MHZ, CDCl₃) δ: 7.97 (dd, J=8.0, 2.8 Hz, 1H), 7.47 (d, J=5.6 Hz, 1H), 7.44-7.40 (m, 3H), 7.31 (d, J=5.6 Hz, 1H), 7.15 (d, J=8.0 Hz, 1H), 6.72 (d, J=8.0 Hz, 1H), 4.34 (t, J=6.4 Hz, 2H), 4.21-4.18 (m, 1H), 3.36-3.32 (m, 4H), 3.24-3.18 (m, 1H), 3.06 (t, J=6.4 Hz, 2H).

LC-MS (ESI⁺): 441.1 ([M+H]⁺).

Chiral HPLC (Chiralpak AD-3 4.6 mm*250 mm 3 μm, 90% hexanes/9.99% EtOH/0.01% TFA, 210 nm): 99.0% ee.

### Example 4: Synthesis of Compound 4

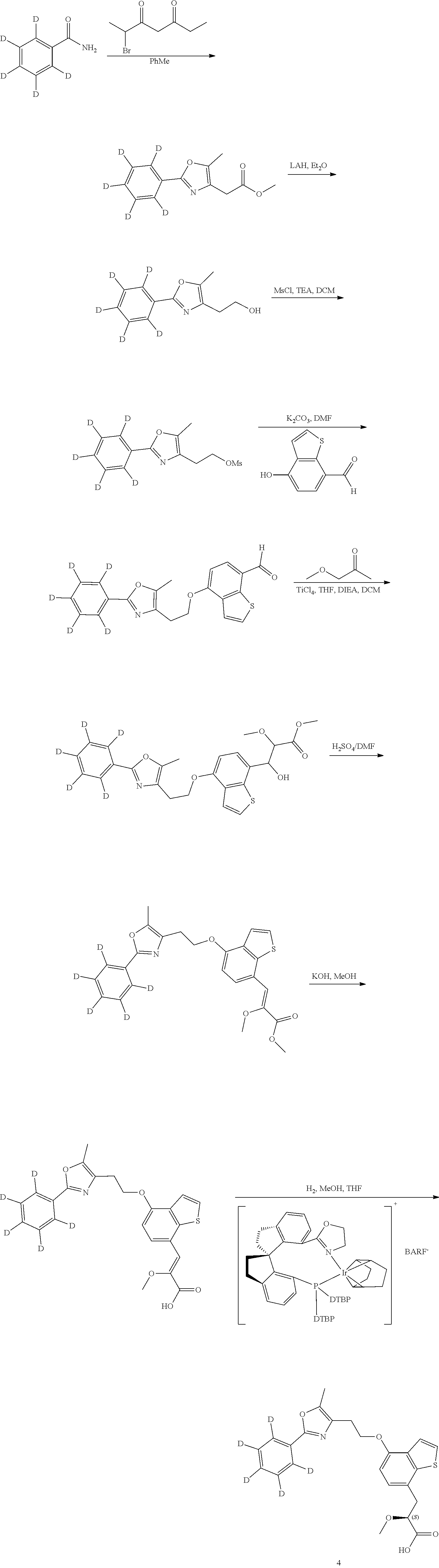

### Process Description

To a solution of benzamide-2,3,4,5,6-d₅ (9.6 g, 76.2 mmol, 1.0 eq) in toluene (150 mL) was added methyl 4-bromo-3-oxopentanoate (23.9 g, 114.3 mmol, 1.5 eq). After stirred at 110° C. for 10 hours, another batch of methyl 4-bromo-3-oxopentanoate (23.9 g, 114.3 mmol, 1.5 eq) was added and the mixture was continued to stir at 110° C. for another 20 hours. Then the reaction mixture was concentrated to give crude product which was purified by silica gel column (eluted with petroleum ether/EtOAc=15: 1) to afford methyl 2-(5-methyl-2-(phenyl-d₅)oxazol-4-yl)acetate (9.8 g, yield 54.4%) as a yellow oil.

¹HNMR (400 MHZ, CDCl₃) δ: 3.73 (s, 3H), 3.57 (s, 2H), 2.36 (s, 3H).

LC-MS (ESI⁺): 237.2 ([M+H]⁺).

To an ice-cooled solution of lithium aluminum hydride (2.4 g, 62.2 mmol, 1.5 eq) in Et₂O (100 mL) was added a solution of methyl 2-(5-methyl-2-(phenyl-d₅)oxazol-4-yl)acetate (9.8 g, 41.5 mmol, 1.0 eq) in Et₂O (100 mL) dropwise. Then the reaction mixture was allowed to warm to room temperature and stirred for 15 min. The reaction mixture was quenched with water (2.4 mL) and aq NaOH (15%, 2.4 mL) at 0° C. Then water (7.2 mL) was added to the reaction mixture and stirred at room temperature for 15 min. Na₂SO₄ (12 g) was added to the mixture. The mixture was filtered and concentrated to afford 2-(5-methyl-2-(phenyl-d₅)oxazol-4-yl)ethan-1-ol (7.0 g, yield 81.0%) as a white solid.

¹HNMR (400 MHZ, CDCl₃) δ: 3.93 (t, J=5.6 Hz, 2H), 2.72 (t, J=5.6 Hz, 2H), 2.34 (s, 3H).

LC-MS (ESI⁺): 209.0 ([M+H]⁺).

To a solution of 2-(5-methyl-2-(phenyl-d₅)oxazol-4-yl)ethan-1-ol (4.4 g, 21.3 mmol, 1.0 eq) in dichloromethane (45 mL) was added triethylamine (4.3 g, 42.6 mmol, 2.0 eq) at room temperature. The mixture was cooled to 0° C. and methanesulfonyl chloride (3.7 g, 32.0 mmol, 1.5 eq) was added dropwise over 10 min. The reaction was maintained at 5° C. for 2 hours and then quenched with 1 N HCl (10 mL), and extracted with dichloromethane (20 mL×2). The combined organic layer was washed with sat. aq NaHCO₃ (20 mL×2), brine (20 mL×2), dried over Na₂SO₄, filtered and concentrated under vacuum to afford 2-(5-methyl-2-(phenyl-d₅)oxazol-4-yl)ethyl methanesulfonate (6.1 g, crude) as a yellow solid, which was directly used to the next step without further purification.

¹HNMR (400 MHZ, CDCl₃) δ: 4.52 (t, J=6.6 Hz, 2H), 2.94 (t, J=6.6 Hz, 5H), 2.36 (s, 3H).

LC-MS (ESI⁺): 287.2 ([M+H]⁺).

Under argon, to a solution of 4-hydroxybenzo[b]thiophene-7-carbaldehyde (3.8 g, 21.3 mmol, 1.0 eq) in N,N-dimethylformamide (40 mL) was added K₂CO₃ (3.5 g, 25.6 mmol, 1.2 eq) at room temperature. The reaction mixture was heated to 86° C. and then a solution of 2-(5-methyl-2-(phenyl-d₅)oxazol-4-yl)ethyl methanesulfonate (6.1 g, 21.3 mmol, 1.0 eq) in DMF (20 mL) was added. The reaction mixture was stirred at 86° C. for 3 hours and then cooled and poured into water (150 mL), extracted with EtOAc (150 mL×2), and washed with water (100 mL×2) and brine (100 mL×2). The organic layer was dried over Na₂SO₄, filtered and concentrated to give a crude product, which was washed with EtOAc to afford 4-(2-(5-methyl-2-(phenyl-d₅)oxazol-4-yl)ethoxy)benzo[b]thiophene-7-carbaldehyde (5.3 g, yield 67.9%) as a yellow solid. The crude product was used for next step without further purification.

¹HNMR (400 MHZ, CDCl₃) δ: 10.05 (s, 1H), 7.80 (d, J=8.0 Hz, 1H), 7.53 (s, 2H), 6.94 (d, J=8.0 Hz, 1H), 4.53 (t, J=6.0 Hz, 2H), 3.12 (t, J=6.6 Hz, 2H), 2.42 (s, 3H).

LC-MS (ESI⁺): 368.9 ([M+H]⁺).

Under argon, to a solution of methyl 2-methoxyacetate (7.8 g, 74.9 mmol, 5.2 eq) in tetrahydrofuran (100 mL) was added TiCl₄ (14.2 g, 74.9 mmol, 5.2 eq) dropwise at 0° C. The yellow solution was stirred for 15 min, diisopropyl ethylamine (10.4 g, 80.6 mmol, 5.6 eq) was added. The solution was stirred for 15 min, a solution of 4-(2-(5-methyl-2-(phenyl-d₅)oxazol-4-yl)ethoxy)benzo[b]thiophene-7-carbaldehyde (5.3 g, 14.4 mmol, 1.0 eq) in DCM (100 mL) was added dropwise. After stirred for 60 min the reaction mixture was allowed to warm up to 20° C. and stirred overnight. The reaction mixture was cooled to 0° C. and quenched with ice water (150 mL). The organic layer was separated, the aqueous layer was extracted with dichloromethane (50 mL×2). The combined organic layer was washed with water (50 mL×2), dried over Na₂SO₄, filtered and evaporated to dryness to give methyl 3-hydroxy-2-methoxy-3-(4-(2-(5-methyl-2-(phenyl-d₅)oxazol-4-yl)ethoxy)benzo[b]thiophen-7-yl)propanoate (7.0 g, crude) as a red oil, which was directly used to the next step without further purification.

LC-MS (ESI⁺): 472.9([M+H]⁺).

To a solution of methyl 3-hydroxy-2-methoxy-3-(4-(2-(5-methyl-2-(phenyl-d₅)oxazol-4-yl)ethoxy)benzo[b]thiophen-7-yl)propanoate (crude, 6.5 g) in dimethylformamide (120 mL) was added con. H₂SO₄ (30 mL). The resulting dark brown solution was stirred at 100° C. overnight. The reaction solution was cooled to room temperature and poured into ice water (100 mL). The mixture was extracted with EtOAc (100 ml×2), the combine organic layer was washed with water (100 mL×2) and brine (100 mL×2). The organic layer was concentrated to afford a crude product, which was purified by silica gel column (eluted with petroleum ether/EtOAc=3:1) to afford methyl (Z)-2-methoxy-3-(4-(2-(5-methyl-2-(phenyl-d₅)oxazol-4-yl)ethoxy)benzo[b]thiophen-7-yl)acrylate (1.9 g, yield 30.6% for two steps) as an oil.

¹HNMR (400 MHZ, CDCl₃) δ: 8.10 (d, J=8.4 Hz, 1H), 7.48 (d, J=5.6 Hz, 1H), 7.34 (d, J=5.6 Hz, 1H), 7.21 (s, 1H), 6.86 (d, J=8.4 Hz, 1H), 4.47 (t, J=6.6 Hz, 2H), 3.88 (s, 3H), 3.77 (s, 3H), 3.10 (t, J=6.4 Hz, 2H), 2.41 (s, 3H).

LC-MS (ESI⁺): 455.1([M+H]⁺).

To a solution of methyl (Z)-2-methoxy-3-(4-(2-(5-methyl-2-(phenyl-d₅)oxazol-4-yl)ethoxy)benzo[b]thiophen-7-yl)acrylate (1.75 g, 3.9 mmol, 1.0 eq) in methanol (42 mL) and tetrahydrofuran (14 mL) was added a solution of KOH (1.3 g, 23.4 mmol, 6.0 eq) in water (4.2 mL). The reaction mixture was stirred at 65° C. for 2 hours. Then the reaction mixture was diluted with water (50 mL), concentrated and adjusted to pH=3 with 1N HCl. The mixture was extracted with dichloromethane/methanol=10:1 (150 mL). The organic layer was dried over Na₂SO₄, filtered and concentrated to afford (Z)-2-methoxy-3-(4-(2-(5-methyl-2-(phenyl-d₅)oxazol-4-yl)ethoxy)benzo[b]thiophen-7-yl)acrylic acid (1.1 g, yield 72.8%) as a white solid.

¹HNMR (400 MHz, CDCl₃) δ: 8.11 (d, J=8.4 Hz, 1H), 7.49 (d, J=5.2 Hz, 1H), 7.38-7.34 (m, 2H), 6.88 (d, J=8.4 Hz, 1H), 4.47 (t, J=8.0 Hz, 2H), 3.79 (s, 3H), 3.12 (t, J=6.6 Hz, 2H), 2.42 (s, 3H).

LC-MS (ESI⁺): 440.9 ([M+H]⁺).

A 30 mL stainless steel autoclave was charged with (Z)-2-methoxy-3-(4-(2-(5-methyl-2-(phenyl-d₅)oxazol-4-yl)ethoxy)benzo[b]thiophen-7-yl)acrylic acid (350.0 mg, 0.79 mmol, 1.0 eq), (S)-phenylethylamine (19.3 mg, 0.16 mmol, 0.2 eq), methanol (3.6 mL), tetrahydrofuran (2.4 mL) and Ir-cat ([((S)-DTBSIPHOX)Ir(COD)]BArF, 2.8 mg, 0.002 eq). The autoclave was sealed and the hydrogenation was stirred at 70° C. under 30 bar of hydrogen for 36 hours. The reaction solution was evaporated to dryness. The crude product was dissolved in DCM (20 mL) and washed with 1N HCl (10 mL). The organic layer was dried over Na₂SO₄, filtered and concentrated to afford a crude product, which was purified by Prep-HPLC to give compound 4 (205 mg, yield 58.6%) as a white solid.

¹HNMR (400 MHZ, CDCl₃) δ: 7.48 (d, J=5.6 Hz, 1H), 7.32 (d, J=5.6 Hz, 1H), 7.15 (d, J=8.0 Hz, 1H), 3.34 (t, J=6.4 Hz, 2H), 4.19 (dd, J=7.6, 4.8 Hz, 1H), 3.36-3.32 (m, 4H), 3.24-3.18 (m, 1H), 3.06 (t, J=6.4 Hz, 2H), 2.40 (s, 3H).

LC-MS (ESI⁺): 443.1 ([M+H]⁺).

Chiral HPLC (Chiralpak AD-3 4.6 mm*250 mm 3 μm, 90% hexanes/9.99% EtOH/0.01% TFA, 210 nm): 99.3% ee.

### Example 5: Synthesis of Compound 5

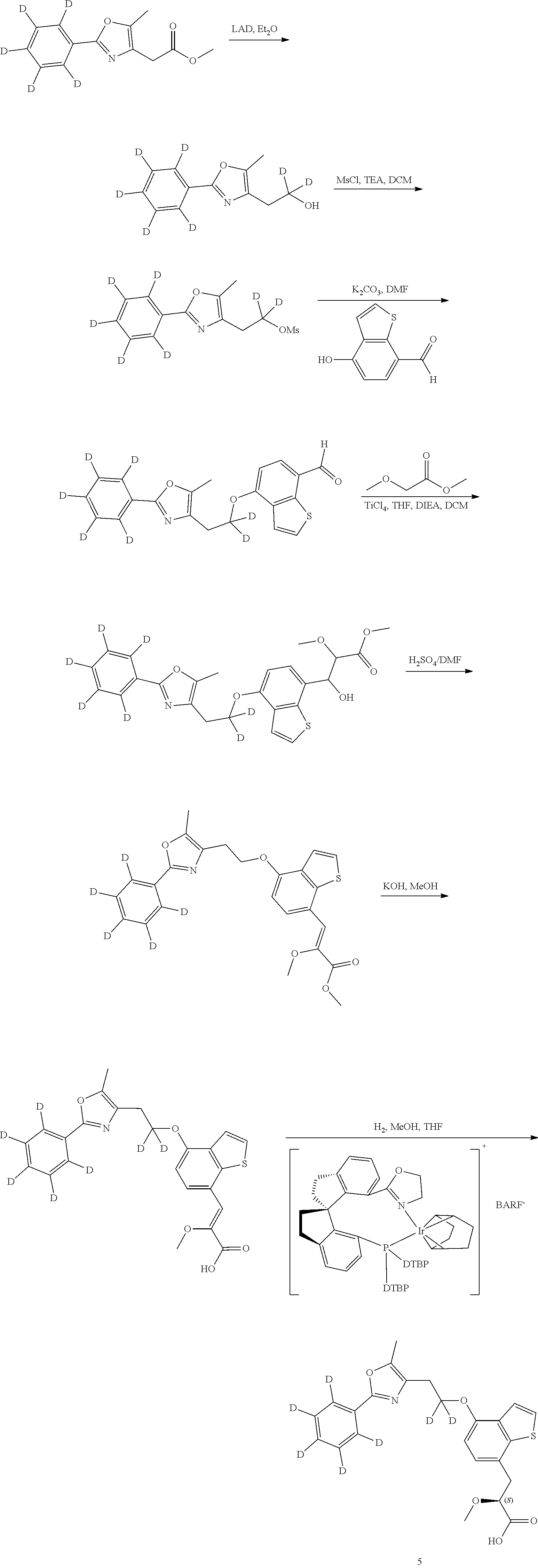

### Process Description

To an ice-cooled solution of LiAID₄ (1.0 g, 24.1 mmol, 1.5 eq) in diethyl ether (25 mL) was added a solution of methyl 2-(5-methyl-2-(phenyl-d₅)oxazol-4-yl)acetate (3.8 g, 16.1 mmol, 1.0 eq) in diethyl ether (15 mL) dropwise and stirred at room temperature for 15 min. The reaction mixture was quenched with water (1.0 mL) and aq. NaOH (15%, 1.0 mL) at 0° C. Then water (3.0 mL) was added. The mixture was stirred at room temperature for 15 min. Na₂SO₄ was added and the mixture was filtered and concentrated to afford 2-(5-methyl-2-(phenyl-d₅)oxazol-4-yl)ethan-1,1-d₂-1-ol (2.6 g, yield 78.2%) as a white solid. ¹HNMR (400 MHZ, CDCl₃) δ: 2.71 (s, 2H), 2.33 (s, 3H).

LC-MS (ESI⁺): 211.0 ([M+H]⁺).

To a solution of 2-(5-methyl-2-(phenyl-d₅)oxazol-4-yl)ethan-1,1-d₂-1-ol (2.5 g, 11.9 mmol, 1.0 eq) in DCM (45 mL) was added TEA (2.4 g, 23.8 mmol, 2.0 eq) and MsCl (2.0 g, 17.9 mmol, 1.5 eq) dropwise at 0° C. The reaction mixture was stirred at room temperature for 1 hour and then quenched with water (20 mL) and extracted with DCM (50 mL). The organic layer was dried (Na₂SO₄), filtered and concentrated to give 2-(5-methyl-2-(phenyl-d₅)oxazol-4-yl)ethyl-1,1-d₂ methanesulfonate (3.3 g, crude) as a yellow solid, which was used directly to the next step without further purification.

LC-MS (ESI⁺): 288.9 ([M+H]⁺).

Under argon, to a solution of 4-hydroxybenzo[b]thiophene-7-carbaldehyde (1.7 g, 9.5 mmol, 0.9 eq) in DMF (30 mL) was added K₂CO₃ (1.7 g, 12.4 mmol, 1.2 eq) at room temperature. The reaction mixture was heated to 86° C. and then a solution of 2-(5-methyl-2-(phenyl-d₅)oxazol-4-yl)ethyl-1,1-d₂ methanesulfonate (3.0 g, 10.6 mmol, 1.0 eq) in DMF (20 mL) was added. The reaction mixture was stirred at 86° C. for 4 hours and then poured into water (150 mL), and extracted with EtOAc (150 mL×2). The combined organic layer was washed with water (100 mL×2) and brine (100 mL×2). The organic layer was dried over Na₂SO₄, filtered and concentrated. The residue was triturated with EtOAc to afford 4-(2-(5-methyl-2-(phenyl-d₅)oxazol-4-yl)ethoxy-1,1-d₂)benzo[b]thiophene-7-carbaldehyde (2.2 g, yield 56.0%) as a yellow solid, which was used for next step without further purification.

¹HNMR (400 MHZ, CDCl₃) δ: 10.06 (s, 1H), 7.81 (d, J=8.0 Hz, 1H), 7.53 (s, 1H), 6.94 (d, J=8.0 Hz, 1H), 3.11(s, 2H), 2.42 (s, 3H).

LC-MS (ESI⁺): 370.9 ([M+H]⁺).

To a solution of methyl 2-methoxyacetate (3.2 g, 30.9 mmol, 5.2 eq) in THF (22 mL) was added TiCl₄ (5.9 g, 30.9 mmol, 5.2 eq) dropwise at 0° C., then DIEA (4.3 g, 33.0 mmol, 5.6 eq) was added. After 15 min, a solution of 4-(2-(5-methyl-2-(phenyl-d₅)oxazol-4-yl)ethoxy-1,1-d₂)benzo[b]thiophene-7-carbaldehyde (2.0 g, 5.9 mmol, 1.0 eq) in DCM (22 mL) was added. The reaction mixture was stirred at 0° C. for 4 hours and then quenched with water (40 mL) at 0° C. and extracted with DCM (40 mL×2). The organic layer was dried over Na₂SO₄, filtered and concentrated to give a crude product which was purified by silica gel column (eluted with petroleum ether/EtOAc=3:1) to afford methyl 3-hydroxy-2-methoxy-3-(4-(2-(5-methyl-2-(phenyl-d₅)oxazol-4-yl)ethoxy-1,1-d₂)benzo[b]thiophen-7-yl)propanoate (1.7 g, yield 60.7%) as a yellow oil.

LC-MS (ESI⁺): 474.8([M+H]⁺).

To a solution of methyl 3-hydroxy-2-methoxy-3-(4-(2-(5-methyl-2-(phenyl-d₅)oxazol-4-yl)ethoxy-1,1-d₂)benzo[b]thiophen-7-yl)propanoate (1.3 g, 2.7 mmol, 1.0 eq) in DMF (15 mL) was added con. H₂SO₄ (274 mg, 2.7 mmol, 1.0 eq) dropwise at room temperature. The reaction mixture was stirred at 100° C. for 5 hours and then quenched with ice-water (45 mL) and extracted with DCM (60 mL×2). The organic layer was dried (Na₂SO₄), filtered and concentrated to give a crude residue, which was triturated with EtOAc to afford methyl (Z)-2-methoxy-3-(4-(2-(5-methyl-2-(phenyl-d₅)oxazol-4-yl)ethoxy-1,1-d₂)benzo[b]thiophen-7-yl)acrylate (680 mg, yield 47.2%) as a yellow solid.

¹HNMR (400 MHZ, CDCl₃) δ: 8.11 (d, J=8.4 Hz, 1H), 7.48 (d, J=5.6 Hz, 1H), 7.34 (d, J=5.6 Hz, 1H), 7.21 (s, 1H), 6.85 (d, J=8.4 Hz, 1H), 3.88 (s, 3H), 3.77 (s, 3H), 3.08 (s, 2H), 2.41 (s, 3H).

LC-MS (ESI⁺): 457.0([M+H]⁺).

To a solution of methyl (Z)-2-methoxy-3-(4-(2-(5-methyl-2-(phenyl-d₅)oxazol-4-yl)ethoxy-1,1-d₂)benzo[b]thiophen-7-yl)acrylate (680 mg, 1.5 mmol, 1.0 eq) in MeOH/THF=3:1 (20 mL) was added a solution of KOH (501 mg, 8.9 mmol, 6.0 eq) in water (1.2 mL). The reaction mixture was stirred at 65° C. for 1 hour and then diluted with water (20 mL), concentrated and adjusted to pH=3 with 1 N HCl. The mixture was extracted with DCM/MeOH=10:1 (50 mL×2). The organic layer was dried over Na₂SO₄, filtered and concentrated to afford (Z)-2-methoxy-3-(4-(2-(5-methyl-2-(phenyl-d₅)oxazol-4-yl)ethoxy-1,1-d₂)benzo[b]thiophen-7-yl)acrylic acid (600 mg, yield 91.0%) as a yellow solid.

¹HNMR (400 MHZ, CDCl₃) δ: 8.11 (d, J=8.4 Hz, 1H), 7.49 (d, J=5.2 Hz, 1H), 7.35 (d, J=6.0 Hz, 2H), 6.87 (d, J=8.4 Hz, 1H), 3.79 (s, 3H), 3.10 (s, 2H), 2.42 (s, 3H).

LC-MS (ESI⁺): 442.7([M+H]⁺).

A 30 mL stainless steel autoclave was charged with (Z)-2-methoxy-3-(4-(2-(5-methyl-2-(phenyl-d₅)oxazol-4-yl)ethoxy-1,1-d₂)benzo[b]thiophen-7-yl)acrylic acid (300.0 mg, 0.7 mmol, 1.0 eq), (S)-phenylethylamine (16.5 mg, 0.14 mmol, 0.2 eq), MeOH (3.6 mL), THF (2.4 mL) and Ir-cat ([((S)-DTBSIPHOX)Ir(COD)]BArF, 4.8 mg, 0.004 eq). The autoclave was sealed and the hydrogenation was stirred at 70° C. under 30 bar of hydrogen for 36 hours. The reaction solution was evaporated to dryness. The crude product was dissolved in DCM (20 mL) and washed with 1 N HCl (10 mL). The organic layer was dried over Na₂SO₄, filtered and concentrated. The crude product was dissolved in isopropyl acetate at reflux and filtered. The filtrate was cooled to room temperature whereby crystallization started. The formed crystals were filtered, dried to afford compound 5 (150 mg, yield 48.2%) as a white solid.

¹HNMR (400 MHZ, CDCl₃) δ: 7.46 (d, J=5.2 Hz, 1H), 7.31 (d, J=5.2 Hz, 1H), 7.15 (d, J=8.0 Hz, 1H), 6.72 (d, J=8.0 Hz, 1H), 4.21-4.17 (m, 1H), 3.36-3.31 (m, 4H), 3.23-3.18 (m, 1H), 3.07 (s, 2H), 2.41 (s, 3H).

LC-MS (ESI⁺): 445.1 ([M+H]⁺).

Chiral HPLC (Chiralpak AD-3 4.6 mm*250 mm 3 μm, 90% hexanes/9.99% EtOH/0.01% TFA, 210 nm): 99.46% ee.

### Example 6: Synthesis of Compound 6

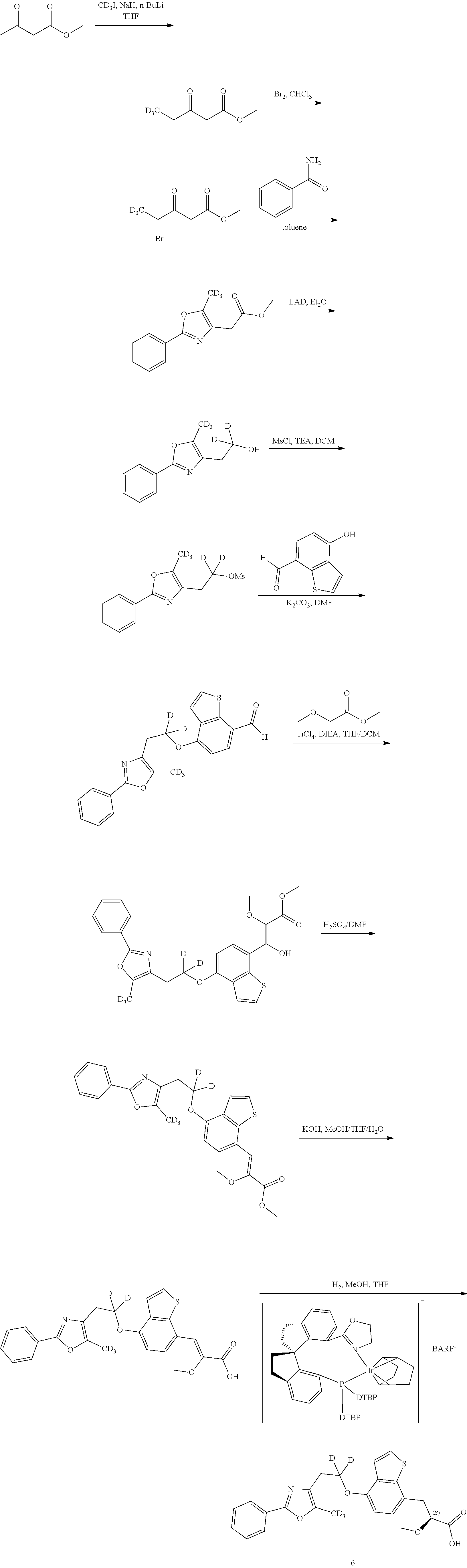

### Process Description

To a solution of NaH (60%, 28.8 g, 717.6 mmol, 1.3 eq) in THF (1.0 L) was added methyl 3-oxobutanoate (64.2 g, 552.0 mmol, 1.0 eq) at 0° C. under argon and stirred for 10 min. Then n-BuLi (2.4M, 300 mL, 717.6 mmol, 1.3 eq) was added dropwise at −20° C. and stirred for 5 min. CD₃I (100.0 g, 690.0 mmol, 1.25 eq) was added dropwise. The reaction mixture was stirred at room temperature for 4 hours and then quenched with sat. NH₄Cl (500 mL) and extracted with EtOAc (1000 mL×2). The organic layer was dried over Na₂SO₄, filtered and concentrated to afford methyl 3-oxopentanoate-5,5,5-d₃ (80.0 g, crude) as a yellow oil. ¹HNMR (400 MHz, CDCl₃) δ: 3.72 (s, 3H), 3.45 (s, 2H), 2.54 (s, 2H).

To a solution of methyl 3-oxopentanoate-5,5,5-d₃ (73.5 g, 552.0 mmol, 1.0 eq) in CHCl₃ (500.0 mL) was added a solution of Br₂ (101.1 g, 635.0 mmol, 1.15 eq) in CHCl₃ (200 mL) dropwise over a period of 30 min at 0° C. The reaction mixture was stirred at room temperature for 2 hours and then quenched with sat. aq NaHCO₃ (300 mL) and extracted with DCM (500 mL). The organic layer was dried over Na₂SO₄, filtered and concentrated to give a crude product which was purified by silica gel column (eluted with petroleum ether/EtOAc=100:1) to afford 4-bromo-3-oxopentanoate-5,5,5-d₃ (57.0 g, yield 48.7%) as a yellow oil. ¹HNMR (400 MHZ, CDCl₃) δ: 4.60 (s, 1H), 3.88-3.65 (m, 5H).

To a solution of benzamide (4.0 g, 32.7 mmol, 1.0 eq) in toluene (75 mL) was added methyl 4-bromo-3-oxopentanoate-5,5,5-d₃ (10.4 g, 49.0 mmol, 1.5 eq) at room temperature. The mixture was continued to stir at 110° C. for 12 hours, and then concentrated to give a crude product which was purified by silica gel column (eluted with petroleum ether/EtOAc=20:1) to afford methyl 2-(5-(methyl-d₃)-2-phenyloxazol-4-yl)acetate (3.6 g, yield 30.1%) as a yellow oil.

¹HNMR (400 MHZ, CDCl₃) δ: 7.99-7.97 (m, 2H), 7.44-7.40 (m, 3H), 3.73 (s, 3H), 3.58 (s, 2H)

LC-MS (ESI⁺): 235.0 ([M+H]⁺)

To a solution of methyl 2-(5-(methyl-d₃)-2-phenyloxazol-4-yl)acetate (3.6 g, 15.4 mmol, 1.0 eq) in Et₂O (36 mL) was added LiAID₄ (968 mg, 23 mol, 1.5 eq) portionwise at 0° C. The mixture was stirred at 5° C. for 2 hours, and then diluted with Et₂O (50 mL). The mixture was quenched with water (1 mL) and NaOH (0.15 g) in water (1 mL) at 0° C. Then water (3 mL) was added and stirred at room temperature for 15 min. After the addition of Na₂SO₄, the mixture was filtered and concentrated to afford a crude product which was purified by silica gel column (eluted with petroleum ether/EtOAc=3:1) to afford 2-(5-(methyl-d₃)-2-phenyloxazol-4-yl)ethan-1,1-d₂-1-ol (2.2 g, yield 68.6%) as a yellow oil.

¹HNMR (400 MHZ, CDCl₃) δ: 7.99-7.97 (m, 2H), 7.46-7.41 (m, 3H), 2.72 (s, 2H).

LC-MS (ESI⁺): 209.0 ([M+H]⁺).

To a solution of 2-(5-(methyl-d₃)-2-phenyloxazol-4-yl)ethan-1,1-d₂-1-ol (2.2 g, 10.6 mmol, 1.0 eq) in DCM (35 mL) was added TEA (2.1 g, 21.1 mmol, 2.0 eq). The mixture was cooled to 0° C., methanesulfonyl chloride (1.82 g, 15.8 mmol, 1.5 eq) was added dropwise. The reaction was stirred at 5° C. for 1 hour. The resulting reaction was adjusted to pH=7 with 1 N HCl, and extracted with DCM (50 mL×2). The combined organic layer was washed with sat. aq. NaHCO₃ (50 mL×2) and brine (50 mL×2), dried over Na₂SO₄, filtered and concentrated under vacuum to afford 2-(5-(methyl-d₃)-2-phenyloxazol-4-yl)ethyl-1,1-d₂ methanesulfonate (3.0 g, crude) as a yellow solid, which was directly used to the next step without further purification.

¹HNMR (400 MHZ, CDCl₃) δ: 7.98-7.95 (m, 2H), 7.46-7.41 (m, 3H), 3.13 (s, 3H), 2.94 (d, J=6.4 Hz, 2H).

LC-MS (ESI⁺): 287.0 ([M+H]⁺).

To a solution of 4-hydroxybenzo[b]thiophene-7-carbaldehyde (1.7 g, 9.6 mmol, 0.9 eq) in DMF (20 mL) was added K₂CO₃ (1.76 g, 12.7 mmol, 1.2 eq). The reaction mixture was heated to 86° C. under argon atmosphere and a solution of 2-(5-(methyl-d₃)-2-phenyloxazol-4-yl)ethyl-1,1-d₂ methanesulfonate (3.0 g, crude, 10.6 mmol, 1.0 eq) in DMF (10 mL) was added. The reaction mixture was stirred at 86° C. for 4 hours and then poured into water (50 mL), and extracted with EtOAc (50 mL×3). The combined organic layer was washed with water (50 mL×2) and brine (50 mL×2). The organic layer was dried over Na₂SO₄, filtered and concentrated to give a crude product, which was triturated with EtOAc and filtered to afford 4-(2-(5-(methyl-d₃)-2-phenyloxazol-4-yl)ethoxy-1,1-d₂)benzo[b]thiophene-7-carbaldehyde (2.4 g, yield 61.4% for two steps) as a yellow solid.

¹HNMR (400 MHZ, CDCl₃) δ: 10.06 (d, J=2.4 Hz, 1H), 7.99-7.97 (m, 2H), 7.81 (dd, J=8.0, 2.4 Hz, 1H), 7.53 (d, J=2.4, 2H), 7.43-7.39 (m, 3H), 6.94 (dd, J=8.0, 2.4 Hz, 1H)), 3.11 (s, 2H).

LC-MS (ESI⁺): 369.2 ([M+H]⁺).

To a solution of methyl 2-methoxyacetate (3.5 g, 33.9 mmol, 5.2 eq) in THF (24 mL) was added TiCl₄ (6.4 g, 33.9 mmol, 5.2 eq) dropwise at 0° C. under argon. After stirring the yellow solution for 15 min, DIEA (4.7 g, 36.5 mmol, 5.6 eq) was added. The solution was stirred for 15 min. A solution of 4-(2-(5-(methyl-d₃)-2-phenyloxazol-4-yl)ethoxy-1,1-d₂)benzo[b]thiophene-7-carbaldehyde (2.4 g, 6.5 mmol, 1.0 eq) in DCM (24 mL) was added dropwise. The reaction mixture was allowed to warm up to 20° C. and stirred overnight. The reaction mixture was cooled to 0° C. and quenched with ice water (100 mL). The organic layer was separated, and the aqueous layer was extracted with DCM (50 mL×2). The combined organic layer was washed with water (50 mL×3), dried over Na₂SO₄, filtered and evaporated to dryness. The crude product was purified by silica gel column (eluted with petroleum ether/EtOAc=3:1) to afford methyl 3-hydroxy-2-methoxy-3-(4-(2-(5-(methyl-d₃)-2-phenyloxazol-4-yl)ethoxy-1,1-d₂)benzo[b]thiophen-7-yl)propanoate (1.9 g, yield 61.9%) as a yellow solid.

LC-MS (ESI⁺): 473.2 ([M+H]⁺).

To a solution of methyl 3-hydroxy-2-methoxy-3-(4-(2-(5-(methyl-d₃)-2-phenyloxazol-4-yl)ethoxy-1,1-d₂)benzo[b]thiophen-7-yl)propanoate (1.7 g, 3.6 mmol, 1.0 eq) in DMF (20 mL) was added con. H₂SO₄ (540 mg, 7.2 mmol, 2.0 eq) dropwise at room temperature. The reaction mixture was stirred at 100° C. for 16 hours and then quenched with ice-water (100 mL) and extracted with DCM (50 mL×3). The organic layer was dried over Na₂SO₄, filtered and concentrated to give a crude product. The crude product was triturated with EtOAc and filtrated to afford methyl (Z)-2-methoxy-3-(4-(2-(5-(methyl-d₃)-2-phenyloxazol-4-yl)ethoxy-1,1-d₂)benzo[b]thiophen-7-yl)acrylate (1.0 g, yield 61.1%) as an off-white solid.

¹HNMR (400 MHZ, CDCl₃) δ: 8.10 (d, J=8.4 Hz, 1H), 8.00-7.98 (m, 2H), 7.48 (d, J=5.6 Hz, 1H), 7.44-7.42 (m, 3H), 7.34 (d, J=5.2 Hz, 1H), 7.21 (s, 1H), 6.85 (d, J=8.4 Hz, 1H), 3.88 (s, 3H), 3.77 (s, 3H), 3.08 (s, 2H).

LC-MS (ESI⁺): 455.2 ([M+H]⁺).

To a solution of methyl (Z)-2-methoxy-3-(4-(2-(5-(methyl-d₃)-2-phenyloxazol-4-yl)ethoxy-1,1-d₂)benzo[b]thiophen-7-yl)acrylate (1.0 g, 2.4 mmol, 1.0 eq) in MeOH/THF (3:1, 32 mL) was added a solution of KOH (792 mg, 14.1 mmol, 6.0 eq) in water (2 mL). The reaction mixture was stirred at 65° C. for 1.5 hours and then diluted with water (50 mL), concentrated and adjusted to pH=3 with 1 N HCl. The mixture was extracted with DCM/MeOH (10:1, 50 mL×2). The organic layer was dried over Na₂SO₄, filtered and concentrated to afford (Z)-2-methoxy-3-(4-(2-(5-(methyl-d₃)-2-phenyloxazol-4-yl)ethoxy-1,1-d₂)benzo[b]thiophen-7-yl)acrylic acid (770 mg, yield 72.8%) as an off-white solid.

¹HNMR (400 MHZ, DMSO-d₆) δ: 8.12 (d, J=8.4 Hz, 1H), 8.01-7.98 (m, 2H), 7.49 (d, J=5.6 Hz, 1H), 7.46-7.41 (m, 3H), 7.36-7.34 (m, 2H), 6.87 (d, J=8.4 Hz, 1H), 3.79 (s, 3H), 3.10 (s, 2H).

LC-MS (ESI⁺): 441.2 ([M+H]⁺).

A 30 mL stainless steel autoclave was charged with (Z)-2-methoxy-3-(4-(2-(5-(methyl-d₃)-2-phenyloxazol-4-yl)ethoxy-1,1-d₂)benzo[b]thiophen-7-yl)acrylic acid (300.0 mg, 0.7 mmol, 1.0 eq), (S)-phenylethylamine (16.5 mg, 0.14 mmol, 0.2 eq), MeOH (3.6 mL), THF (2.4 mL) and Ir-cat ([((S)-DTBSIPHOX)Ir(COD)]BArF, 4.8 mg, 0.004 eq). The autoclave was sealed and the hydrogenation was stirred at 70° C. under 30 bar of hydrogen for 36 hours. The reaction solution was evaporated to dryness. The crude product was dissolved in DCM (20 mL) and washed with 1N HCl (10 mL). The organic layer was dried over Na₂SO₄, filtered and concentrated. The crude product was dissolved in isopropyl acetate at reflux and filtered. The filtrate was cooled to room temperature whereby crystallization started. The formed crystals were filtered, dried to afford compound 6 (134 mg, yield 44.5%) as a white solid.

¹HNMR (400 MHZ, CDCl₃) δ: 7.97 (dd, J=8.0, 2.8 Hz, 1H), 7.48-7.40 (m, 4H), 7.31 (d, J=5.6 Hz, 1H), 7.15 (d, J=8.0 Hz, 1H), 6.72 (d, J=8.0 Hz, 1H), 4.21-4.18 (m, 1H), 3.33-3.31 (m, 4H), 3.24-3.18 (m, 1H), 3.05 (s, 2H).

LC-MS (ESI⁺): 443.2 ([M+H]⁺).

Chiral HPLC (Chiralpak AD-3 4.6 mm*250 mm 3 μm, 90% hexanes/9.99% EtOH/0.01% TFA, 210 nm): 99.51% ee.

### Example 7: Synthesis of Compound 7

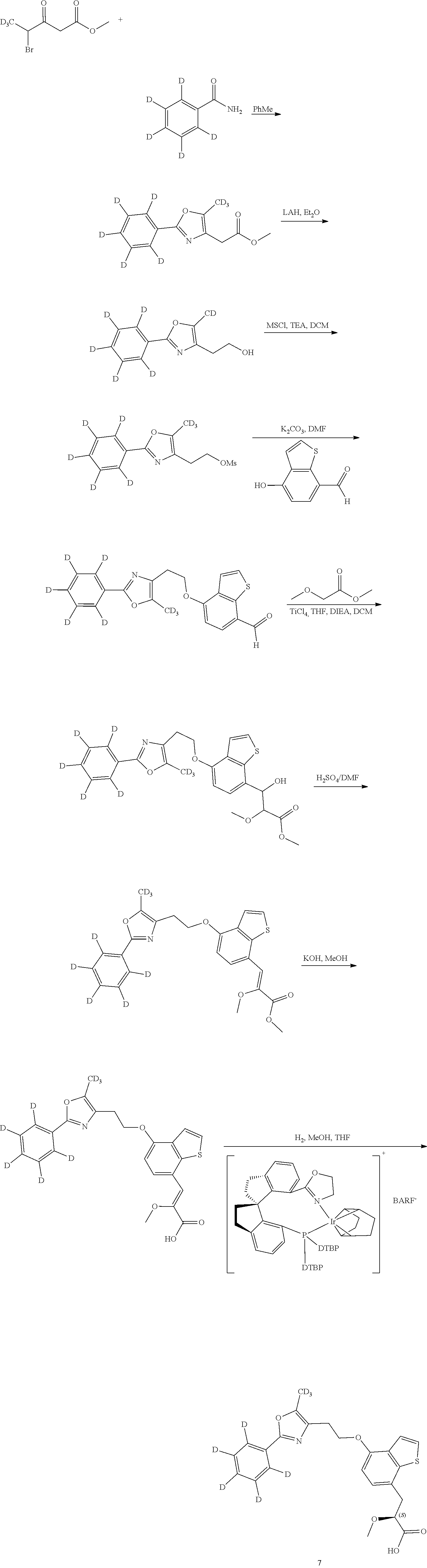

### Process Description

To a solution of ((4-bromo-3-oxopentanoyl-5,5,5-d₃)oxy)methylium (9.0 g, 70.8 mmol, 1.0 eq) in toluene (90.0 mL) was added benzamide-2,3,4,5,6-d₅ (44.8 g, 212.3 mmol, 3.0 eq) in two batches over a period of 10 hours. The mixture was continued to stir at 110° C. for 20 hours. Then the reaction mixture was concentrated to give a crude product which was purified by silica gel column (eluted with petroleum ether/EtOAc=15:1) to afford methyl 2-(5-(methyl-d₃)-2-(phenyl-d₅)oxazol-4-yl)acetate (7.3 g, yield 43.2%) as a yellow oil.

¹HNMR (400 MHz, CDCl₃) δ: 3.72 (s, 3H), 3.58 (s, 2H).

LC-MS (ESI⁺): 240.2 ([M+H]⁺).

To an ice-cooled solution of LAH (832.8 mg, 21.9 mmol, 1.5 eq) in diethyl ether (25 mL) was added a solution of methyl 2-(5-(methyl-d₃)-2-(phenyl-d₅)oxazol-4-yl)acetate (3.5 g, 14.6 mmol, 1.0 eq) in diethyl ether (25 mL) dropwise and stirred at room temperature for 15 min. The reaction mixture was quenched with water (1.0 mL) and aq. NaOH (15%, 1.0 mL) at 0° C. Water (3.0 mL) was added and stirred at room temperature for 15 min. After the addition of Na₂SO₄ the mixture was filtered and concentrated to afford 2-(5-(methyl-d₃)-2-(phenyl-d₅)oxazol-4-yl)ethan-1-ol (2.8 g, yield 87.7%) as a white solid.

¹HNMR (400 MHZ, CDCl₃) δ: 3.91 (t, J=6.0 Hz, 2H), 2.71 (t, J=6.0 Hz, 2H), 3.35 (brs, 1H).

LC-MS (ESI⁺): 212.1 ([M+H]⁺).

To a solution of 2-(5-(methyl-d₃)-2-(phenyl-d₅)oxazol-4-yl)ethan-1-ol (2.8 g, 13.4 mmol, 1.0 eq) in DCM (45 mL) was added TEA (2.7 g, 26.7 mmol, 2.0 eq) and methanesulfonyl chloride (2.3 g, 20.0 mmol, 1.5 eq) dropwise at 0° C. The reaction mixture was stirred at room temperature for 1 hour and then quenched with water (20 mL) and extracted with DCM (50 mL×2). The organic layer was dried over Na₂SO₄, filtered and concentrated to give 2-(5-(methyl-d₃)-2-(phenyl-d₅)oxazol-4-yl)ethyl methanesulfonate (3.8 g, crude) as a yellow solid, which was directly used to the next step without further purification.

LC-MS (ESI⁺): 290.1 ([M+H]⁺).

Under argon, to a solution of 4-hydroxybenzo[b]thiophene-7-carbaldehyde (2.1 g, 13.1 mmol, 0.9 eq) in DMF (30 mL) was added K₂CO₃ (2.17 g, 15.7 mmol, 1.2 eq) at room temperature. The reaction mixture was heated to 85° C. and then a solution of 2-(5-(methyl-d₃)-2-(phenyl-d₅)oxazol-4-yl)ethyl methanesulfonate (3.8 g, 13.1 mmol, 1.0 eq) in DMF (20 mL) was added. The reaction mixture was stirred at 85° C. for 4 hours and then poured into water (150 mL), and extracted with EtOAc (150 mL×2). The combined organic layer was washed with water (100 mL×2) and brine (100 mL×2). The organic layer was dried over Na₂SO₄, filtered and concentrated to give a crude product which was triturated with EtOAc to afford 4-(2-(5-(methyl-d₃)-2-(phenyl-d₅)oxazol-4-yl)ethoxy)benzo[b]thiophene-7-carbaldehyde (2.7 g, yield 55.5%) as an off-white solid.

¹HNMR (400 MHZ, CDCl₃) δ: 10.06 (s, 1H), 7.81 (d, J=8.0 Hz, 1H), 7.53 (s, 1H), 6.94 (d, J=8.0 Hz, 1H), 4.53 (t, J=6.4 Hz, 2H), 2.71 (t, J=6.4 Hz, 2H).

LC-MS (ESI⁺): 372.1 ([M+H]⁺).

To a solution of methyl 2-methoxyacetate (3.9 g, 37.8 mmol, 5.2 eq) in THF (30 mL) was added TiCl₄ (7.2 g, 37.8 mmol, 5.2 eq) and DIEA (5.3 g, 40.7 mmol, 5.6 eq) dropwise at 0° C. After 15 min, a solution of 4-(2-(5-(methyl-d₃)-2-(phenyl-d₅)oxazol-4-yl)ethoxy)benzo[b]thiophene-7-carbaldehyde (2.7 g, 7.3 mmol, 1.0 eq) in DCM (30 mL) was added. The reaction mixture was stirred at 0° C. for 4 hours and then quenched with water (40 mL) at 0° C. and extracted with DCM (40 mL×2). The organic layer was dried over Na₂SO₄, filtered and concentrated to give a crude product which was purified by silica gel column (eluted with petroleum ether/EtOAc=3:1) to afford methyl 3-hydroxy-2-methoxy-3-(4-(2-(5-(methyl-d₃)-2-(phenyl-d₅)oxazol-4-yl)ethoxy)benzo[b]thiophen-7-yl)propanoate (1.7 g, yield 49.2%) as a yellow oil.

LC-MS (ESI⁺): 476.1 ([M+H]⁺).

To a solution of methyl 3-hydroxy-2-methoxy-3-(4-(2-(5-(methyl-d₃)-2-(phenyl-d₅)oxazol-4-yl)ethoxy)benzo[b]thiophen-7-yl)propanoate (1.7 g, 3.6 mmol, 1.0 eq) in DMF (15 mL) was added con. H₂SO₄ (536 mg, 5.4 mmol, 1.5 eq) dropwise at room temperature. The reaction mixture was stirred at 100° C. for 5 hours and then quenched with ice-water (45 mL) and extracted with DCM (60 mL×2). The organic layer was dried over Na₂SO₄, filtered and concentrated to give a crude residue, which was triturated with EtOAc to afford methyl (Z)-2-methoxy-3-(4-(2-(5-(methyl-d₃)-2-(phenyl-d₅)oxazol-4-yl)ethoxy)benzo[b]thiophen-7-yl)acrylate (840 mg, yield 51.4%) as a yellow solid.

¹HNMR (400 MHZ, CDCl₃) δ: 8.10 (d, J=8.4 Hz, 1H), 7.48 (d, J=5.6 Hz, 1H), 7.34 (d, J=5.6 Hz, 1H), 7.21 (s, 1H), 6.86 (d, J=8.4 Hz, 1H), 4.46 (t, J=6.4 Hz, 2H), 3.88 (s, 3H), 3.77 (s, 3H), 3.10 (t, J=6.4 Hz, 2H).

LC-MS (ESI⁺): 458.0 ([M+H]⁺).

To a solution of methyl (Z)-2-methoxy-3-(4-(2-(5-(methyl-d₃)-2-(phenyl-d₅)oxazol-4-yl)ethoxy)benzo[b]thiophen-7-yl)acrylate (840.0 mg, 1.8 mmol, 1.0 eq) in MeOH/THF (3:1, 32 mL) was added a solution of KOH (618.0 mg, 11.0 mmol, 6.0 eq) in water (2.0 mL). The reaction mixture was stirred at 65° C. for 1 hour and then diluted with water (20 mL), concentrated and adjusted to pH=3 with 1N HCl. The mixture was extracted with DCM/MeOH (10:1, 50 mL×2). The organic layer was dried over Na₂SO₄, filtered and concentrated to afford (Z)-2-methoxy-3-(4-(2-(5-(methyl-d₃)-2-(phenyl-d₅)oxazol-4-yl)ethoxy)benzo[b]thiophen-7-yl)acrylic acid (700 mg, yield 85.7%) as an off-white solid.

¹HNMR (400 MHZ, CDCl₃) δ: 8.11 (d, J=8.4 Hz, 1H), 7.49 (d, J=5.6 Hz, 1H), 7.35 (d, J=5.2 Hz, 2H), 6.87 (d, J=8.8 Hz, 1H), 4.46 (t, J=6.4 Hz, 2H), 3.79 (s, 3H), 3.11 (t, J=6.4 Hz, 2H).

LC-MS (ESI⁺): 444.1([M+H]⁺).

A 30 mL stainless steel autoclave was charged with (Z)-2-methoxy-3-(4-(2-(5-(methyl-d₃)-2-(phenyl-d₅)oxazol-4-yl)ethoxy)benzo[b]thiophen-7-yl)acrylic acid (300.0 mg, 0.7 mmol, 1.0 eq), (S)-phenylethylamine (16.5 mg, 0.14 mmol, 0.2 eq), MeOH (3.6 mL), THF (2.4 mL) and Ir-cat ([((S)-DTBSIPHOX)Ir(COD)]BArF, 4.8 mg, 0.004 eq). The autoclave was sealed and the hydrogenation was stirred at 70° C. under 30 bar of hydrogen for 36 hours. The reaction solution was evaporated to dryness. The crude product was dissolved in DCM (20 mL) and washed with 1N HCl (10 mL). The organic layer was dried over Na₂SO₄, filtered and concentrated. The crude product was dissolved in isopropyl acetate at reflux and filtered. The filtrate was cooled to room temperature whereby crystallization started. The formed crystals were filtered, dried to afford compound 7 (130 mg, yield 41.7%) as a white solid.

¹HNMR (400 MHZ, CDCl₃) δ: 7.46 (d, J=5.2 Hz, 1H), 7.31 (d, J=5.2 Hz, 1H), 7.15 (d, J=8.0 Hz, 1H), 6.72 (d, J=8.0 Hz, 1H), 4.34 (t, J=6.4 Hz, 2H), 4.21-4.18 (m, 1H), 3.36-3.31 (m, 4H), 3.24-3.18 (m, 1H), 3.08 (t, J=6.4 Hz, 2H).

LC-MS (ESI⁺): 446.1 ([M+H]⁺).

Chiral HPLC (Chiralpak AD-3 4.6 mm*250 mm 3 μm, 90% hexanes/9.99% EtOH/0.01% TFA, 210 nm): 98.68% ee.

### Example 8: Synthesis of Compound 8

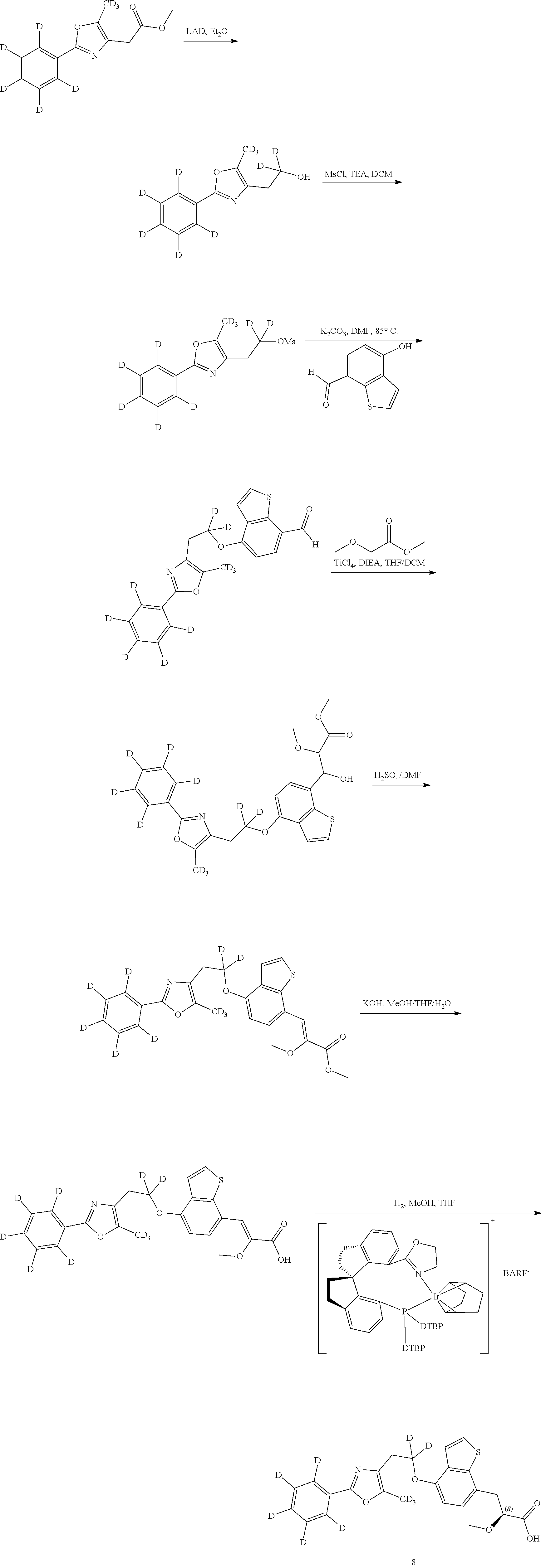

### Process Description

To a solution of methyl 2-(5-(methyl-d₃)-2-(phenyl-d₅)oxazol-4-yl)acetate (3.8 g, 15.9 mmol, 1.0 eq) in Et₂O (70 mL) was added LiAID₄ (976 mg, 23.8 mol, 1.5 eq) portion wise at 0° C. The mixture was stirred at 5° C. for 2 hours, and then diluted with Et₂O (50 mL). The reaction was quenched with water (1 mL) and a solution of NaOH (0.15 g) in water (1 mL) at 0° C. Water (3 mL) was added and the mixture was stirred for 15 min. After the addition of Na₂SO₄, the mixture was filtered and concentrated to afford a crude product which was purified by silica gel column (eluted with petroleum ether/EtOAc=3:1) to afford 2-(5-(methyl-d₃)-2-(phenyl-d₅)oxazol-4-yl)ethan-1,1-d₂-1-ol (2.9 g, yield 83.1%) as a yellow oil.

¹HNMR (400 MHZ, CDCl₃) δ: 3.19 (brs, 1H), 2.73 (s, 2H).

LC-MS (ESI⁺): 214.0 ([M+H]⁺).

To a solution of 2-(5-(methyl-d₃)-2-(phenyl-d₅)oxazol-4-yl)ethan-1,1-d₂-1-ol (2.9 g, 13.22 mmol, 1.0 eq) in DCM (45 mL) was added TEA (2.7 g, 26.5 mmol, 2.0 eq). The mixture was cooled to 0° C., and methanesulfonyl chloride (2.3 g, 19.8 mmol, 1.5 eq) was added dropwise. The reaction mixture was stirred at room temperature for 1 hour and then quenched with water (20 mL) and extracted with DCM (50 mL×2). The organic layer was dried over Na₂SO₄, filtered and concentrated to give 2-(5-(methyl-d₃)-2-(phenyl-d₅)oxazol-4-yl)ethyl-1,1-d₂ methanesulfonate (4.0 g, crude) as a yellow solid, which was directly used to the next step without further purification.

LC-MS (ESI⁺): 292.2 ([M+H]⁺).

To a solution of 4-hydroxybenzo[b]thiophene-7-carbaldehyde (2.12 g, 11.9 mmol, 0.9 eq) in DMF (30 mL) was added K₂CO₃ (2.19 g, 15.86 mmol, 1.2 eq). The reaction mixture was heated to 85° C. under argon atmosphere and a solution of 2-(5-(methyl-d₃)-2-(phenyl-d₅)oxazol-4-yl)ethyl-1,1-d₂ methanesulfonate (3.85 g, crude, 13.22 mmol, 1.0 eq) in DMF (10 mL) was added. The reaction mixture was stirred at 85° C. for 4 hours and then poured into water (50 mL), and extracted with EtOAc (50 mL×3). The combined organic layer was washed with water (50 mL×2) and brine (50 mL×2). The organic layer was dried over Na₂SO₄, filtered and concentrated to give a crude product, which was triturated with EtOAc to afford 4-(2-(5-(methyl-d₃)-2-(phenyl-d₅)oxazol-4-yl)ethoxy-1,1-d₂)benzo[b]thiophene-7-carbaldehyde (2.4 g, yield 50.2% for two steps) as an off-white solid.

¹HNMR (400 MHZ, CDCl₃) δ: 10.06 (s, 1H), 7.81 (d, J=8 Hz, 1H), 7.53 (s, 2H), 6.94 (d, J=8 Hz, 1H)), 3.11 (s, 2H).

LC-MS (ESI⁺): 374.2 ([M+H]⁺).

To a solution of methyl 2-methoxyacetate (3.48 g, 33.4 mmol, 5.2 eq) in THF (24 mL) was added TiCl₄ (6.34 g, 33.4 mmol, 5.2 eq) dropwise at 0° C. under argon. After stirring the yellow solution for 15 min, DIEA (4.65 g, 36 mmol, 5.6 eq) was added. The solution was stirred for 15 min. A solution of 4-(2-(5-(methyl-d₃)-2-(phenyl-d₅)oxazol-4-yl)ethoxy-1,1-d₂)benzo[b]thiophene-7-carbaldehyde (2.4 g, 6.4 mmol, 1.0 eq) in DCM (24 mL) was added dropwise and stirred for 60 min. The reaction mixture was allowed to warm up to 20° C. and stirred overnight. Then the reaction was cooled to 0° C. and quenched with ice water (100 mL). The organic layer was separated and the aqueous layer was extracted with DCM (50 mL×2). The combined organic layer was washed with water (50 mL×3), dried over Na₂SO₄, filtered and evaporated to dryness. The crude product was purified by silica gel column (eluted with petroleum ether/EtOAc=3:1) to afford methyl 3-hydroxy-2-methoxy-3-(4-(2-(5-(methyl-d₃)-2-(phenyl-d₅)oxazol-4-yl)ethoxy-1,1-d₂)benzo[b]thiophen-7-yl)propanoate (1.85 g, yield 60.9%) as a yellow solid.

LC-MS (ESI⁺): 478.2 ([M+H]⁺).

To a solution of methyl 3-hydroxy-2-methoxy-3-(4-(2-(5-(methyl-d₃)-2-(phenyl-d₅)oxazol-4-yl)ethoxy-1,1-d₂)benzo[b]thiophen-7-yl)propanoate (1.85 g, 3.9 mmol, 1.0 eq) in DMF (20 mL) was added concentrated H₂SO₄ (582 mg, 5.8 mmol, 1.5 eq) dropwise at room temperature. The reaction mixture was stirred at 100° C. for 16 hours and then quenched with ice-water (100 mL) and extracted with DCM (50 mL×3). The organic layer was dried (Na₂SO₄) and concentrated to give a crude residue, which was triturated with EtOAc and filtrated to afford methyl (Z)-2-methoxy-3-(4-(2-(5-(methyl-d₃)-2-(phenyl-d₅)oxazol-4-yl)ethoxy-1,1-d₂)benzo[b]thiophen-7-yl)acrylate (1.4 g, yield 78.2%) as a yellow solid.

¹HNMR (400 MHZ, CDCl₃) δ: 8.10 (d, J=8.4 Hz, 1H), 7.48 (d, J=5.6 Hz, 1H), 7.34 (d, J=5.6 Hz, 1H), 7.21 (s, 1H), 6.85 (d, J=8.4 Hz, 1H), 3.88 (s, 3H), 3.77 (s, 3H), 3.08 (s, 2H).

LC-MS (ESI⁺): 460.2 ([M+H]⁺).

To a solution of methyl (Z)-2-methoxy-3-(4-(2-(5-(methyl-d₃)-2-(phenyl-d₅)oxazol-4-yl)ethoxy-1,1-d₂)benzo[b]thiophen-7-yl)acrylate (1.4 g, 3.05 mmol, 1.0 eq) in MeOH/THF (3:1, 44 mL) was added a solution of KOH (1.03 g, 18.3 mmol, 6.0 eq) in water (2 mL). The reaction mixture was stirred at 65° C. for 1.5 hours and then diluted with water (50 mL), concentrated and adjusted to pH=3 with 1 N HCl. The mixture was extracted with DCM/MeOH (10:1, 50 mL×2). The organic layer was dried over Na₂SO₄, filtered and concentrated to afford (Z)-2-methoxy-3-(4-(2-(5-(methyl-d₃)-2-(phenyl-d₅)oxazol-4-yl)ethoxy-1,1-d₂)benzo[b]thiophen-7-yl)acrylic acid (1.1 g, yield 81.0%) as a yellow solid.

¹HNMR (400 MHZ, DMSO-d₆) δ: 8.15 (d, J=8.4 Hz, 1H), 7.51 (d, J=5.2 Hz, 1H), 7.39-7.36 (m, 2H), 6.90 (d, J=8.4 Hz, 1H), 3.82 (s, 3H), 3.13 (s, 2H).

LC-MS (ESI⁺): 446.0 ([M+H]⁺).

A 30 mL stainless steel autoclave was charged with (Z)-2-methoxy-3-(4-(2-(5-(methyl-d₃)-2-(phenyl-d₅)oxazol-4-yl)ethoxy-1,1-d₂)benzo[b]thiophen-7-yl)acrylic acid (300.0 mg, 0.7 mmol, 1.0 eq), (S)-phenylethylamine (16.5 mg, 0.14 mmol, 0.2 eq), MeOH (3.6 mL), THF (2.4 mL) and Ir-cat ([((S)-DTBSIPHOX)Ir(COD)]BArF, 4.8 mg, 0.004 eq). The autoclave was sealed and the hydrogenation was stirred at 70° C. under 30 bar of hydrogen for 36 hours. The reaction solution was evaporated to dryness. The crude product was dissolved in DCM (20 mL) and washed with 1 N HCl (10 mL). The organic layer was dried over Na₂SO₄, filtered and concentrated. The crude product was dissolved in isopropyl acetate at reflux and filtered. The filtrate was cooled to room temperature whereby crystallization started. The formed crystals were filtered, dried to afford compound 8 (145 mg, yield 54.1%) as a white solid.

¹HNMR (400 MHZ, CDCl₃) δ: 7.45 (d, J=5.2 Hz, 1H), 7.31 (d, J=5.2 Hz, 1H), 7.15 (d, J=8.0 Hz, 1H), 6.72 (d, J=8.0 Hz, 1H), 4.21-4.18 (m, 1H), 3.35-3.31 (m, 4H), 3.23-3.20 (m, 1H), 3.07 (s, 2H).

LC-MS (ESI⁺): 448.2 ([M+H]⁺).

Chiral HPLC (Chiralpak AD-3 4.6 mm*250 mm 3 μm, 90% hexanes/9.99% EtOH/0.01% TFA, 210 nm): 99.79% ee.

### Example 9: Synthesis of Compound 9

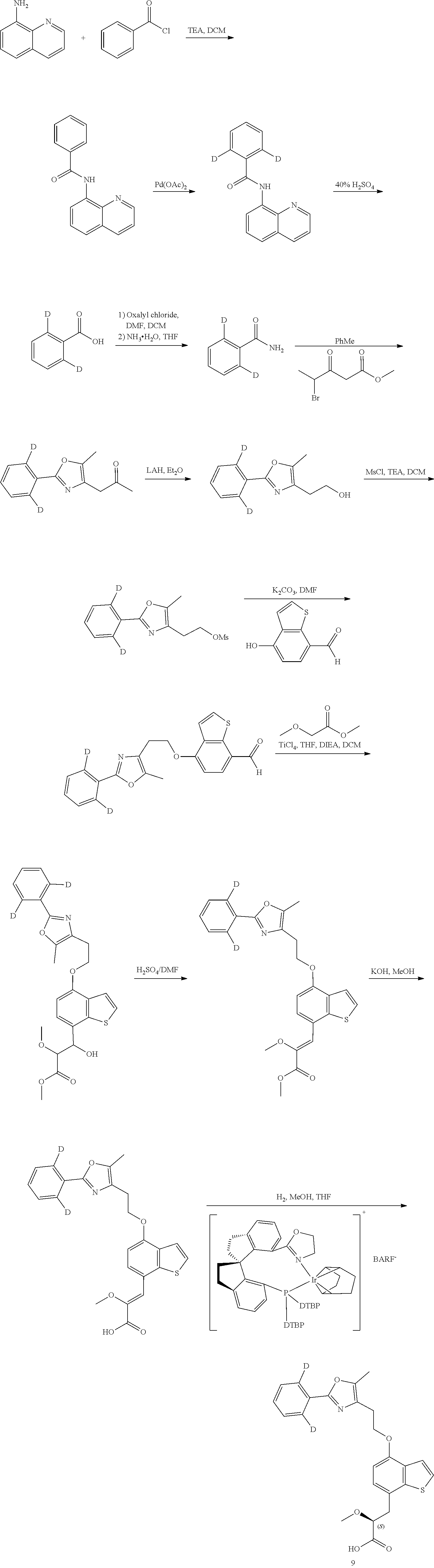

### Process Description

To a solution of quinolin-8-amine (30.0 g, 208.1 mmol, 1.0 eq) in DCM (300 mL) was added TEA (25.3 g, 249.7 mmol, 1.2 eq) at room temperature under argon. benzoyl chloride (35.1 g, 249.7 mmol, 1.2 eq) was added dropwise at 0° C. The reaction mixture was stirred at room temperature for 14 hours and then quenched with sat. NaHCO₃ and extracted with DCM (300 mL×2). The organic layer was dried over Na₂SO₄, filtered and concentrated to give a crude product which was purified by silica gel column (eluted with petroleum ether/EtOAc=15:1) to afford N-(quinolin-8-yl)benzamide (45.0 g, yield 87.1%) as a yellow solid.

¹HNMR (400 MHZ, CDCl₃) δ: 10.76 (s, 1H), 8.95 (dd, J=7.5 Hz, 1H), 8.86 (dd, J=4.2 Hz, 1H), 8.20 (dd, J=8.3 Hz, 1H), 8.11-8.08 (m, 2H), 7.63-7.53 (m, 5H), 7.49 (dd, J=8.3, 4.2 Hz, 1H).

LC-MS (ESI⁺): 280.1 ([M+Na]⁺).

A 300 mL stainless steel autoclave was charged with N-(quinolin-8-yl)benzamide (5.0 g, 20.1 mmol, 1.0 eq), Pd(OAc)₂ (904 mg, 4.1 mmol, 0.2 eq) and D₂O (100 mL). The autoclave was sealed and stirring at 140° C. for 36 hours. Then the reaction mixture was diluted with water (100 mL) and extracted with DCM (500 mL). The organic layer was dried over Na₂SO₄, filtered and concentrated to give crude product which was purified by silica gel column (eluted with petroleum ether/EtOAc=10:1) to afford N-(quinolin-8-yl)benzamide-2,6-d₂ (2.5 g, yield 49.6%) as a white solid.

¹HNMR (400 MHZ, CDCl₃) δ: 10.75 (s, 1H), 8.95 (dd, J=7.5 Hz, 1H), 8.86 (dd, J=4.2 Hz, 1H), 8.19 (dd, J=8.3 1H), 7.63-7.54 (m, 5H), 7.48 (dd, J=8.3 Hz, 1H).

LC-MS (ESI⁺): 250.1 ([M+H]⁺).

A mixture of N-(quinolin-8-yl)benzamide-2,6-d₂ (4.0 g, 16.0 mmol, 1.0 eq) and aq. H₂SO₄ (40%, 100 mL) was stirred at 120° C. for 14 hours. Then the reaction mixture was poured into water (150 mL) and extracted with EtOAc (200 mL). The organic layer was dried over Na₂SO₄, filtered and concentrated to afford benzoic-2,6-d₂ acid (1.8 g, yield 92.3%) as a white solid, which was used for next step without further purification (>98% D based HNMR).

¹HNMR (400 MHZ, CDCl₃) δ: 7.63 (t, J=7.4 Hz, 1H), 7.49 (d, J=7.6 Hz, 2H).

LC-MS (ESI⁺): 122.8 ([M−H]⁻).

To a solution of benzoic-2,6-d₂ acid (1.5 g, 12.1 mmol, 1.0 eq) in DCM (15 mL) was added Oxalyl chloride (2.3 g, 18.1 mmol, 1.5 eq) and DMF (few drops). The reaction mixture was stirred at room temperature for 1 hour and then concentrated and re-dissolved in THF (15 mL). NH₃·H₂O (25%, 15 mL) was added dropwise at 0° C. The resulting mixture was stirred at room temperature for 15 min and then extracted with DCM/MeOH=10:1 (200 mL×2). The organic layer was dried over Na₂SO₄, filtered and concentrated to afford benzamide-2,6-d₂ (1.2 g, yield 76.9%) as a white solid.

¹HNMR (400 MHZ, DMSO-d₆) δ: 7.93 (brs, 1H), 7.50-7.46 (m, 1H), 7.41 (d, J=6.9 Hz, 2H), 7.31 (brs, 1H).

LC-MS (ESI⁺): 124.2 ([M+H]⁺).

To a solution of benzamide-2,6-d₂ (1.2 g, 9.7 mmol, 1.0 eq) in toluene (30 mL) was added methyl 4-bromo-3-oxopentanoate (3.05 g, 14.7 mmol, 1.5 eq). After 10 hours, another batch of methyl 4-bromo-3-oxopentanoate (3.05 g, 14.7 mmol, 1.5 eq) was added. The mixture was stirred at 110° C. for 20 hours and then concentrated to a residue. The crude product was purified by silica gel column (eluted with petroleum ether/EtOAc=15:1) to afford methyl 2-(5-methyl-2-(phenyl-2,6-d₂)oxazol-4-yl)acetate (1.3 g, yield 57.5%) as a yellow oil.

¹HNMR (400 MHZ, CDCl₃) δ: 7.41 (t, J=7.6 Hz, 3H), 3.73 (s, 1H), 3.58 (s, 2H), 2.36 (s, 3H).

LC-MS (ESI⁺): 233.9 ([M+H]⁺).

To an ice-cooled solution of LiAH₄ (313.0 mg, 8.3 mmol, 1.5 eq) in diethyl ether (10 mL) was added a solution of methyl 2-(5-methyl-2-(phenyl-2,6-d₂)oxazol-4-yl)acetate (1.3 g, 5.5 mmol, 1.0 eq) in diethyl ether (10 mL) dropwise. The reaction mixture was stirred at room temperature for 15 min and then quenched with water (0.3 mL) and aq. NaOH (15%, 0.3 mL) at 0° C. Water (1.0 mL) was added and stirred at room temperature for 15 min. After the addition of Na₂SO₄, the mixture was filtered and concentrated to afford 2-(5-methyl-2-(phenyl-2,6-d₂)oxazol-4-yl)ethan-1-ol (1.0 g, yield 88.5%) as a white solid.

¹HNMR (400 MHZ, CDCl₃) δ: 7.43 (t, J=8.4 Hz, 3H), 3.93 (t, J=6.4 Hz, 2H), 2.73 (t, J=6.4 Hz, 2H), 2.34 (s, 3H).

LC-MS (ESI⁺): 206.2 ([M+H]⁺).

To a solution of 2-(5-methyl-2-(phenyl-2,6-d₂)oxazol-4-yl)ethan-1-ol (1.0 g, 4.9 mmol, 1.0 eq) in DCM (15 mL) was added TEA (986.0 mg, 9.7 mmol, 2.0 eq) and methanesulfonyl chloride (837 mg, 7.31 mmol, 1.5 eq) dropwise at 0° C. The reaction mixture was stirred at room temperature for 1 hour and then quenched with water (20 mL) and extracted with DCM (50 mL). The organic layer was dried (Na₂SO₄) and concentrated to give 2-(5-methyl-2-(phenyl-2,6-d₂)oxazol-4-yl)ethyl methanesulfonate (1.2 g, crude) as a yellow solid, which was used directly to the next step without further purification.

LC-MS (ESI⁺): 284.1 ([M+H]⁺).

Under argon, to a solution of 4-hydroxybenzo[b]thiophene-7-carbaldehyde (679.0 mg, 3.8 mmol, 0.9 eq) in DMF (10 mL) was added K₂CO₃ (697.0 mg, 5.0 mmol, 1.2 eq) at room temperature. The reaction mixture was heated to 86° C. and then a solution of 2-(5-methyl-2-(phenyl-2,6-d₂)oxazol-4-yl)ethyl methanesulfonate (1.2 g, 4.2 mmol, 1.0 eq) in DMF (10 mL) was added. The reaction mixture was stirred at 86° C. for 5 hours and then poured into water (100 mL) and extracted with EtOAc (150 mL×2). The combined organic layer was washed with water (100 mL×2) and brine (100 mL×2). The organic layer was dried over Na₂SO₄, filtered and concentrated. The crude product was triturated with EtOAc to afford 4-(2-(5-methyl-2-(phenyl-2,6-d₂)oxazol-4-yl)ethoxy)benzo[b]thiophene-7-carbaldehyde (960 mg, yield 62.7%) as a yellow solid, which was used for next step without further purification.

¹HNMR (400 MHZ, CDCl₃) δ: 10.06 (s, 1H), 7.81 (d, J=8.1 Hz, 1H), 7.53 (s, 2H), 7.43 (q, J=3.7 Hz, 3H), 6.95 (d, J=8.1 Hz, 1H), 4.54 (t, J=6.6 Hz, 2H), 3.13 (t, J=6.5 Hz, 2H), 2.42 (s, 3H).

LC-MS (ESI⁺): 365.9 ([M+H]⁺).

To a solution of methyl 2-methoxyacetate (1.4 g, 13.7 mmol, 5.2 eq) in THF (10 mL) was added TiCl₄ (2.6 g, 13.7 mmol, 5.2 eq) and DIEA (1.9 g, 14.7 mmol, 5.6 eq) was added dropwise at 0° C. After 15 min, a solution of 4-(2-(5-methyl-2-(phenyl-2,6-d₂)oxazol-4-yl)ethoxy)benzo[b]thiophene-7-carbaldehyde (960.0 mg, 2.63 mmol, 1.0 eq) in DCM (10 mL) was added. The reaction mixture was stirred at 0° C. for 4 hours and then quenched with water (40 mL) at 0° C. and extracted with DCM (40 mL×2). The organic layer was dried over Na₂SO₄, filtered and concentrated to give a crude product, which was purified by silica gel column (eluted with petroleum ether/EtOAc=3:1) to afford methyl 3-hydroxy-2-methoxy-3-(4-(2-(5-methyl-2-(phenyl-2,6-d₂)oxazol-4-yl)ethoxy)benzo[b]thiophen-7-yl)propanoate (740.0 mg, yield 60.0%) as a yellow oil.

LC-MS (ESI⁺): 470.1 ([M+H]⁺).

To a solution of methyl 3-hydroxy-2-methoxy-3-(4-(2-(5-methyl-2-(phenyl-2,6-d₂)oxazol-4-yl)ethoxy)benzo[b]thiophen-7-yl)propanoate (740 mg, 1.6 mmol, 1.0 eq) in DMF (10 mL) was added con. H₂SO₄ (235.0 mg, 2.3 mmol, 1.5 eq) dropwise at room temperature. The reaction mixture was stirred at 100° C. for 14 hours and then quenched with ice-water (30 mL) and extracted with DCM (60 mL×2). The organic layer was dried over (Na₂SO₄) and concentrated to give crude product, which was triturated with EtOAc to afford methyl (Z)-2-methoxy-3-(4-(2-(5-methyl-2-(phenyl-2,6-d₂)oxazol-4-yl)ethoxy)benzo[b]thiophen-7-yl)acrylate (340.0 mg, yield 47.2%) as a yellow solid.

¹HNMR (300 MHz, CDCl₃) δ: 8.10 (d, J=8.4 Hz, 1H), 7.49 (d, J=5.5 Hz, 1H), 7.43-7.40 (m, 3H), 7.34 (d, J=5.5 Hz, 1H), 7.21 (s, 1H), 6.85 (d, J=8.4 Hz, 1H), 4.45 (t, J=8.8 Hz, 2H) 3.88 (s, 3H), 3.77 (s, 3H), 3.09 (t, J=8.8 Hz, 2H), 2.41 (s, 3H).

LC-MS (ESI⁺): 452.2([M+H]⁺).

To a solution of methyl (Z)-2-methoxy-3-(4-(2-(5-methyl-2-(phenyl-2,6-d₂)oxazol-4-yl)ethoxy)benzo[b]thiophen-7-yl)acrylate (340.0 mg, 0.8 mmol, 1.0 eq) in MeOH/THF=3:1 (16 mL) was added a solution of KOH (254.0 mg, 4.5 mmol, 6.0 eq) in water (1.1 mL). The reaction mixture was stirred at 65° C. for 1 hour and then diluted with water (20 mL), concentrated and adjusted to pH=3 with 1 N HCl. The mixture was extracted with DCM/MeOH=10:1 (50 mL×2). The organic layer was dried over Na₂SO₄, filtered and concentrated to give a crude product, which was triturated with EtOAc to afford (Z)-2-methoxy-3-(4-(2-(5-methyl-2-(phenyl-2,6-d₂)oxazol-4-yl)ethoxy)benzo[b]thiophen-7-yl)acrylic acid (200.0 mg, yield 61.0%) as a yellow solid.

¹HNMR (400 MHZ, CDCl₃) δ: 8.11 (d, J=8.4 Hz, 1H), 7.49 (d, J=5.5 Hz, 1H), 7.43 (q, J=4.1 Hz, 3H), 7.35 (d, J=5.6 Hz, 2H), 6.88 (d, J=8.5 Hz, 1H), 4.48 (t, J=6.5 Hz, 2H), 3.79 (s, 3H), 3.12 (t, J=6.5 Hz, 2H), 2.42 (s, 3H).

LC-MS (ESI⁺): 438.0 ([M+H]⁺).

A 30 mL stainless steel autoclave was charged with (Z)-2-methoxy-3-(4-(2-(5-methyl-2-(phenyl-2,6-d₂)oxazol-4-yl)ethoxy)benzo[b]thiophen-7-yl)acrylic acid (350.0 mg, 0.8 mmol, 1.0 eq), (S)-phenylethylamine (19.4 mg, 0.16 mmol, 0.2 eq), MeOH (4.2 mL), THF (2.8 mL) and Ir-cat ([((S)-DTBSIPHOX)Ir(COD)]BArF, 7.0 mg, 0.004 eq). The autoclave was sealed and the hydrogenation was stirred at 70° C. under 30 bar of hydrogen for 36 hours. The reaction solution was evaporated to dryness. The crude product was dissolved in DCM (40 mL) and washed with 1N HCl (20 mL). The organic layer was dried over Na₂SO₄, filtered and concentrated. The crude product was dissolved in isopropyl acetate at reflux and filtered. The filtrate was cooled to room temperature whereby crystallization started. The formed crystals were filtered off to afford compound 9 (142.7 mg, yield 40.6%) as a white solid.

¹HNMR (400 MHZ, CDCl₃) δ: 7.49-4.47 (m, 1H), 7.43-7.41 (m, 3H), 7.32 (d, J=5.2 Hz, 1H), 7.15 (d, J=7.6 Hz, 1H), 6.73 (d, J=8.0 Hz, 1H), 4.34 (t, J=6.4 Hz, 2H), 4.20-4.18 (m, 1H), 3.37-3.32 (m, 4H), 3.24-3.18 (m, 1H), 3.06 (t, J=6.4 Hz, 2H), 2.40 (s, 3H).

LC-MS (ESI⁺): 440.1 ([M+H]⁺).

Chiral HPLC (Chiralpak AD-3 4.6 mm*250 mm 3 μm, 90% hexanes/9.99% EtOH/0.01% TFA, 210 nm): 99.37% ee.

### Example 10: Synthesis of Compound 10

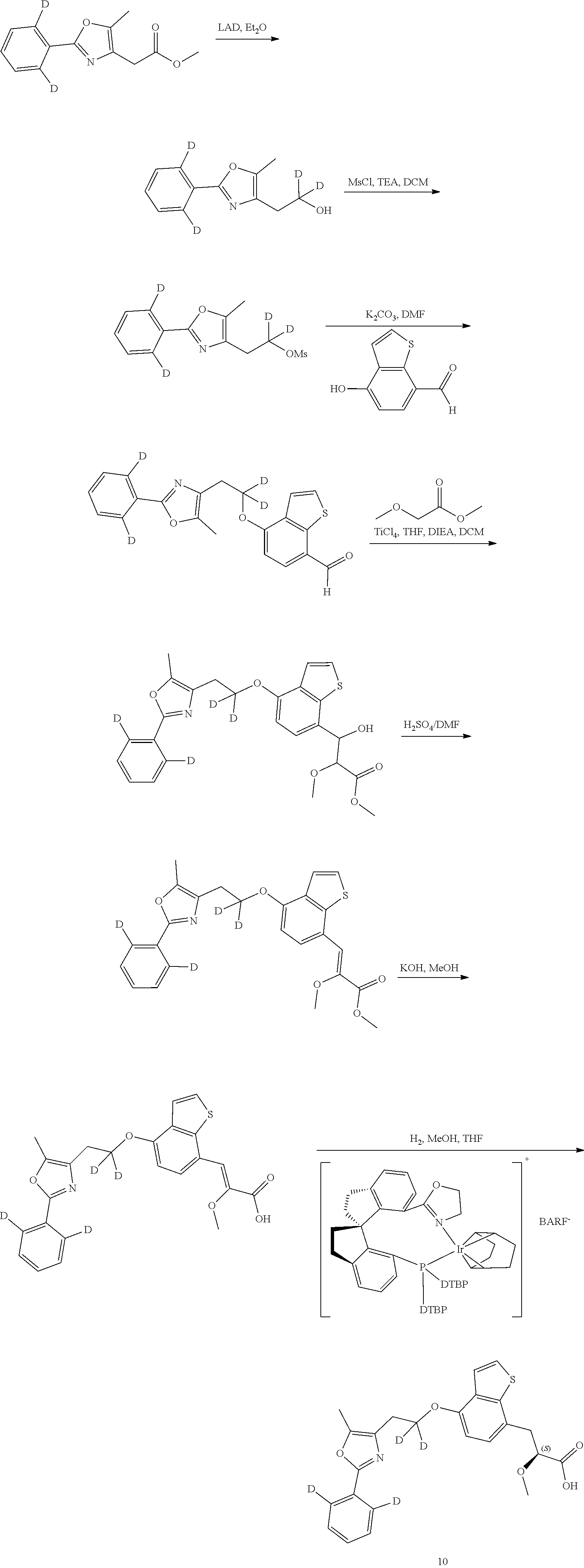

### Process Description

To an ice-cooled solution of LiAID₄ (530.0 mg, 14.0 mmol, 1.5 eq) in diethyl ether (25 mL) was added a solution of methyl 2-(5-methyl-2-(phenyl-2,6-d₂)oxazol-4-yl)acetate (2.2 g, 9.3 mmol, 1.0 eq) in diethyl ether (15 mL) dropwise and stirred at room temperature for 15 min. The reaction mixture was quenched with water (0.5 mL) and aq NaOH (15%, 0.5 mL) at 0° C. Then water (1.5 mL) was added and stirred at room temperature for 15 min. After the addition of Na₂SO₄, the mixture was filtered and concentrated to afford 2-(5-methyl-2-(phenyl-2,6-d₂)oxazol-4-yl)ethan-1,1-d₂-1-ol (1.7 g, yield 88.5%) as a white solid.

¹HNMR (400 MHZ, CDCl₃) δ: 7.45-7.41 (m, 3H), 2.71 (s, 2H), 2.34 (s, 3H).

LC-MS (ESI⁺): 208.1 ([M+H]⁺).

To a solution of 2-(5-methyl-2-(phenyl-2,6-d₂)oxazol-4-yl)ethan-1,1-d₂-1-ol (1.7 g, 8.2 mmol, 1.0 eq) in DCM (30.0 mL) was added triethylamine (1.7 g, 16.4 mmol, 2.0 eq) and methanesulfonyl chloride (1.4 g, 12.4 mmol, 1.5 eq) dropwise at 0° C. The reaction mixture was stirred at room temperature for 1 hour and then quenched with water (30 mL) and extracted with DCM (50 mL). The organic layer was dried over (Na₂SO₄) and concentrated to give 2-(5-methyl-2-(phenyl-2,6-d₂)oxazol-4-yl)ethyl-1,1-d₂ methanesulfonate (2.2 g, crude) as a yellow solid, which was used directly to the next step without further purification.

LC-MS (ESI⁺): 286.0 ([M+H]⁺).

Under argon, to a solution of 4-hydroxybenzo[b]thiophene-7-carbaldehyde (1.2 g, 6.9 mmol, 0.9 eq) in DMF (20 mL) was added K₂CO₃ (1.3 g, 9.2 mmol, 1.2 eq) at room temperature. The reaction mixture was heated to 86° C. and then a solution of 2-(5-methyl-2-(phenyl-2,6-d₂)oxazol-4-yl)ethyl-1,1-d₂ methanesulfonate (2.2 g, 7.7 mmol, 1.0 eq) in DMF (10 mL) was added. The reaction mixture was stirred at 86° C. for 5 hours and then poured into water (150 mL), and extracted with EtOAc (150 mL×2). The combined organic layer was washed with water (100 mL×2) and brine (100 mL×2). The organic layer was dried over Na₂SO₄, filtered and concentrated. The crude product was triturated with EtOAc to afford 4-(2-(5-methyl-2-(phenyl-2,6-d₂)oxazol-4-yl)ethoxy-1,1-d₂)benzo[b]thiophene-7-carbaldehyde (1.9 g, yield 66.4%) as a yellow solid, which was directly used to the next step without further purification.

¹HNMR (400 MHZ, CDCl₃) δ: 10.06 (s, 1H), 7.81 (d, J=8.0 Hz, 1H), 7.53 (s, 1H), 7.45-7.41 (m, 3H), 6.94 (d, J=8.0 Hz, 1H), 3.11 (s, 2H), 2.42 (s, 3H).

LC-MS (ESI⁺): 368.0 ([M+H]⁺).

To a solution of methyl 2-methoxyacetate (2.8 g, 26.6 mmol, 5.2 eq) in THF (20 mL) was added TiCl₄ (5.1 g, 26.6 mmol, 5.2 eq) and diisopropylethyl amine (3.7 g, 28.7 mmol, 5.6 eq) dropwise at 0° C. After 15 min, a solution of 4-(2-(5-methyl-2-(phenyl-2,6-d₂)oxazol-4-yl)ethoxy-1,1-d₂)benzo[b]thiophene-7-carbaldehyde (1.9 g, 5.1 mmol, 1.0 eq) in DCM (20 mL) was added. The reaction mixture was stirred at 0° C. for 4 hours and then quenched with water (50 mL) at 0° C. and extracted with DCM (50 mL×2). The organic layer was dried over Na₂SO₄, filtered and concentrated to give a crude product which was purified by silica gel column (eluted with petroleum ether/EtOAc=3:1) to afford methyl 3-hydroxy-2-methoxy-3-(4-(2-(5-methyl-2-(phenyl-2,6-d₂)oxazol-4-yl)ethoxy-1,1-d₂)benzo[b]thiophen-7-yl)propanoate (1.4 g, yield 58.0%) as a yellow oil.

LC-MS (ESI⁺): 472.0 ([M+H]⁺).

To a solution of methyl 3-hydroxy-2-methoxy-3-(4-(2-(5-methyl-2-(phenyl-2,6-d₂)oxazol-4-yl)ethoxy-1,1-d₂)benzo[b]thiophen-7-yl)propanoate (1.4 g, 2.9 mmol, 1.0 eq) in DMF (15 mL) was added con. H₂SO₄ (440.0 mg, 4.4 mmol, 1.5 eq) dropwise at room temperature. The reaction mixture was stirred at 100° C. for 14 hours and then quenched with ice-water (45 mL) and extracted with DCM (60 mL×2). The organic layer was dried (Na₂SO₄) and concentrated to give crude product, which was triturated with EtOAc to afford methyl (Z)-2-methoxy-3-(4-(2-(5-methyl-2-(phenyl-2,6-d₂)oxazol-4-yl)ethoxy-1,1-d₂)benzo[b]thiophen-7-yl)acrylate (600.0 mg, yield 45.6%) as a yellow solid.

¹HNMR (400 MHZ, CDCl₃) δ: 8.10 (d, J=8.4 Hz, 1H), 7.49 (d, J=5.5 Hz, 1H), 7.43-7.40 (m, 3H), 7.34 (d, J=5.5 Hz, 1H), 3.88 (s, 3H), 3.77 (s, 3H), 3.07 (s, 2H), 2.40 (s, 3H).

LC-MS (ESI⁺): 454.0([M+H]⁺).

To a solution of methyl (Z)-2-methoxy-3-(4-(2-(5-methyl-2-(phenyl-2,6-d₂)oxazol-4-yl)ethoxy-1,1-d₂)benzo[b]thiophen-7-yl)acrylate (600.0 mg, 1.3 mmol, 1.0 eq) in MeOH/THF=3:1 (20 mL) was added a solution of KOH (445.0 mg, 7.9 mmol, 6.0 eq) in water (1.5 mL). The reaction mixture was stirred at 65° C. for 1 hour and then diluted with water (30 mL), concentrated and adjusted to pH=3 with 1N HCl. The mixture was extracted with DCM/MeOH=10:1 (50 mL×2). The organic layer was dried over Na₂SO₄, filtered and concentrated to give (Z)-2-methoxy-3-(4-(2-(5-methyl-2-(phenyl-2,6-d₂)oxazol-4-yl)ethoxy-1,1-d₂)benzo[b]thiophen-7-yl)acrylic acid (550.0 mg, yield 94.8%) as a yellow solid, which was directly used to the next step without further purification.

¹HNMR (400 MHZ, CDCl₃) δ: 8.09 (d, J=8.3 Hz, 1H), 7.48 (d, J=5.5 Hz, 1H), 7.44-7.41 (m, 3H), 7.34-7.32 (m, 2H), 6.85 (d, J=8.3 Hz, 1H), 3.78 (s, 3H), 3.08 (s, 2H), 2.40 (s, 3H).

LC-MS (ESI⁺): 440.0([M+H]⁺).

A 30 mL stainless steel autoclaves was charged with (Z)-2-methoxy-3-(4-(2-(5-methyl-2-(phenyl-2,6-d₂)oxazol-4-yl)ethoxy-1,1-d₂)benzo[b]thiophen-7-yl)acrylic acid (550.0 mg, 1.0 mmol, 1.0 eq), (S)-phenylethylamine (26.0 mg, 0.2 mmol, 0.2 eq), MeOH (4.8 mL), THF (3.2 mL) and Ir-cat ([((S)-DTBSIPHOX)Ir(COD)]BArF, 8.0 mg, 0.004 eq). The autoclave was sealed and the hydrogenation was stirred at 70° C. under 30 bar of hydrogen for 36 hours. The reaction solution was evaporated to dryness. The crude product was dissolved in DCM (40 mL) and washed with 1N HCl (20 mL). The organic layer was dried over Na₂SO₄, filtered and concentrated. The crude product was dissolved in isopropyl acetate at reflux and filtered. The filtrate was cooled to room temperature whereby crystallization started. The formed crystals were filtered and dried to afford compound 10 (250.0 mg, yield 45.0%) as a white solid.

¹HNMR (400 MHZ, CDCl₃) δ: 7.48-4.47 (m, 1H), 7.43-7.42 (m, 3H), 7.31 (d, J=5.2 Hz, 1H), 7.15 (d, J=7.6 Hz, 1H), 6.72 (d, J=8.0 Hz, 1H), 4.21-4.18 (m, 1H), 3.34-3.33 (m, 4H), 3.24-3.18 (m, 1H), 3.04 (s, 2H), 2.40 (s, 3H).

LC-MS (ESI⁺): 442.1 ([M+H]⁺).

Chiral HPLC (Chiralpak AD-3 4.6 mm*250 mm 3 μm, 90% hexanes/9.99% EtOH/0.01% TFA, 210 nm): 99.81% ee.

### Example 11: Synthesis of Compound 11

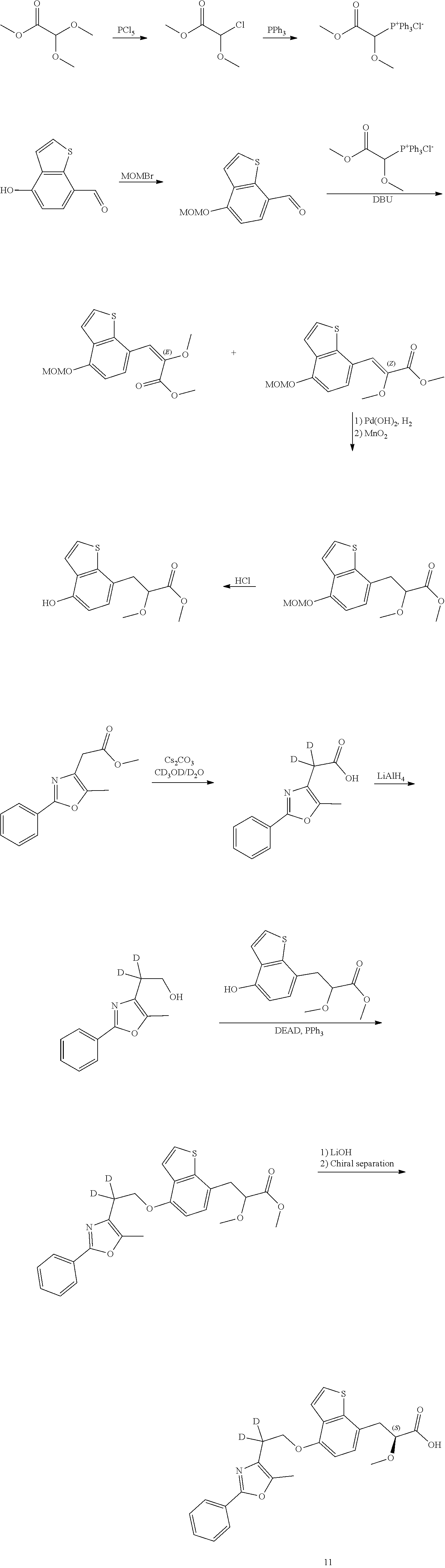

### Process Description

To a stirred solution of methyl 2,2-dimethoxyacetate (50 g, 372.77 mmol, 1.00 eq) was added PCl₅ (77.63 g, 372.77 mmol, 1.00 eq) in portions for 30 min at 0° C. under N₂ atmosphere. The resulting solution was transferred to a 350 mL seal tube, and stirred for 1.5 hours at 140° C. The mixture was allowed to cool down to room temperature. The crude product was purified by distillation under reduced pressure (10 Torr) with oil pump and the fraction was collected at 55° C. This resulted in methyl 2-chloro-2-methoxyacetate (43.0 g, 83.2%) as a colorless oil.

¹H NMR (400 MHZ, CDCl₃) δ: 5.76 (s, 1H), 3.87 (s, 3H), 3.63 (s, 3H)

To a stirred solution of methyl 2-chloro-2-methoxyacetate (20.0 g, 144.35 mmol, 1.00 eq) in dichloromethane (100 mL) was added triphenylphosphine (37.86 g, 144.35 mmol, 1.00 eq) in portions at room temperature under N₂ atmosphere. The resulting mixture was stirred overnight and then concentrated under reduced pressure and washed with Et₂O (3×100 mL). This resulted in (1,2-dimethoxy-2-oxoethyl)triphenylphosphonium chloride (45 g, 77.77%) as a light yellow solid.

To a stirred solution of 4-hydroxybenzo[b]thiophene-7-carbaldehyde (4.0 g, 22.44 mmol, 1.00 eq) and Diisopropylethylamine (11.60 g, 89.78 mmol, 4.00 eq) in dichloromethane (40 mL) was added bromo(methoxy)methane (4.21 g, 33.66 mmol, 1.50 eq) dropwise at 0° C. under N₂ atmosphere. The resulting solution was stirred for 2 hours at room temperature and then quenched with H₂O (50 mL) at 0° C. and filtered. The filter cake was washed with dichloromethane (2×50 mL). The combined organic layer was washed with aqueous NaCl (1×50 mL), dried over anhydrous Na₂SO₄. After filtration, the filtrate was concentrated under reduced pressure. The residue was purified by silica gel column chromatography, eluted with petroleum ether/ethyl acetate (10:1) to afford 4-(methoxymethoxy)benzo[b]thiophene-7-carbaldehyde (1.5 g, 30.07%) as a colorless oil.

LC-MS (ESI⁺): 223 ([M+H]⁺).

¹H NMR (400 MHZ, DMSO-d₆) δ: 10.10 (s, 1H), 8.10 (d, J 8.1 Hz, 1H), 7.88 (d, J 5.5 Hz, 1H), 7.60 (d, J 5.5 Hz, 1H), 7.28 (d, J 8.2 Hz, 1H), 5.53 (s, 2H), 3.47 (s, 3H).

To a stirred solution of 4-(methoxymethoxy)benzo[b]thiophene-7-carbaldehyde (1.5 g, 6.74 mmol, 1.00 eq) and (1,2-dimethoxy-2-oxoethyl)triphenylphosphonium chloride (21.64 g, 53.99 mmol, 8.00 eq) in tetrahydrofuran (30 mL) and CHCl₃ (30 mL) was added 1,8-Diazabicyclo[5.4.0]undec-7-ene (8.22 g, 53.99 mmol, 8.00 eq) at room temperature under N₂ atmosphere. The resulting solution was stirred for 2 hours at 60° C. The mixture was allowed to cool down to room temperature. The residue was purified by silica gel column chromatography, eluted with petroleum ether/ethyl acetate (10:1) to afford methyl (E)-2-methoxy-3-(4-(methoxymethoxy)benzo[b]thiophen-7-yl)acrylate (1.43 g, 68.7%) as a colorless oil and methyl (Z)-2-methoxy-3-(4-(methoxymethoxy)benzo[b]thiophen-7-yl)acrylate (0.30 g, 14.4%) as a colorless oil.

LC-MS (ESI⁺): 309 ([M+H]⁺).

¹H NMR (400 MHZ, DMSO-d₆) δ: 8.04 (d, J 8.4 Hz, 1H), 7.76 (d, J 5.5 Hz, 1H), 7.55 (d, J 5.5 Hz, 1H), 7.15 (d, J 8.4 Hz, 1H), 7.03 (s, 1H), 5.42 (s, 2H), 3.83 (s, 3H), 3.75 (s, 3H), 3.45 (s, 3H).

To a solution of methyl (E)-2-methoxy-3-(4-(methoxymethoxy)benzo[b]thiophen-7-yl)acrylate (1.4 g, 4.54 mmol, 1.00 eq) in methanol (140 mL) was added Pd(OH)₂ (20 wt % on carbon, 0.14 g) under nitrogen atmosphere in a 250 mL Pressure Reactor. The mixture was hydrogenated at room temperature for 40 hours under 40 atm hydrogen atmosphere. The reaction mixture was filtered through a Celite pad and concentrated under reduced pressure. The residue was dissolved in dichloroethane (28 mL) and manganese dioxide (7.89 g, 90.80 mmol, 20 eq) was added. The mixture was stirred for 6 hours at 80° C. under N₂ atmosphere. The mixture was allowed to cool down to room temperature and filtered. The filter cake was washed with dichloromethane (2×50 mL). The filtrate was concentrated under reduced pressure. The residue was purified by silica gel column chromatography, eluted with petroleum ether/ethyl acetate (10:1) to afford methyl methyl 2-methoxy-3-(4-(methoxymethoxy)benzo[b]thiophen-7-yl)propanoate (750.0 mg, 53.2%) as a colorless oil.

LC-MS (ESI⁺): 328 ([M+NH₄]⁺).

¹H NMR (400 MHZ, DMSO-d₆) δ: 7.69 (d, J 5.5 Hz, 1H), 7.50 (d, J 5.5 Hz, 1H), 7.14 (d, J 8.1 Hz, 1H), 6.99 (d, J 8.0 Hz, 1H), 5.34 (s, 2H), 4.21 (dd, J 7.9, 5.1 Hz, 1H), 3.66 (s, 3H), 3.43 (s, 3H), 3.23 (s, 3H), 3.20-3.03 (m, 2H).

To a stirred solution of methyl 2-methoxy-3-(4-(methoxymethoxy)benzo[b]thiophen-7-yl)propanoate (750 mg, 1 equiv) in dioxane (7.5 mL) was added HCl in 1,4-dioxane (4M, 7.5 mL) at room temperature under N₂ atmosphere. The resulting solution was stirred for 1 hour at room temperature. The resulting mixture was concentrated under reduced pressure. This resulted in methyl 3-(4-hydroxybenzo[b]thiophen-7-yl)-2-methoxypropanoate (600.0 mg, 93.2%) as a light brown oil.

LC-MS (ESI⁺): 289 ([M+Na]⁺).

¹H NMR (400 MHZ, DMSO-d₆) δ: 9.29 (s, 1H), 8.03-7.99 (m, 1H), 7.97 (d, J 5.5 Hz, 1H), 7.52 (d, J 7.9 Hz, 1H), 7.22 (d, J 7.8 Hz, 1H), 4.69-4.59 (m, 1H), 4.14 (d, J 1.4 Hz, 3H), 3.74 (d, J 1.4 Hz, 3H), 3.61 (qd, J 14.4, 6.7 Hz, 2H).

To a stirred solution of methyl 2-(5-methyl-2-phenyloxazol-4-yl)acetate (500.0 mg, 2.16 mmol, 1.00 eq) in CD₃OD (5 mL, 112.293 mmol, 51.94 eq) and D₂O (5 mL, 274.621 mmol, 127.01 eq) was added Cs₂CO₃ (2113.4 mg, 6.48 mmol, 3.00 eq) at room temperature under N₂ atmosphere. The resulting mixture was stirred for 16 hours at room temperature and then concentrated under reduced pressure. The residue was dissolved in new CD₃OD (5 mL) and D₂O (5 mL) and stirred for 24 hours at room temperature. Stirring longer time, the deuterium value was not increased. The resulting mixture was diluted with H₂O (50 mL), extracted with methyl tert-butylether (1×30 mL) and aqueous layers were combined. The combined aqueous layer was acidified to pH=3 with 1N HCl (aq). The resulting mixture was extracted with EtOAc (3×30 mL). The combined organic layer was washed with aqueous NaCl (1×30 mL), dried over anhydrous Na₂SO₄. After filtration, the filtrate was concentrated under reduced pressure. This resulted in 2-(5-methyl-2-phenyloxazol-4-yl)acetic-2,2-d₂ acid (420 mg, 88.60%) as a white solid.

LC-MS (ESI⁺): 220 ([M+H]⁺).

D/H ratio by LC-MS (ESI⁺): 92.46%.

To a stirred solution of LiAlH₄ (58.8 mg, 1.55 mmol, 2.00 eq) in tetrahydrofuran (8.5 mL) was added 2-(5-methyl-2-phenyloxazol-4-yl)acetic-2,2-d₂ acid (170.0 mg, 0.77 mmol, 1.00 eq) in tetrahydrofuran (1.5 mL) dropwise at 0° C. under N₂ atmosphere. The resulting mixture was stirred for 2 hours at 0° C. and then diluted with tetrahydrofuran (20 mL), quenched with Na₂SO₄·10H₂O at 0° C. The resulting mixture was filtered, the filter cake was washed with tetrahydrofuran (2×20 mL). The filtrate was concentrated under reduced pressure. This resulted in 2-(5-methyl-2-phenyloxazol-4-yl)ethan-2,2-d₂-1-ol (155 mg, 97.39%) as a light yellow solid.

LC-MS (ESI⁺): 206 ([M+H]⁺).

D/H ratio by LC-MS (ESI⁺): 92.12%.

To a stirred solution of 2-(5-methyl-2-phenyloxazol-4-yl)ethan-2,2-d₂-1-ol (60.0 mg, 0.29 mmol, 1.00 eq), methyl 3-(4-hydroxybenzo[b]thiophen-7-yl)-2-methoxypropanoate (51.3 mg, 0.19 mmol, 0.66 eq) and PPh₃ (153.3 mg, 0.58 mmol, 2.00 eq) in tetrahydrofuran (6 mL) was added diethyl azodicarboxylate (101.8 mg, 0.58 mmol, 2.00 eq) in tetrahydrofuran (0.5 mL) dropwise at 0° C. under N₂ atmosphere. The reaction was stirred for 2 hours at room temperature, and then quenched with H₂O (20 mL). The resulting mixture was extracted with EtOAc (3×20 mL). The combined organic layer was washed with aqueous NaCl (20 mL), dried over anhydrous Na₂SO₄. After filtration, the filtrate was concentrated under reduced pressure. The residue was purified by Prep-TLC (petroleum ether/ethyl acetate, 4:1) to afford methyl 2-methoxy-3-(4-(2-(5-methyl-2-phenyloxazol-4-yl)ethoxy-2,2-d₂)benzo[b]thiophen-7-yl)propanoate (20.0 mg, 15.0%) as a light yellow solid.

LC-MS (ESI⁺): 454 ([M+H]⁺).

To a stirred solution of methyl 2-methoxy-3-(4-(2-(5-methyl-2-phenyloxazol-4-yl)ethoxy-2,2-d₂)benzo[b]thiophen-7-yl)propanoate (20.0 mg, 0.044 mmol, 1.00 eq) in tetrahydrofuran (4 mL) and H₂O (2 mL) was added LiOH (4.2 mg, 0.17 mmol, 4.00 eq) at 0° C. under N₂ atmosphere. After stirred for 1 hour at room temperature, the resulting mixture was diluted with H₂O (10 mL) and acidified to pH=4 with 1N HCl (aq). The mixture was extracted with EtOAc (3×20 mL). The combined organic layer was washed with aqueous NaCl (20 mL), dried over anhydrous Na₂SO₄. After filtration, the filtrate was concentrated under reduced pressure. The crude product (18 mg) was purified by prep-chiral-HPLC with the following conditions (Column: CHIRALPAK IG, 2*25 cm, 5 μm; Mobile Phase A: HEX: MTBE=1: 1(0.2% FA)-HPLC, Mobile Phase B: IPA—HPLC; Flow rate: 20 mL/min; Gradient: 6% B to 6% B in 15.5 min; Wave Length: 220/254 nm; RT1(min): 11.8; RT2(min): 14.1; Sample Solvent: EtOH—HPLC; Injection Volume: 0.3 mL; Number Of Runs: 7) to afford earlier fraction (3.2 mg, 16.5%) as a white solid and later fraction (3.1 mg, 15.9%) as a white solid.

LC-MS (ESI⁺): 440 ([M+H]⁺).

D/H ratio by LC-MS (ESI⁺): 92.17%.

Chiral HPLC (Chiralpak AD-3 4.6 mm*250 mm 3 μm, 90% hexanes/9.99% EtOH/0.01% TFA, 210 nm): >99.99% ee.

¹H NMR (400 MHZ, Chloroform-d) δ: 8.38 (d, J=7.5 Hz, 2H), 7.68-7.58 (m, 3H), 7.40 (d, J=5.5 Hz, 1H), 7.34 (d, J=5.4 Hz, 1H), 7.23 (m, 1H), 7.15 (d, J=7.8 Hz, 1H), 6.81 (d, J=7.9 Hz, 1H), 4.58 (s, 2H), 4.18 (dd, J=8.1, 4.3 Hz, 1H), 3.35 (dd, J=14.6, 4.2 Hz, 1H), 3.32 (s, 3H), 3.18 (dd, J=14.7, 8.1 Hz, 1H), 2.53 (s, 3H).

### Example 12: Synthesis of Compound 12

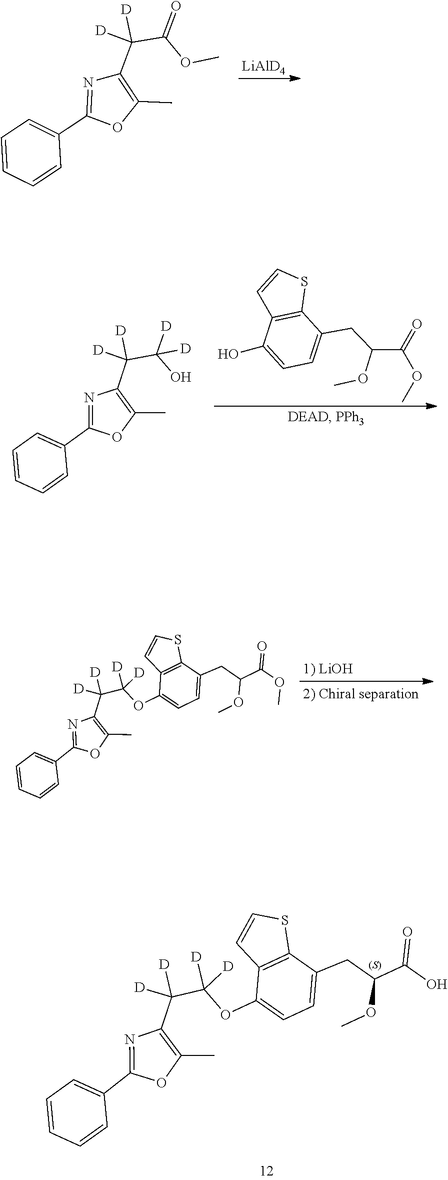 [0491] Process Description

To a stirred solution of LiAID₄ (53.9 mg, 1.28 mmol, 2 eq) in THF (7.5 mL) was added methyl 2-(5-methyl-2-phenyloxazol-4-yl)acetate-d₂ (150.0 mg, 0.64 mmol, 1.00 eq) in THF (2.5 mL) dropwise at 0° C. under N₂ atmosphere. The resulting solution was stirred for 1 hour at 0° C. and then diluted with THF (20 mL) and quenched with Na₂SO₄·10H₂O at 0° C. The resulting solution was dried over Na₂SO₄ and filtered. The filter cake was washed with THF (2×20 mL). The filtrate was concentrated under reduced pressure. This resulted in 2-(5-methyl-2-phenyloxazol-4-yl)ethan-1,1,2,2-d₄-1-ol (140 mg, 105.04%, crude) as a light yellow solid.

LC-MS (ESI⁺): 208 ([M+H]⁺).

D/H ratio by LC-MS (ESI⁺): 95.59%.

To a stirred solution of 2-(5-methyl-2-phenyloxazol-4-yl)ethan-1,1,2,2-d₄-1-ol (60.0 mg, 0.28 mmol, 1.00 eq), methyl 3-(4-hydroxybenzo[b]thiophen-7-yl)-2-methoxypropanoate (50.8 mg, 0.19 mmol, 0.66 eq) and PPh₃ (151.8 mg, 0.57 mmol, 2 eq) in THF (3 mL) were added diethyl azodicarboxylate (100.83 mg, 0.578 mmol, 2 eq) in THF (0.5 mL) dropwise at 0° C. under N₂ atmosphere. The resulting solution was stirred for 2 hours at room temperature and then quenched with H₂O (10 mL) and extracted with EtOAc (3×20 mL). The combined organic layer was washed with aqueous NaCl (1×20 mL), dried over anhydrous Na₂SO₄. After filtration, the filtrate was concentrated under reduced pressure. The residue was purified by Prep-TLC (petroleum ether/ethyl acetate, 4:1) to afford methyl 2-methoxy-3-(4-(2-(5-methyl-2-phenyloxazol-4-yl)ethoxy-1,1,2,2-d₄)benzo[b]thiophen-7-yl)propanoate (28 mg, 21.23%) as a light yellow solid.

LC-MS (ESI⁺): 456 ([M+H]⁺).

D/H ratio by LC-MS (ESI⁺): 95.41%.

To a stirred solution of methyl 2-methoxy-3-(4-(2-(5-methyl-2-phenyloxazol-4-yl)ethoxy-1,1,2,2-d₄)benzo[b]thiophen-7-yl)propanoate (28 mg, 0.061 mmol, 1.00 eq) in tetrahydrofuran (4 mL) and H₂O (2 mL) was added LiOH (5.8 mg, 0.244 mmol, 4.00 eq) at 0° C. under N₂ atmosphere. After stirred for 2 hours at room temperature, the resulting mixture was diluted with H₂O (10 mL) and acidified pH to 4 with 1N HCl (aq). The resulting mixture was extracted with EtOAc (3×20 mL). The combined organic layer was washed with aqueous NaCl (20 mL), dried over anhydrous Na₂SO₄. After filtration, the filtrate was concentrated under reduced pressure. The crude product (22 mg) was purified by prep-chiral-HPLC with the following conditions (Column: CHIRALPAK IG, 2*25 cm, 5 μm; Mobile Phase A: HEX: MtBE=1:1 (0.2% FA)-HPLC, Mobile Phase B: IPA—HPLC; Flow rate: 20 mL/min; Gradient: 6% B to 6% B in 16 min; Wave Length: 220/254 nm; RT1(min): 11.7; RT2(min): 14.3; Sample Solvent: EtOH—HPLC; Injection Volume: 0.3 mL; Number Of Runs: 7) to afford earlier fraction (8.7 mg, 32.06%) as a white solid and later fraction (7.3 mg, 26.90%) as a white solid. LC-MS (ESI⁺): 442 ([M+H]⁺).

D/H ratio by LC-MS (ESI⁺): 95.57%.

Chiral HPLC (Chiralpak AD-3 4.6 mm*250 mm 3 μm, 90% hexanes/9.99% EtOH/0.01% TFA, 210 nm): >99.99% ee.

¹H NMR (400 MHZ, CDCl₃) δ: 8.06 (d, J=5.7 Hz, 2H), 7.53-7.44 (m, 4H), 7.35 (d, J=5.5 Hz, 1H), 7.17 (d, J=7.9 Hz, 1H), 6.78 (d, J=8.0 Hz, 1H), 4.22 (dd, J=8.0, 4.4 Hz, 1H), 3.43-3.35 (m, 1H), 3.36 (s, 3H), 3.22 (dd, J=14.7, 8.0 Hz, 1H), 2.45 (s, 3H).

### Example 13: Evaluation of Compounds on PPARα/PPARγ Activity with Luciferase Reporter System

HEK293T cell culture followed ATCC culture guides. Experiments were performed when cells were in the exponential phase of growth. Total 6×10⁶ cells were seeded into a 60 mm cell culture dish and cultured overnight in 37° C. and 5% CO₂. Lipofectamine® 3000, the transfection reagent, was mixed with plasmid combination (a mixture of pGL4.35 [luc2P/9XGAL4 UAS/Hygro], pBIND-RXRα, and pBIND-PPPARα or a mixture of pGL4.35 [luc2P/9XGAL4 UAS/Hygro], pBIND-RXRα, and pBIND-PPPARγ), and then added to the dish. After incubated for 5 hours at 37° C. and 5% CO₂, the cells was trypsinzied and seeded into a 384-well assay plate, followed by incubation with the test compound at the serial concentrations for overnight in 37° C. and 5% CO₂. At the following day, Cells were lyzed and luciferase was activated with the Steady-Glo™ Luciferase Assay System. Luminescence signals from the luciferase assay were measured by Envision HTS/2105. Since peroxisome proliferator-activated transcription regulates the expression of luciferase, the agonist activities of the test compounds can be qualified by the luminescence intensity. The EC₅₀ value of test compounds was calculated for the PPARα/γ agonistic potency by using Graphpad 8.0, and the results were shown in Table 1. Selectivity of compounds towards PPARα or PPARγ was expressed as PPARγ EC₅₀/PPARα EC₅₀.

TABLE 1
Results of PPARa/y agonism testing

| Compound ID | PPARα EC₅₀ (nM) | PPARγ EC₅₀ (nM) | Selectivity* |
|---|---|---|---|
| Aleglitazar | 3.14 | 3.36 | 1.07 |
| Compound 1 | 2.56 | 2.27 | 0.89 |
| Compound 2 | 2.52 | 3.81 | 1.51 |
| Compound 3 | 2.24 | 2.64 | 1.18 |
| Compound 4 | 2.72 | 2.94 | 1.08 |
| Compound 5 | 7.36 | 2.17 | 0.29 |
| Compound 6 | 4.98 | 1.41 | 0.28 |
| Compound 7 | 6.02 | 1.89 | 0.31 |
| Compound 8 | 6.62 | 1.91 | 0.29 |
| Compound 9 | 6.83 | 2.16 | 0.32 |
| Compound 10 | 7.83 | 2.71 | 0.35 |
| Compound 11 | 7.72 | 4.26 | 0.55 |
| Compound 12 | 8.34 | 5.56 | 0.67 |

*Selectivity = PPARγ EC₅₀ (nM)/PPARα EC₅₀ (nM)

### Example 14: The Effect of the Compounds Provided Herein on the Rat Model of Hyperlipidemia Induced by High Cholesterol Diet

### 14.1 Experimental Materials:

42 male Sprague-Dawley rats aged 6-8 weeks; source: SPF (Beijing) Biotechnology Co., Ltd.; animal certificate number: 110324201104469613.

### 14.2 Experimental Methods:

14.2.1 High cholesterol diet (ASHF4) was used to induce an animal model of hyperlipidemia in the SD rats.

14.2.2 The male SD rats were fed with high cholesterol diet (ASHF4, Dyet, China) for 14 days. On the day before the start of the administration (Day 0), the animals were divided into 7 groups based on body weights and serum indicators and were continually fed with the high-cholesterol diet. The treatment groups were orally dosed with either compound or vehicle while continuing the high-cholesterol diet for a total of 1 week. The animals were weighed daily before the treatment, and the compounds were given at 9:00-9:30 in the morning based on the body weight of the day. The specific grouping and dosing regimen were shown in Table 2.

TABLE 2
Grouping and dosing of the experimental animals

| | | | Dosing | | Dosing | Numbers |
|---|---|---|---|---|---|---|
| | | Dosage | volume | Dosing | frequen- | of |
| Group | Drugs | (mg/kg) | (mL/kg) | route | cy | Animals |
| 1 | High | — | 5 | Oral | QD × 7 | 6 |
| | Cholesterol | | | | days | |
| | Diet + | | | | | |
| | Vehicle | | | | | |
| 2 | High | 0.2 | 5 | Oral | QD × 7 | 6 |
| | Cholesterol | | | | days | |
| | Diet + | | | | | |
| | Aleglitazar | | | | | |
| 3 | High | 0.6 | 5 | Oral | QD × 7 | 6 |
| | Cholesterol | | | | days | |
| | Diet + | | | | | |
| | Aleglitazar | | | | | |
| 4 | High | 0.2 | 5 | Oral | QD × 7 | 6 |
| | Cholesterol | | | | days | |
| | Diet + | | | | | |
| | Compound 2 | | | | | |
| 5 | High | 0.6 | 5 | Oral | QD × 7 | 6 |
| | Cholesterol | | | | days | |
| | Diet + | | | | | |
| | Compound 2 | | | | | |
| 6 | High | 0.2 | 5 | Oral | QD × 7 | 6 |
| | Cholesterol | | | | days | |
| | Diet + | | | | | |
| | Compound 4 | | | | | |
| 7 | High | 0.6 | 5 | Oral | QD × 7 | 6 |
| | Cholesterol | | | | days | |
| | Diet + | | | | | |
| | Compound 4 | | | | | |

Formulation. The formulation was prepared twice weekly. 1. Vehicle: 0.5% sodium carboxymethyl cellulose. 5g sodium carboxymethyl cellulose was added into 900 ml ddH₂O and stirred until dissolved completely, then filled into 1000 ml with ddH₂O. 2. Working solution for 0.6 mg/kg administration: 0.12 mg/ml working solution. 12 mg compound was added into 100 ml of 0.5% sodium carboxymethyl cellulose, and then vortexed until well suspended. 3. Working solution for 0.2 mg/kg administration: 0.04 mg/ml working solution. 30 ml of 0.12 mg/ml compound solution was mixed with 60 ml of 0.5% sodium carboxymethyl cellulose, and then vortexed until well suspended.

14.2.3 Animal blood was collected, and serum was separated for the analysis of serum lipid indicators on the day before the treatment and at the end of the last day for the experiment.

14.2.4 Serum indicators of triglyceride (TG) and free fatty acid (NEFA) were determined by an automatic blood biochemical analyzer.

### 14.3 Results:

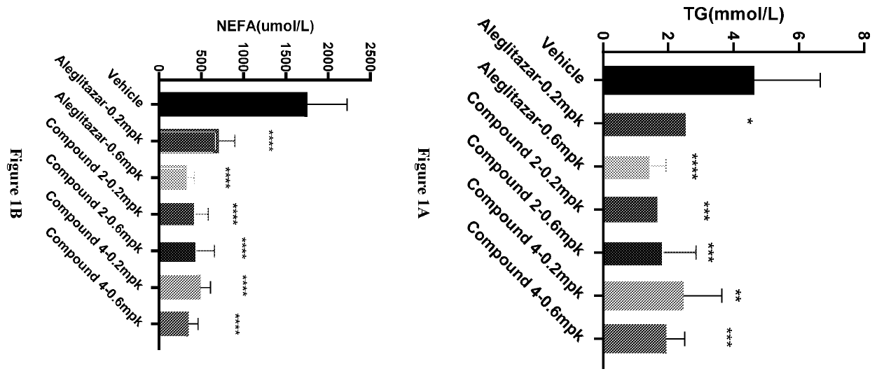

Serum lipid analysis showed that, after 7 days of treatment (Day 8), when compared with the vehicle group, Aleglitazar, Compound 2, and Compound 4 can all significantly reduce serum TG and NEFA levels at dosage levels of both 0.2 mg/kg and 0.6 mg/kg. The animal serum TG and NEFA were shown in FIGS. 1A and 1B, and their numerical values were shown in Table 3 and 4, respectively. During the experiment, there were no abnormal clinical observations. For comparative purposes, the GraphPad 8.0 software package was used to carry out T-test statistical analysis on the results of each compound at a dosage of 0.2 mg/kg. At dose level of 0.2 mg/kg, Compound 2 can significantly reduce TG and NEFA (P<0.05), while Compound 4 can significantly reduce NEFA (P<0.05), when compared with Aleglitazar.

TABLE 3
Changes of serum TG in each
group (mean ± standard deviation)

| | | TG(mmol/L) | |
|---|---|---|---|
| Group | Dosing Regimen | Day 0 | Day 8 |
| 1 | High Cholesterol Diet + | 4.00 ± 1.75 | 4.63 ± 2.02 |
| | Vehicle | | |
| 2 | High Cholesterol Diet + | 4.24 ± 1.18 | 2.86 ± 0.86* |
| | Aleglitazar | | |
| 3 | High Cholesterol Diet + | 4.09 ± 2.17 | 1.47 ± 0.46**** |
| | Aleglitazar | | |
| 4 | High Cholesterol Diet + | 4.27 ± 1.61 | 1.71 ± 0.72*** |
| | Compound 2¹ | | |
| 5 | High Cholesterol Diet + | 4.11 ± 1.10 | 1.86 ± 0.99*** |
| | Compound 2 | | |
| 6 | High Cholesterol Diet + | 4.22 ± 1.71 | 2.46 ± 1.18** |
| | Compound 4 | | |
| 7 | High Cholesterol Diet + | 4.00 ± 1.71 | 1.93 ± 0.58*** |
| | Compound 4 | | |

Note:
Each group of data was analyzed by GraphPad 8.0 software package, and the statistical method is One-way ANOVA. Compared with the first group,
*p < 0.05,
** p < 0.01,
***p < 0.001,
**** p < 0.0001.
¹TG of Group 4 (compound 2, 0.2 mg/kg) was statistically significantly lower (p < 0.05) when compared with that of Group 2 (Aleglitazar, 0.2 mg/kg)

TABLE 4
Changes of serum NEFA in
each group (mean ± standard deviation)

| | | NEFA(umol/L) |
|---|---|---|
| Group | Dosing Regimen | Day 8 |
| 1 | High Cholesterol Diet + Vehicle | 1753.50 ± 472.12 |
| 2 | High Cholesterol Diet + Aleglitazar | 706.00 ± 190.15**** |
| 3 | High Cholesterol Diet + Aleglitazar | 327.83 ± 90.73**** |
| 4 | High Cholesterol Diet + Compound 2¹ | 431.50 ± 151.63**** |
| 5 | High Cholesterol Diet + Compound 2 | 444.33 ± 119.12**** |
| 6 | High Cholesterol Diet + Compound 4¹ | 490.33 ± 113.61**** |
| 7 | High Cholesterol Diet + Compound 4 | 347.83 ± 113.61**** |

Note:
Each group of data was analyzed by GraphPad 8.0 software package, and the statistical method is One-way ANOVA. Compared with the first group,
****p < 0.0001.
¹NEFA of Groups 4 (compound 2, 0.2 mg/kg) and 6 (compound 4, 0.2 mg/kg) were statistically significantly lower (p < 0.05) when compared with that of Group 2 (Aleglitazar, 0.2 mg/kg).

### Example 15: Effect of 7-Day Repeated Oral Gavage on the Body Weights of ICR Mice

### 15.1 Experimental Materials:

70 male ICR mice aged 6-8 weeks; source: Laboratory Animal Business Department, Shanghai Institute of Planned Parenthood Research.

### 15.2 Methods:

After 3 days of acclimatization, ICR mice were grouped according to their body weights. The day of grouping is designated as Day 0. After grouping, they were dosed with either vehicle or compound by oral gavage for 7 consecutive days, once a day. The dosage and the grouping were shown in Table 5. The animals were weighed and recorded daily.

TABLE 5
Grouping and dosing of the experimental animals

| | | | Dosing | Dosing | Concen- | Number |
|---|---|---|---|---|---|---|
| | | Dosage | frequen- | volume | tration | of |
| Group | Test agent | (mg/kg) | cy | (ml/kg) | (mg/mL) | Animals |
| 1 | Vehicle^{a} | 0 | QD × 7 | 10 | 0 | 10 |
| | | | days | | | |
| 2 | Aleglitazar | 0.2 | QD × 7 | 10 | 0.02 | 10 |
| | | | days | | | |
| 3 | Aleglitazar | 1 | QD × 7 | 10 | 0.1 | 10 |
| | | | days | | | |
| 4 | Compound 2 | 0.2 | QD × 7 | 10 | 0.02 | 10 |
| | | | days | | | |
| 5 | Compound 2 | 1 | QD × 7 | 10 | 0.1 | 10 |
| | | | days | | | |
| 6 | Compound 4 | 1 | QD × 7 | 10 | 0.1 | 10 |
| | | | days | | | |
| 7 | Compound | 1 | QD × 7 | 10 | 0.1 | 10 |
| | 10 | | days | | | |

^{a}0.5% sodium carboxymethyl cellulose aqueous solution

Formulation. The formulation was prepared twice weekly. 1. Vehicle: 0.5% sodium carboxymethyl cellulose. 2.5 g sodium carboxymethyl cellulose was weighed and mixed with 500 ml ddH₂O until dissolved completely. 2. Working solution for 1 mg/kg administration: 0.1 mg/ml working solution. 3 mg of compound was added into 30 ml of 0.5% sodium carboxymethyl cellulose, and then vortexed until well suspended. 3. Working solution for 0.2 mg/kg administration: 0.02 mg/ml working solution. 6 ml of 0.1 mg/ml compound solution was mixed with 24 ml of 0.5% sodium carboxymethyl cellulose, and then stirred until well suspended.

### 15.3 Results:

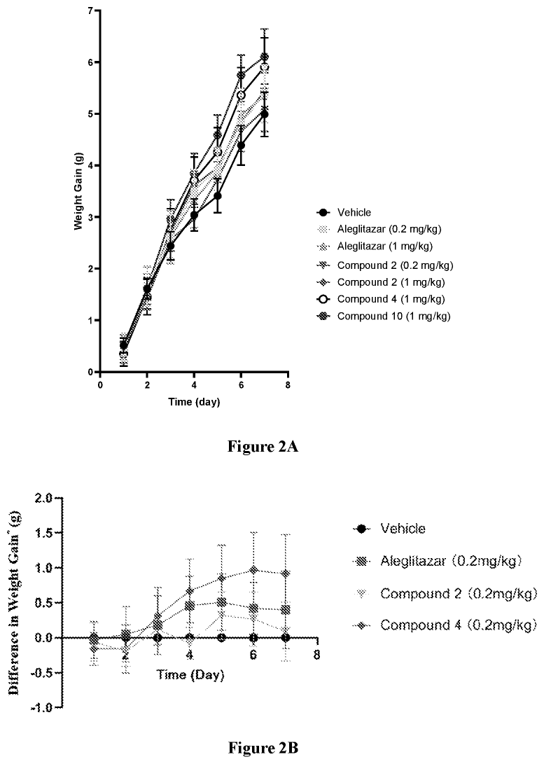

The effects of the compounds on the body weight changes of ICR mice were shown in Table 6 and FIG. 2A. During the experiment, the body weights of the animals gradually increased over time. The daily average weight gain of Aleglitazar groups at a dosage of 0.2 mg/kg (Day 4 and Day 5) and 1 mg/kg (Day 6) was significantly higher than that of the vehicle group. Compared with the vehicle group, the daily weight gain of Compound 2 group at a low dosage of 0.2 mg/kg had no significant difference throughout the study. The average daily weight gain of Compound 2 group at a high dosage of 1 mg/kg (Day 4 and 5) was significantly higher than that of the vehicle group. The weight gain of Compounds 4 and 10 at a dosage of 1 mg/kg was significantly higher than that of the vehicle group from Day 4 to Day 7, respectively. The full data was shown in Table 6. For the comparative purpose, the net weight gain of the treatment, which was derived from subtracting the average weight gain of the vehicle group from that of each treatment group, was calculated and shown in FIG. 2B.

TABLE 6
Changes in body weight gain of each group of animals compared with Day 0

| Compound | Weight Gain (g) n = 10 | | | | | | |
|---|---|---|---|---|---|---|---|
| ID | Day 1 | Day 2 | Day 3 | Day 4 | Day 5 | Day 6 | Day 7 |
| Vehicle | 0.51 ± 0.14 | 1.61 ± 0.2 | 2.44 ± 0.24 | 3.04 ± 0.31 | 3.4 ± 0.33 | 4.4 ± 0.38 | 5.0 ± 0.43 |
| Aleglitazar | 0.48 ± 0.27 | 1.66 ± 0.39 | 2.62 ± 0.42 | 3.50 ± 0.42* | 3.92 ± 0.4* | 4.81 ± 0.37 | 5.39 ± 0.56 |
| (0.2 mg/kg) | | | | | | | |
| Aleglitazar | 0.5. ± 0.2 | 1.64 ± 0.26 | 2.61 ± 0.51 | 3.33 ± 0.54 | 3.86 ± 0.49 | 4.98 ± 0.52* | 5.34 ± 0.50 |
| (1 mg/kg) | | | | | | | |
| Compound 2 | 0.45 ± 0.28 | 1.42 ± 0.22 | 2.56 ± 0.23 | 2.96 ± 0.23 | 3.73 ± 0.34 | 4.66 ± 0.39 | 5.08 ± 0.42 |
| (0.2 mg/kg) | | | | | | | |
| Compound 2 | 0.4 ± 0.17 | 1.57 ± 0.26 | 2.71 ± 0.19 | 3.61 ± 0.19** | 3.96 ± 0.29* | 4.84 ± 0.4 | 5.44 ± 0.36 |
| (1 mg/kg) | | | | | | | |
| Compound 4 | 0.35 ± 0.24 | 1.45 ± 0.34 | 2.75 ± 0.41 | 3.71 ± 0.46** | 4.26 ± 0.47**** | 5.36 ± 0.54**** | 5.91 ± 0.56*** |
| (1 mg/kg) | | | | | | | |
| Compound | 0.34 ± 0.17 | 1.63 ± 0.41 | 2.96 ± 0.37* | 3.85 ± 0.38**** | 4.59 ± 0.39**** | 5.75 ± 0.39**** | 6.11 ± 0.53**** |
| 10 (1 mg/kg) | | | | | | | |

Note
Each group of data was analyzed by GraphPad 8.0 software package, and the statistical method is One-way ANOVA. Compared with vehicle, *p < 0.05, **p < 0.01, ***p < 0.001, ****p < 0.0001.

### Example 16: The Pharmacodynamics Study of the Compounds Provided Herein on the db/db Type 2 Diabetes Model

### 16.1 Experimental Materials:

| Species | db/db mice | wild mice |
|---|---|---|
| Grade | SPF animal | SPF animal |
| Age when ordered | 6 weeks | 6 weeks |
| Age at the beginning | 8 weeks | 8 weeks |
| of the experiment | | |
| Weight range | 35~45 g | 18~25 g |
| Gender | Male | Male |
| Supplier | Jiangsu | Jiangsu |
| | GemPharmatech, | GemPharmatech, |
| | Co., Ltd. | Co., Ltd. |
| Address of the supplier | Jiangsu, China | Jiangsu, China |
| Identification method | Kept a mouse | Kept a mouse |
| | in a single | in a single |
| | numbered cage | numbered cage |
| Number of ordered | 55 | 8 |
| animals | | |
| Number of used | 45 | 6 |
| animals | | |

### 16.2 Experimental Methods:

16.2.1 Grouping in the experiment: 6 Wild mice were used as the control group (Group 1). 45 db/db mice were evenly divided into 5 groups before the initiation of treatment based on the body weights, serum triglyceride (TG) levels, and random blood glucose levels.

16.2.2 Formulation. The formulation was prepared twice weekly. 1. Vehicle: 0.5% sodium carboxymethyl cellulose. 2.5g sodium carboxymethyl cellulose was weighed and mixed with 500 ml ddH₂O until dissolved completely. 2. Working solution for 1 mg/kg administration: 0.1 mg/ml working solution. 3 mg of compound was added into 30 ml of 0.5% sodium carboxymethyl cellulose, and then vortexed until well suspended. 3. Working solution for 0.2 mg/kg administration: 0.02 mg/ml working solution. 6 ml of 0.1 mg/ml compound solution was mixed with 24 ml of 0.5% sodium carboxymethyl cellulose, and then stirred until well suspended. 4. Working solution for 0.05 mg/kg administration: 0.005 mg/ml working solution. 6 ml of 0.02 mg/ml compound solution was mixed with 18 ml of 0.5% sodium carboxymethyl cellulose, and then stirred until well suspended.

16.2.3 Dosing: The animals were orally dosed according to their grouping: Vehicle (Group 2), Aleglitazar at the dosage level of 0.2 mg/kg (Group 3), and Compound 2 at dosage levels of either 0.05 mg/kg (Group 4), 0.2 mg/kg (Group 5), or 1 mg/kg (Group 6). All animals were weighed daily before each dosing and treated daily for 14 consecutive days.

16.2.4 The study results include daily body weights throughout the study; serum TG levels before dosing at the 6^{th} and 12^{th} day of the study; the random blood glucose levels before dosing at the 7^{th} and 14^{th} day of the study; and results from oral glucose tolerance test (OGTT) performed at the 14^{th} day of the study.

### 16.3 Data Analysis:

All the data was imported into an Excel file and presented as Mean±SEM. Graphpad Prism 7.0 software was used for data statistical analysis by One-way or Two-way ANOVA, with P<0.05 as the criterion of significant differences.

### 16.4 Experimental Results:

Both Aleglitazar and all groups of Compound 2 significantly decreased the level of lipid, free fatty acid and blood glucose, and significantly increased body weight when compared with vehicle group.

### 16.4.1 Animal's Body Weight

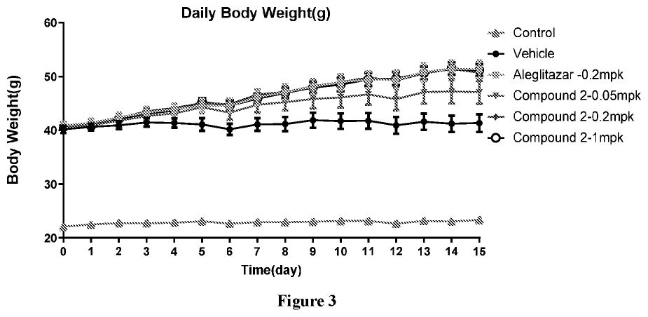

The body weight changes of db/db model animals treated with Aleglitazar and different dosages of Compound 2 are shown in FIG. 3. As shown in FIG. 3, animals' body weight in the dosage group of Aleglitazar (0.2 mg/kg) and dosage groups of Compound 2 (0.05 mg/kg, 0.2 mg/kg, 1 mg/kg) gradually increased over time during the experiment, and the average daily body weights (from Day 10 to Day 15) were all significantly higher than those of the vehicle group.

### 16.4.2 Animals' Blood Biochemical Indicators

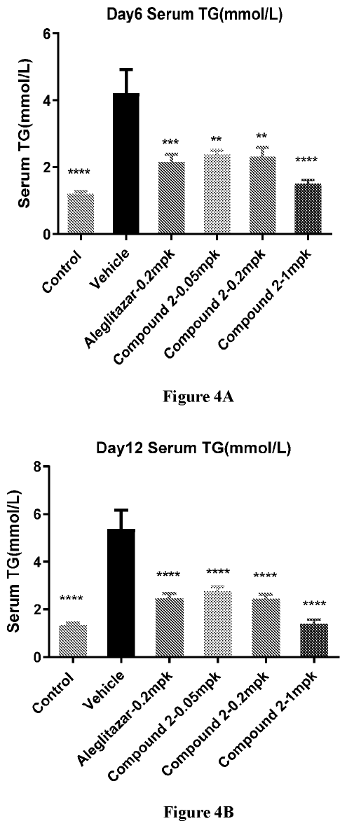

The animals' blood biochemical indicator TG was measured on Day 6 and Day 12, and the results were shown in FIGS. 4A and 4B. As shown in FIGS. 4A and 4B, the serum TG levels of groups treated with either Aleglitazar or Compound 2 at different dosages were significantly lower than those of the vehicle group on Day 6 and Day 12, where the largest effect was observed at Group 6 (Compound 2, 1 mg/kg).

### 16.4.3 Random Blood Glucose

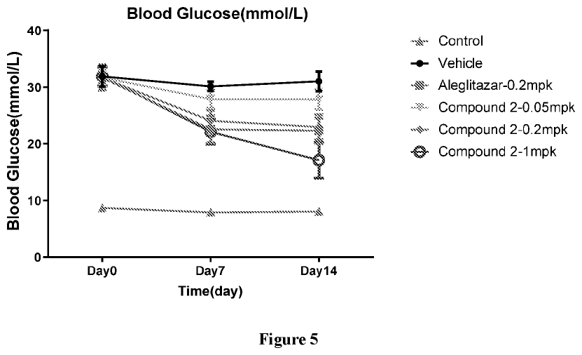

The effects of Aleglitazar and different dosages of Compound 2 on the random blood glucose of db/db model animals during the experimental period were shown in FIG. 5. Compared with the vehicle group, the random blood glucose levels of animals treated with either Aleglitazar or Compound 2 with different dosages were reduced on Day 7. Such reduction reached statistical significance at Group 3 (Aleglitazar, 0.2 mg/kg) and Group 6 (Compound 2, 1 mg/kg) but not in Groups 4 and 5 (Compound 2, 0.05 mg/kg & 0.2 mg/kg). On Day 14, more pronounced effect of blood glucose lowering was observed in Group 6 (Compound 2, 1 mg/kg) vs Group 2 (vehicle) when compared with Day 7, while Groups 3 and 5 (Aleglitazar, 0.2 mg/kg, and Compound 2, 0.2 mg/kg) showed similar effect. On the other hand, such effect in Group 3 (Compound 2, 0.05 mg/kg) still did not reach statistical significance. Thus Compound 2 had a slightly weaker effect on lowering blood glucose when compared with Aleglitazar (Table 7).

TABLE 7
The statistical difference of the blood glucose level
between each treatment group and the vehicle group

| | Blood Glucose(mmol/L) | | |
|---|---|---|---|
| Group | Day 0 | Day 7 | Day 14 |
| Aleglitazar (0.2 mpk) | p > 0.05 | p < 0.05 | p < 0.01 |
| Compound 2 (0.05 mpk) | p > 0.05 | p > 0.05 | p > 0.05 |
| Compound 2 (0.2 mpk) | p > 0.05 | p > 0.05 | p < 0.05 |
| Compound 2 (1 mpk) | p > 0.05 | p < 0.05 | p < 0.0001 |

### 16.4.4 Glucose Tolerance Test in Animals

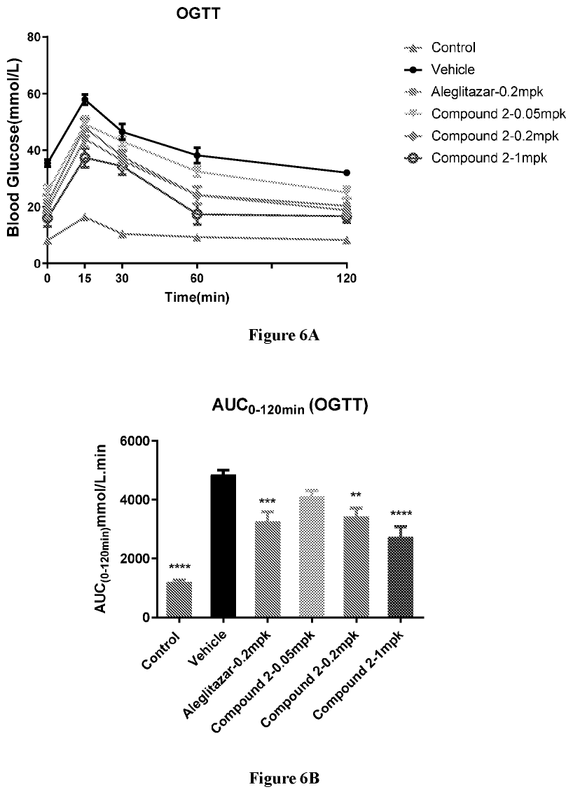

At the end of the experiment, oral glucose tolerance tests were carried out on the db/db animals treated by different compounds. Within 120 minutes after test, the blood glucose value at each time point and the area under the blood glucose-time curve were shown in FIGS. 6A and 6B. Compared with the vehicle group, the blood glucose levels of both Aleglitazar and Compound 2 at different dosages were significantly reduced at each time point. Among them, the AUC₀₋₁₂₀ₘᵢₙ of the test product Aleglitazar (0.2 mg/kg, P<0.001) and Compound 2 (0.2 mg/kg, P<0.01 &1 mg/kg, P<0.0001) was significantly lower than that of the vehicle group. Besides, when compared with Aleglitazar, the AUC₀₋₁₂₀ₘᵢₙ of Compound 2 at the same dose level (0.2 mg/kg) was higher, demonstrating a lower PPARγ activity. Furthermore, the results of Group 4 (Compound 2, 0.05 mg/kg) showed a trend of decrease but did not reach statistical significance at all time points when compared with the vehicle group.

TABLE 8
The statistical difference of the blood glucose level
between each OGTT treatment group
and the vehicle group at each time point.

| | on-treatment period | | | | |
|---|---|---|---|---|---|
| Group | 0 min | 15 min | 30 min | 60 min | 120 min |
| Aleglitazar | p < 0.001 | p < 0.01 | p > 0.05 | p < 0.01 | p < 0.01 |
| (0.2 mpk) | | | | | |
| Compound 2 | p > 0.05 | p > 0.05 | p > 0.05 | p > 0.05 | p > 0.05 |
| (0.05 mpk) | | | | | |
| Compound 2 | p < 0.01 | p > 0.05 | p > 0.05 | p < 0.01 | p < 0.05 |
| (0.2 mpk) | | | | | |
| Compound 2 | p < 0.0001 | p < 0.0001 | p < 0.05 | p < 0.0001 | p < 0.001 |
| (1 mpk) | | | | | |

Note:
Statistical analysis of OGTT vs vehicle (Two-way ANOVA followed by Dunnett test by Prism Graphpad)

### 16.5 Discussion

In the disclosure, we obtained a novel compound with better α/γ activity, i.e., Compound 2.

In vitro transcription activity experiment showed that the EC₅₀ of the compound to activate PPARα and PPARγ pathways was at nanomolar level, indicating that Compound 2 had good in vitro biological activity. Compared with Aleglitazar, Compound 2 showed superior PPARα agonistic activity and weaker PPARγ agonistic ability.

Hyperlipidemia rat model experiment showed that Compound 2 and Compound 4 could effectively and significantly reduce blood lipid levels in animals. In addition, Compound 2 and Compound 4 at a low dose level had a better blood lipid-lowering effect than Aleglitazar at the corresponding concentration. Therefore, the results indicated that Compound 2 and Compound 4 had better PPARα activity at a low dose level, leading to a better lipid-lowering effect.

ICR mouse bodyweight experiment showed that after treatment by low dose level of Compound 2, animals' body weight was comparable to that of the control group with no significant change. In contrast, the same dose level of Aleglitazar caused a significant increase in body weight. As weight increase is a well-known side effect of PPARγ, this demonstrated that, at a low dose level, the PPARγ activity of Compound 2 was weaker than that of Aleglitazar.

The study with db/db mice showed that Compound 2 could effectively reduce the blood glucose level and triglyceride content in type II diabetic mice. It indicated that Compound 2 can exhibit the in vivo biological effect of PPARγ to control blood glucose. Furthermore, Compound 2 at the same dose level can achieve a blood glucose-lowering effect similar to that of Aleglitazar. Therefore, the agonistic effect of Compound 2 on the PPARγ pathway is sufficient to achieve the regulation of glucose homeostasis.

### Example 17: Diabetic Nephropathy Pharmacodynamic Model

### 17.1 Experimental Methods

17.1.1 Animals: five-week-old wild type mice and db/db:BLKS male mice were purchased from Jiangsu GemPharmatech, Co., Ltd. The animals were housed in an SPF environment with 12-hour light/dark cycle. Housing temperature was maintained at 22-26° C. and humidity at 40%-60%. Mice were allowed to access food and water ad libitum. At the age of 6 weeks, db/db mice were anesthetized with 2.5% isopentane and subjected to uninephrectomy with removal of right kidney. Buprenorphine was applied after surgery.

17.1.2 Procedure. Two weeks after surgery, db/db mice were randomly assigned into 5 groups. Wild type mice were used for the control animals. Then total 5 animal groups were included in this study: Group1, control group with 6 animals dosed with vehicle; Group 2, vehicle groups with 10 animals dosed with vehicle; Group 3, Compound-low group with 10 animals dosed with Compound 2 at 0.1 mg/kg; Group 4, Compound-middle group with 10 animals orally dosed with Compound 2 at 0.3 mg/kg; and Group 5, Compound-high group with 10 animals dosed with Compound 2 at 1 mg/kg. Compounds were orally administrated once per day for 10 weeks.

17.1.3 Formulation. The formulation was prepared twice a week. 1. Vehicle: 0.5% sodium carboxymethyl cellulose. 2.5 g sodium carboxymethyl cellulose was weighed and mixed with 500 ml ddH₂O until dissolved completely. 2. Working solution for 1 mg/kg administration: 0.2 mg/ml working solution. 6 mg of compound was added into 30 ml of 0.5% sodium carboxymethyl cellulose, and then vortexed until well suspended. 3. Working solution for 0.3 mg/kg administration: 0.06 mg/ml working solution. 6 ml of 0.1 mg/ml compound solution was mixed with 14 ml of 0.5% sodium carboxymethyl cellulose, and then stirred until well suspended. 4. Working solution for 0.1 mg/kg administration: 0.02 mg/ml working solution. 2 ml of 0.2 mg/ml compound solution was mixed with 18 ml of 0.5% sodium carboxymethyl cellulose, and then stirred until well suspended.

At the 5^{th} and 9^{th} week after compound administration, mice were placed in metabolic cages for urine collection. Albumin level was measured for 24 hr albumin excretion calculation. At the 10^{th} week after treatment, the animals were sacrificed for kidney dissection. The kidney was fixed in 10% neutral buffed formalin, then paraffin-embedded for histopathological analysis. Glomerulosclerosis was assessed by evaluating glomerular basement membrane, mesangial expansion, nodular sclerosis and glomerulosclerosis. Severity was graded as follows: 0: normal; 1: Glomerular basement membrane thickening: isolated glomerular basement membrane thickening and only mild, nonspecific changes by light microscopy: 2: Mesangial expansion mild (IIa) or severe (IIb): glomeruli with mild or severe mesangial expansion but without nodular sclerosis or global glomerulosclerosis in more than 50% of glomeruli; 3: Nodular sclerosis: at least one glomerulus with nodular increase in mesangial matrix; 4: Advanced diabetic glomerulosclerosis: more than 50% global glomerulosclerosis with other clinical or pathological evidence that sclerosis is attributable to diabetic nephropathy. Renal tubular injury was scored as follows: 0: no obvious lesions; 1: up to 25% involvement of tubular lesion; 2: lesion in 25% to 50% of renal tubular; 3: 50% to 75% of renal tubular lesion and grade 4: >75% tubular lesion.

17.1.4 Data were presented as mean±SEM. Graphpad 8.0 software was used for statistical analysis. The difference between the values were analyzed with one-way ANOVA. The difference between histopathological score was analyzed with Kruskal-Wallis nonparameter test. All values were compared with the group 2.

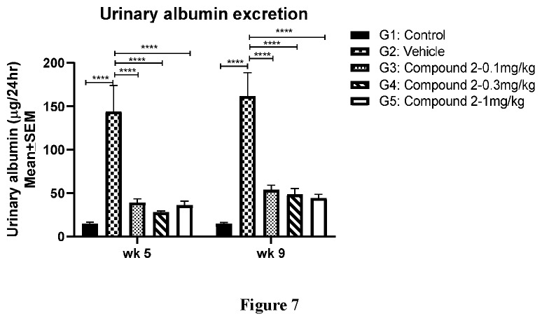

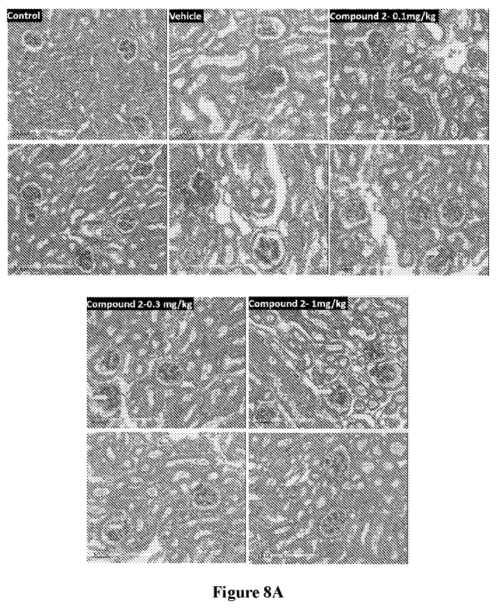

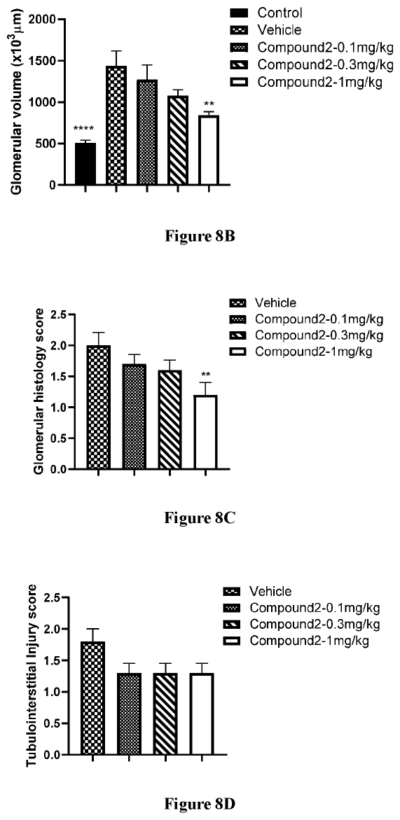

17.2 Results. 1. 24-hour urinary albumin. In the vehicle-treated uninephrectomied db/db mice, 24-hour urinary albumin increased by more than 10 folds compared with the control. Compound 2 significantly decreased 24-hour urinary albumin at the 5^{th} and 9^{th} weeks after compound administration. More than 50% reduction of urine albumin was achieved with Compound 2 treatment (FIG. 7). 2. Glomerulosclerosis. As shown in FIG. 8A, control animals had normal glomeruli appearance and glomerular volume. However, mesangial expansion, glomerular basement membrane thickening, and nodular sclerosis were observed in the vehicle-treated db/db animals. Compound 2 suppressed the glomerular damage and ameliorate the glomerular hypertrophy. Both histopathological score and glomerular volume were statistically significantly reduced after animal receiving high dose of Compound 2 (FIGS. 8B and 8C). 3. Renal tubular injury. Histopathological analysis showed control animals had normal tubular structure, but vehicle treated animals developed tubular dilation, basement membrane thickening/tubular atrophy and tubular cast. Compound 2 at all dosage levels improved the tubular injury at some extend (FIG. 8D).

### Example 18: Effect of Compound 2 on Improvement of Renal Injury in Rat Model of Unilateral Ureteral Obstruction

### 18.1 Experimental Methods:

18.1.1 Male SD rats weighing 240-260 g were housed in an SPF environment with 12-hour light/dark cycle. Housing temperature was maintained at 20-26° C. and humidity at 40%-60%. Rats were fed with a standard chow and allowed to access food and water ad libitum.

18.1.2 Formulation. The formulation was prepared twice a week. 1. Vehicle: 0.5% sodium carboxymethyl cellulose was prepared as described in 17.1.3. 2. 0.2 mg/ml solution of either Compound 2 or Aleglitazar. 6 mg of compound was added into 30 ml of 0.5% sodium carboxymethyl cellulose, and then vortexed until well suspended. 3. 0.02 mg/ml solution of either Compound 2 or Aleglitazar for the dosage of 0.2 mg/kg. 2 ml of 0.2 mg/ml solution was mixed with 18 ml of 0.5% sodium carboxymethyl cellulose, and then stirred until well suspended.

18.1.3 Procedure. After acclimation, animals are randomly assigned into the following groups: control group with 8 rats dosed with vehicle at 10 ml/kg; model group with 10 rats dosed with vehicle at 10 ml/kg, reference group with 10 animals dosed with Aleglitazar at 0.2 mg/kg; and compound group with 10 animals dosed with Compound 2 at 0.2 mg/kg. Vehicle or compounds were administrated by orally gavage daily. Unilateral ureteral obstruction was performed on the second day of compound treatment. Animals in model group, reference group and compound group underwent urethral ligation under isoflurane anesthesia one hour after receiving compound administration. A flank incision was cut to expose the left ureter, and a 4-0 surgical suture was used to perform two-point ligation to achieve urethral obstruction. Ureter was cut between the two ligation points. Animals in the control group were subjected to the same surgical treatment except the ligation and cut. After surgery, animals in each group continually received vehicle or compound for 12 more days, for a total of 14 treatment days.

Rats were sacrificed after 14 days' compound treatment. The obstructed kidney was weighed and collected for histology. The tissue samples were fixed with formalin, and then paraffin embedded. The embedded tissue was sectioned and stained with hematoxylin & eosin and Masson trichrome to assess kidney structure and fibrosis. Focal lesions included infract, tubular dilation, tubular obstruction and necrosis which were given score respectively between 0 and 4 as the percentage of the affected area of the biopsy (score 0: No, 1: <25%; 2: 25%-50%, 3: 50%-75%, 4: >75%). The severity of renal injury was evaluated by the total scores of the lesions. Kidney fibrosis was graded as 0-4 based on the percentage of area stained with Masson trichrome (score 0: No, 1: <25%; 2: 25%-50%, 3: 50%-75%, 4: >75%).

18.1.4 Data were presented as Mean±SEM. The multiple comparison was analyzed with the Kruskal-Wallis non-parameter test. The Dunn's test was used to compare the difference to the model group. A p value <0.05 was considered statistically significant.

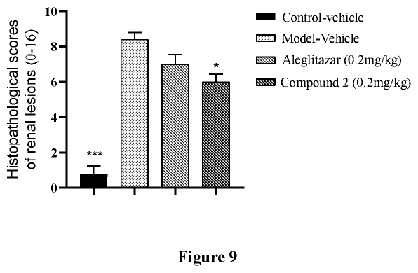

18.1.5 Results. When compared to the model group dosed with vehicle, Compound 2 at 0.2 mg/kg significantly improved the total score comprised of renal focal lesions, covering infract, tubular dilation, tubular obstruction, fibrosis, and necrosis (FIG. 9). Aleglitazar, the reference compound, on the other hand, did not achieve statistically significant improvement in total score at the same dose of 0.2 mg/kg. Therefore, compound 2 is superior to Aleglitazar in preventing and alleviating the renal injury caused by unilateral ureteral obstruction.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A compound of formula (I), or its pharmaceutical acceptable salt, 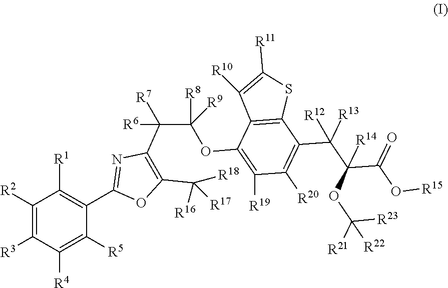
wherein, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ independently from each other are H or D, and at least one, two, three, four, five, six, seven, eight, nine, ten, eleven, twelve, thirteen, fourteen, fifteen, sixteen, seventeen, eighteen, nineteen, twenty, twenty one, twenty two or twenty three of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ is D.

2. The compound or its pharmaceutical acceptable salt of claim 1, wherein no more than one, two, three, four, five, six, seven, eight, nine, ten, eleven, twelve, thirteen, fourteen, fifteen, sixteen, seventeen, eighteen, nineteen, twenty, twenty one, twenty two or twenty three of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ is D.

3. The compound or its pharmaceutical acceptable salt of claim 1, wherein at least one, two, three or four of R⁶, R⁷, R⁸ and R⁹ is D.

4. The compound or its pharmaceutical acceptable salt of claim 1, wherein no more than one, two, three or four of R⁶, R⁷, R⁸ and R⁹ is D.

5. The compound or its pharmaceutical acceptable salt of claim 1, wherein one or two of R⁶ or R⁷ is D.

6. The compound or its pharmaceutical acceptable salt of claim 1, wherein one or two of R⁸ or R⁹ is D.

7. The compound or its pharmaceutical acceptable salt of claim 3 or 4, wherein all of R¹, R², R³, R⁴, R⁵, R¹⁰, R¹¹, R¹², R¹³, R¹⁴, R¹⁵, R¹⁶, R¹⁷, R¹⁸, R¹⁹, R²⁰, R²¹, R²² and R²³ are H.

8. The compound or its pharmaceutical acceptable salt of claim 5, wherein all of R¹, R², R³, R⁴, R⁵, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ are H.

9. The compound or its pharmaceutical acceptable salt of claim 6, wherein all of R¹, R², R³, R⁴, R⁵, R⁶, R⁷, R¹⁰, R¹¹, R¹², R¹³, R¹⁴, R¹⁵, R¹⁶, R¹⁷, R¹⁸, R¹⁹, R²⁰, R²¹, R²² and R²³ are H.

10. The compound or its pharmaceutical acceptable salt of claim 1, wherein at least one, two, three, four or five of R¹, R², R³, R⁴ and R⁵ is D.

11. The compound or its pharmaceutical acceptable salt of claim 1, wherein no more than one, two, three, four or five of R¹, R², R³, R⁴ and R⁵ is D.

12. The compound or its pharmaceutical acceptable salt of claim 10 or 11, wherein all of R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ are H.

13. The compound or its pharmaceutical acceptable salt of claim 1, wherein all of R¹, R², R³, R⁴ and R⁵ are D, and all of R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ are H.

14. The compound or its pharmaceutical acceptable salt of claim 1, wherein at least one, two, three, four, five, six, seven, eight or nine of R¹, R², R³, R⁴, R⁵, R⁶, R⁷, R⁸ and R⁹ is D.

15. The compound or its pharmaceutical acceptable salt of claim 1, wherein no more than one, two, three, four, five, six, seven, eight or nine of R¹, R², R³, R⁴, R⁵, R⁶, R⁷, R⁸ and R⁹ is D.

16. The compound or its pharmaceutical acceptable salt of claim 1, wherein at least one, two, three, four or five of R¹, R², R³, R⁴ and R$ is D and at least one, two, three, or four of R⁶, R⁷, R⁸ and R⁹ is D.

17. The compound or its pharmaceutical acceptable salt of claim 1, wherein at least one, two, three, four or five of R¹, R², R³, R⁴ and R⁵ is D and at least one or two of R⁸ and R⁹ is D.

18. The compound or its pharmaceutical acceptable salt of claim 1, wherein at least one or two of R¹ and R⁵ is D and at least one or two of R⁸ and R⁹ is D.

19. The compound or its pharmaceutical acceptable salt of claim 14 or 15, wherein all of R¹⁰, R¹¹, R¹², R¹³, R¹⁴, R¹⁵, R¹⁶, R¹⁷, R¹⁸, R¹⁹, R²⁰, R²¹, R²² and R²³ are H.

20. The compound or its pharmaceutical acceptable salt of claim 1, wherein at least one, two or three of R¹², R¹³ and R¹⁴ is D.

21. The compound or its pharmaceutical acceptable salt of claim 1, wherein no more than one, two or three of R¹², R¹³ and R¹⁴ is D.

22. The compound or its pharmaceutical acceptable salt of claim 1, wherein at least one or two of R¹² and R¹³ is D and R¹⁴ is D.

23. The compound or its pharmaceutical acceptable salt of claim 20 or 21, wherein all of R¹, R², R³, R⁴, R⁵, R⁶, R⁷, R⁸, R⁹, R¹⁰, R¹¹, R¹⁵, R¹⁶, R¹⁷, R¹⁸, R¹⁹, R²⁰, R²¹, R²² and R²³ are H.

24. The compound or its pharmaceutical acceptable salt of claim 1, wherein at least one, two or three of R¹⁶, R¹⁷ and R¹⁸ is D.

25. The compound or its pharmaceutical acceptable salt of claim 1, wherein no more than one, two or three of R¹⁶, R¹⁷ and R¹⁸ is D.

26. The compound or its pharmaceutical acceptable salt of claim 24 or 25, wherein all of R¹, R², R³, R⁴, R⁵, R⁶, R⁷, R⁸, R⁹, R¹⁰, R¹¹, R¹², R¹³, R¹⁴, R¹⁵, R¹⁹, R²⁰, R²¹, R²² and R²³ are H.

27. The compound or its pharmaceutical acceptable salt of claim 1, wherein at least one, two, three, four, five, six, seven, eight, nine, ten, eleven or twelve of R¹, R², R³, R⁴, R⁵, R⁶, R⁷, R⁸, R⁹, R¹⁶, R¹⁷ and R¹⁸ is D.

28. The compound or its pharmaceutical acceptable salt of claim 1, wherein no more than one, two, three, four, five, six, seven, eight, nine, ten, eleven or twelve of R¹, R², R³, R⁴, R⁵, R⁶, R⁷, R⁸, R⁹, R¹⁶, R¹⁷ and R¹⁸ is D.

29. The compound or its pharmaceutical acceptable salt of claim 1, wherein at least one or two of R⁸ and R′ is D and at least one, two or three of R¹⁶, R¹⁷ and R¹⁸ is D.

30. The compound or its pharmaceutical acceptable salt of claim 1, wherein at least one, two, three, four or five of R¹, R², R³, R⁴, and R⁵ is D and at least one, two or three of R¹⁶, R¹⁷ and R¹⁸ is D.

31. The compound or its pharmaceutical acceptable salt of claim 27 or 28, wherein all of, R¹⁰, R¹¹, R¹², R¹³, R¹⁴, R¹⁵, R¹⁹, R²⁰, R²¹, R²² and R²³ are H.

32. A compound of formula (Ia), or its pharmaceutical acceptable salt, 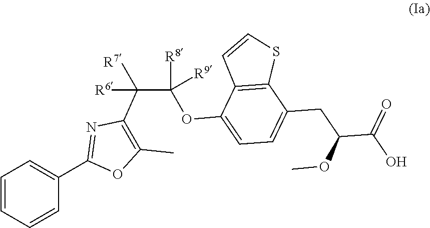
wherein, R^{6′}, R^{7′}, R^{8′} and R^{9′} independently from each other are H or D, wherein at least one, two, three or four of R^{6′}, R^{7′}, R^{8′} and R^{9′} is D.

33. The compound or its pharmaceutical acceptable salt of claim 32, wherein no more than one, two, three or four of R^{6′}, R^{7′}, R^{8′} and R^{9′} is D.

34. The compound or its pharmaceutical acceptable salt of claim 32, wherein at least one or two of R^{8′} and R^{9′} is D.

35. The compound or its pharmaceutical acceptable salt of claim 32, wherein at least one or two of R⁶ and R^{7′} is D.

36. The compound or its pharmaceutical acceptable salt of claim 32, wherein both of R^{6′} and R^{7′} are H when at least one or two of R^{8′} and R^{9′} is D.

37. The compound or its pharmaceutical acceptable salt of claim 32, wherein both of R^{6′} and R^{7′} are H when both of R^{8′} and R^{9′} is D.

38. The compound or its pharmaceutical acceptable salt of claim 32, wherein both of R^{8′} and R^{9′} are H when at least one or two R^{6′} and R^{7′} of is D.

39. A compound selected from 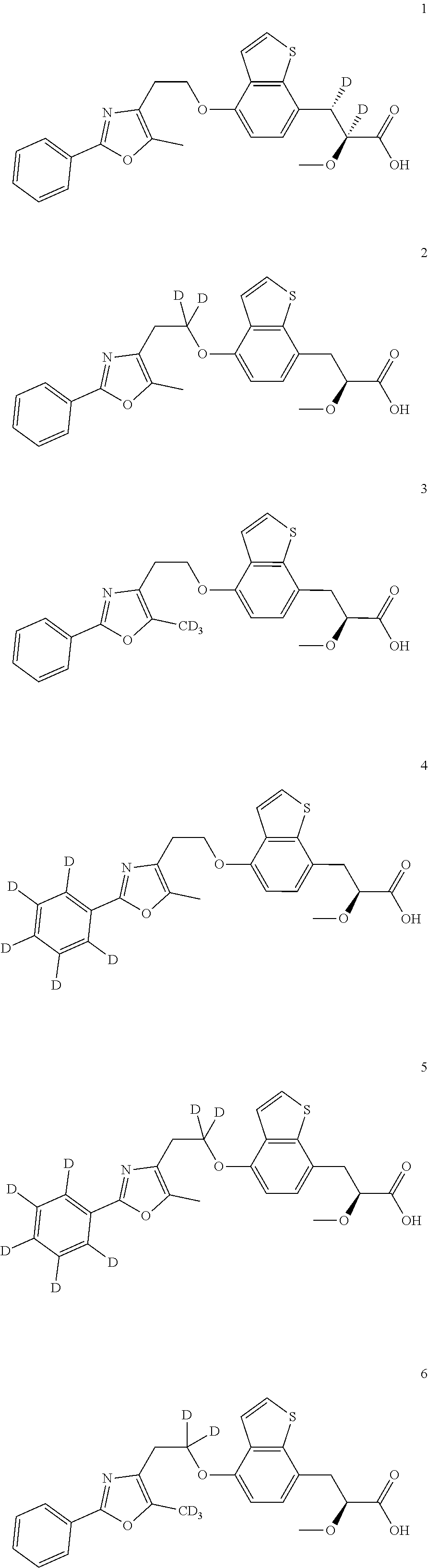 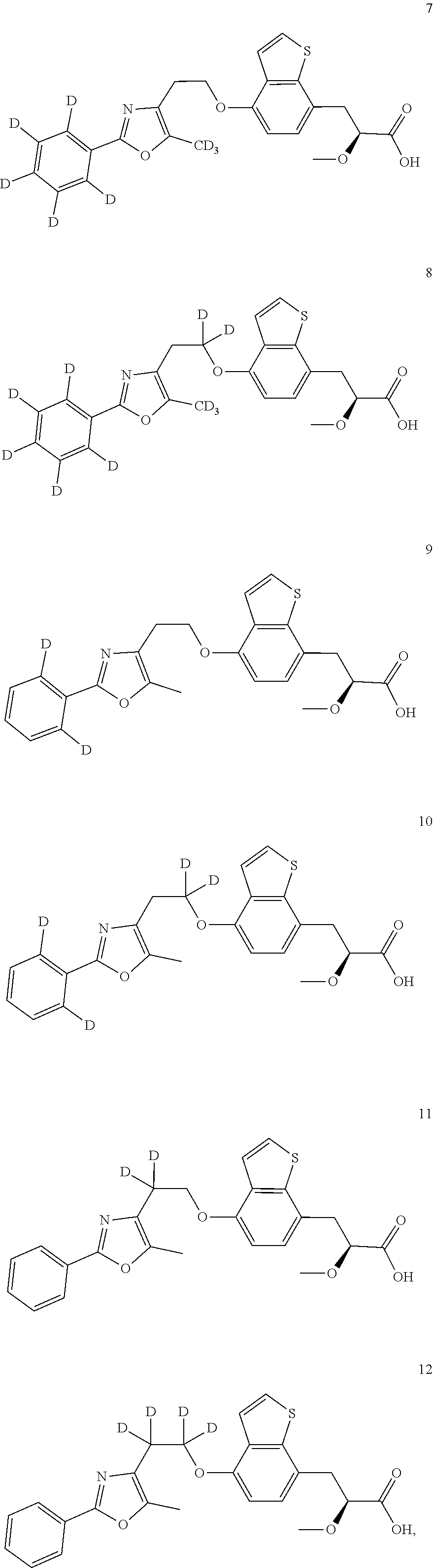
or its pharmaceutical acceptable salt.

40. The compound or its pharmaceutical acceptable salt of any one of claims 1-39, wherein deuterium enrichment is no less than 50%, 60%, 70%, 80%, 90%, 95%, 96%, 97%, 98% or 99%.

41. The compound or its pharmaceutical acceptable salt of any one of claims 1-40, wherein deuterium enrichment is no more than 99.9%, 99%, 98%, 97%, 96%, 95%, or 90%.

42. A pharmaceutical composition comprising a compound or its pharmaceutical acceptable salt of any one of claims 1-41 and a pharmaceutically acceptable carrier and/or adjuvant.

43. A dual agonist of PPARα and PPARγ for use in a method for the treatment and/or prophylaxis of diseases which are modulated by PPARα and/or PPARγ agonists, wherein the dual agonist of PPARα and PPARγ is deuterated.

44. A method for the treatment and/or prophylaxis of diseases which are modulated by PPARα and/or PPARγ agonists in a subject, comprising administering a dual agonist of PPARα and PPARγ to the subject, wherein the dual agonist of PPARα and PPARγ is deuterated.

45. The use of a dual agonist of PPARα and PPARγ in the manufacture of a medicament for the treatment and/or prophylaxis of diseases which are modulated by PPARα and/or PPARγ agonists, wherein the dual agonist of PPARα and PPARγ is deuterated.

46. The use and/or the method of any one of claims 43-45, wherein the disease is diabetes, non-insulin dependent diabetes mellitus, elevated blood pressure, dyslipidemia, atherosclerotic diseases, metabolic syndrome, or diabetic nephropathy.

47. The use and/or the method of any one of claims 43-45, wherein the disease is renal injury.

48. The use and/or the method of claim 47, wherein the renal injury is caused by ureteral obstruction.

49. The use and/or the method of claim 47, wherein the renal injury is caused by unilateral ureteral obstruction.

50. The use and/or the method of any one of claims 43-45, wherein the dual agonist of PPARα and PPARγ is deuterated Aleglitazar or its pharmaceutical acceptable salt.

51. The use and/or the method of any one of claims 43-45, wherein the dual agonist of PPARα and PPARγ is the compound or its pharmaceutical acceptable salt of any one of claims 1-41.

52. A method for modulating the specific agonistic activity of PPARα or PPARγ of a dual agonist of PPARα and PPARγ, comprising deuterating the agonist.

53. A method for improving the specific agonistic activity of PPARα or PPARγ of a dual agonist of PPARα and PPARγ, comprising deuterating the agonist.

54. The method of claim 52 or 53, wherein the specific agonistic activity of PPARα of the agonist is improved.

55. The method of claim 52 or 53, wherein the specific agonistic activity of PPARγ of the agonist is improved.

56. The method of claim 52 or 53, wherein at least one, two, three, four, five, six, seven, eight, nine, ten of H of the agonist is deuterated.

57. The method of claim 52 or 53, wherein no more than one, two, three, four, five, six, seven, eight, nine, ten of H of the agonist is deuterated.

58. The method of claim 52 or 53, wherein the dual agonist of PPARα and PPARγ is Aleglitazar or its pharmaceutical acceptable salt.
